(19) United States
(12) Patent Application Publication
(10) Pub. No.: US 2023/0133538 A1
(43) Pub. Date: May 4, 2023

(54) TARGETED DEGRADERS OF ABERRANT TAU BASED ON THE PET TRACER PBB3

(71) Applicants: DANA-FARBER CANCER INSTITUTE, INC., Boston, MA (US); THE GENERAL HOSPITAL CORPORATION, Boston, MA (US)

(72) Inventors: Fleur M. Ferguson, Cambridge, MA (US); Nathanael S. Gray, Stanford, CA (US); Stephen J. Haggarty, Gloucester, MA (US); Maria Catarina Telo Baptista Lima Da Silva, Revere, MA (US)

(73) Assignees: DANA-FARBER CANCER INSTITUTE, INC., Boston, MA (US); THE GENERAL HOSPITAL CORPORATION, Boston, MA (US)

(21) Appl. No.: 17/912,272

(22) PCT Filed: Mar. 17, 2021
(86) PCT No.: PCT/US21/22758

§ 371 Date: Sep. 16, 2022

Related U.S. Application Data
(60) Provisional application No. 62/991,359, filed on Mar. 18, 2020.

(51) Int. Cl. C07D 417/06; C07D 413/06; C07D 401/04; C07D 403/04; C07D 417/14; C07D 487/04

(52) U.S. Cl. CPC C07D 417/06; C07D 413/06; C07D 401/04; C07D 403/04; C07D 417/14; C07D 487/04

(57) ABSTRACT

Disclosed are bispecific compounds (degraders) that target tau protein for degradation. Also disclosed are pharmaceutical compositions containing the degraders and methods of using the compounds to treat neurodegenerative and neuropsychiatric diseases associated with aberrant tau.

### RELATED APPLICATIONS

This application claims the benefit of priority under 35 U.S.C. § 119(e) to U.S. Provisional Application No. 62/991,359, filed Mar. 18, 2020, which is incorporated herein by reference in its entirety.

### BACKGROUND OF THE INVENTION

Tau protein is a microtubule-associated protein (MAP) that is abundantly expressed in neurons of the central nervous system (Okamura et al., Clin. Transl. Imaging 6:305-316 (2018)). Tau proteins interact with tubulin to stabilize microtubules and promote tubulin assembly into microtubules. The stabilization of microtubules is controlled through isoforms or phosphorylation (Cleveland et al., J. Mol. Biol. 116:207-225 (1977)). Tauopathies are a group of neurodegenerative diseases that are pathologically defined by the presence of tau protein aggregates in the brain (Orr et al., Trends Pharmacol. Sci. 38:637-648 (2017)). Tau protein aggregates are composed of tau proteins that have become defective and no longer stabilize microtubules properly, typically associated with hyper-phosphorylation. Tau has been implicated in the pathogenesis of autism and related neurodevelopment disorders where the reduction of tau is a potential therapeutic strategy for treating these disorders (Tai et al., Neuron, pii:S0896-6273(20):30065-9 (2020)). Currently, there are no FDA-approved small molecules or other therapeutic modalities that selectively target tau for the treatment of neurodegenerative, neuropsychiatric, neurological and other disorders. Accordingly, improved techniques are needed in order to overcome the challenges of combating aberrant tau related diseases.

### SUMMARY OF THE INVENTION

A first aspect of the present invention is directed to a bispecific compound of formula (I),

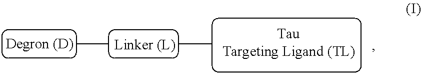

wherein the targeting ligand represents a moiety that binds tau, the degron represents a moiety that binds an E3 ubiquitin ligase or the degron is an autophagy-recruiting tag (i.e. a tag that destines or targets substrates for selective autophagy), and the linker represents a moiety that covalently connects the degron and the targeting ligand, or a pharmaceutically acceptable salt or stereoisomer thereof,

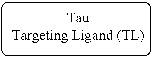

wherein represents formula TL-1 or TL-2:

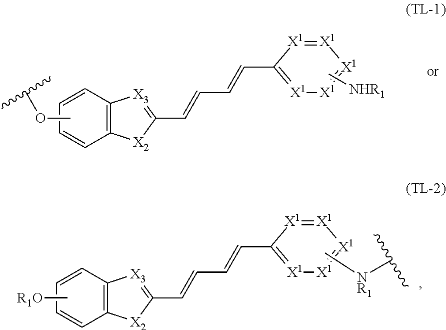

wherein each X₁, X₂, X₃, and R₁ are defined herein, or a pharmaceutically acceptable salt or stereoisomer thereof.

Another aspect of the present invention is directed to a pharmaceutical composition containing a therapeutically effective amount of the bispecific compound of formula (I) or a pharmaceutically acceptable salt or stereoisomer thereof, and a pharmaceutically acceptable carrier.

In another aspect of the present invention, methods of making the bispecific compounds are provided.

A further aspect of the present invention is directed to a method of treating a disease or disorder that is characterized or mediated by aberrant tau protein activity, comprising administering to a subject in need thereof a therapeutically effective amount of the bispecific compound of formula (I) or a pharmaceutically acceptable salt or stereoisomer thereof.

Without intending to be bound by any particular theory of operation, the bispecific compounds of formula (I) (also referred to herein as PROTACs or degraders) are believed to promote the degradation of tau protein via cells' Ubiquitin/Proteasome System, whose function is to routinely identify and remove damaged proteins. After destruction of a tau molecule, the degrader is released and continues to be active. Thus, by engaging and exploiting the body's own natural protein disposal system, the bispecific compounds of the present invention may represent a potential improvement over current small molecule inhibitors of tau protein. Thus, effective intracellular concentrations of the degraders may be significantly lower than for small molecule tau inhibitors. Bispecific compounds of the present invention may be more potent inhibitors of tau protein than known inhibitors.

Accordingly, the bispecific compounds of the present invention may offer at least one additional advantage including improved pharmacodynamics. The use of targeted degradation technology to recruit E3-ligase adaptor proteins to tau protein aggregates via the bispecific compound leads to ubiquitination and clearance through the proteasome. Collectively, the present bispecific compounds may represent an advancement in the field.

### BRIEF DESCRIPTION OF THE DRAWINGS

FIG. 1 is an immunoblot showing the degradation of P-Tau S396 after 24 hour treatment of inventive bispecific compounds 2 and 4 and a negative control in Tau-A152T neurons at indicated concentrations (μM).

FIG. 2 is a series of graphs for bispecific compounds 2 and 4 and a negative control in depicting dose-effect in Tau-A152T neurons.

FIG. 3 is an immunoblot showing the degradation of P-Tau S396 after 24 hour treatment of bispecific compounds 2 and 4 and a negative control in Tau-P301L neurons at indicated concentrations (μM).

FIG. 4 is a series of graphs for bispecific compounds 2 and 4 and a negative control in depicting dose-effect in Tau-P301L neurons.

### DETAILED DESCRIPTION OF THE INVENTION

Unless defined otherwise, all technical and scientific terms used herein have the same meaning as is commonly understood by one of skill in the art to which the subject matter herein belongs. As used in the specification and the appended claims, unless specified to the contrary, the following terms have the meaning indicated in order to facilitate the understanding of the present invention.

As used in the description and the appended claims, the singular forms “a”, “an”, and “the” include plural referents unless the context clearly dictates otherwise. Thus, for example, reference to “a composition” includes mixtures of two or more such compositions, reference to “an inhibitor” includes mixtures of two or more such inhibitors, and the like.

Unless stated otherwise, the term “about” means within 10% (e.g., within 5%, 2% or 1%) of the particular value modified by the term “about.”

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

With respect to compounds of the present invention, and to the extent the following terms are used herein to further describe them, the following definitions apply.

As used herein, the term “alkyl” refers to a saturated linear or branched-chain monovalent hydrocarbon radical. In one embodiment, the alkyl radical is a C₁-C₁₈ group. In other embodiments, the alkyl radical is a C₀-C₆, C₀-C₅, C₀-C₃, C₁-C₁₂, C₁-C₈, C₁-C₆, C₁-C₅, C₁-C₄ or C₁-C₃ group (wherein C₀ alkyl refers to a bond). Examples of alkyl groups include methyl, ethyl, 1-propyl, 2-propyl, i-propyl, 1-butyl, 2-methyl-1-propyl, 2-butyl, 2-methyl-2-propyl, 1-pentyl, n-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, heptyl, octyl, nonyl, decyl, undecyl and dodecyl. In some embodiments, an alkyl group is a C₁-C₃ alkyl group. In some embodiments, an alkyl group is a C₁-C₂ alkyl group, or a methyl group.

As used herein, the term “alkylene” refers to a straight or branched divalent hydrocarbon chain linking the rest of the molecule to a radical group, consisting solely of carbon and hydrogen, containing no unsaturation and having from one to 12 carbon atoms, for example, methylene, ethylene, propylene, n-butylene, and the like. The alkylene chain may be attached to the rest of the molecule through a single bond and to the radical group through a single bond. In some embodiments, the alkylene group contains one to 8 carbon atoms (C₁-C₈ alkylene). In other embodiments, an alkylene group contains one to 5 carbon atoms (C₁-C₅ alkylene). In other embodiments, an alkylene group contains one to 4 carbon atoms (C₁-C₄ alkylene). In other embodiments, an alkylene contains one to three carbon atoms (C₁-C₃ alkylene). In other embodiments, an alkylene group contains one to two carbon atoms (C₁-C₂ alkylene). In other embodiments, an alkylene group contains one carbon atom (C₁ alkylene).

As used herein, the term “alkenyl” refers to a linear or branched-chain monovalent hydrocarbon radical with at least one carbon-carbon double bond. An alkenyl includes radicals having “cis” and “trans” orientations, or alternatively, “E” and “Z” orientations. In one example, the alkenyl radical is a C₂-C₁₈ group. In other embodiments, the alkenyl radical is a C₂-C₁₂, C₂-C₂-C₈, C₂-C₆ or C₂-C₃ group. Examples include ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-diene, hex-1-enyl, hex-2-enyl, hex-3-enyl, hex-4-enyl and hexa-1,3-dienyl.

As used herein, the term “alkynyl” refers to a linear or branched monovalent hydrocarbon radical with at least one carbon-carbon triple bond. In one example, the alkynyl radical is a C₂-C₁₈ group. In other examples, the alkynyl radical is C₂-C₁₂, C₂-C₁₀, C₂-C₈, C₂-C₆ or C₂-C₃. Examples include ethynyl prop-1-ynyl, prop-2-ynyl, but-1-ynyl, but-2-ynyl and but-3-ynyl.

The terms “alkoxyl” or “alkoxy” as used herein refer to an alkyl group, as defined above, having an oxygen radical attached thereto, and which is the point of attachment. Representative alkoxyl groups include methoxy, ethoxy, propyloxy, tert-butoxy and the like. An “ether” is two hydrocarbyl groups covalently linked by an oxygen. Accordingly, the substituent of an alkyl that renders that alkyl an ether is or resembles an alkoxyl, such as can be represented by one of —O-alkyl, —O-alkenyl, and —O-alkynyl.

As used herein, the term “halogen” (or “halo” or “halide”) refers to fluorine, chlorine, bromine, or iodine.

As used herein, the term “cyclic group” broadly refers to any group that used alone or as part of a larger moiety, contains a saturated, partially saturated or aromatic ring system e.g., carbocyclic (cycloalkyl, cycloalkenyl), heterocyclic (heterocycloalkyl, heterocycloalkenyl), aryl and heteroaryl groups. Cyclic groups may have one or more (e.g., fused) ring systems. Thus, for example, a cyclic group can contain one or more carbocyclic, heterocyclic, aryl or heteroaryl groups.

As used herein, the term “carbocyclic” (also “carbocyclyl”) refers to a group that used alone or as part of a larger moiety, contains a saturated, partially unsaturated, or aromatic ring system having 3 to 20 carbon atoms, that is alone or part of a larger moiety (e.g., an alkcarbocyclic group). The term carbocyclyl includes mono-, bi-, tri-, fused, bridged, and spiro-ring systems, and combinations thereof. In one embodiment, carbocyclyl includes 3 to 15 carbon atoms (C₃-C₁₅). In one embodiment, carbocyclyl includes 3 to 12 carbon atoms (C₃-C₁₂). In another embodiment, carbocyclyl includes C₃-C₈, C₃-C₁₀ or C₅-C₁₀. In another embodiment, carbocyclyl, as a monocycle, includes C₃-C₈, C₃-C₆ or C₅-C₆. In some embodiments, carbocyclyl, as a bicycle, includes C₇-C₁₂. In another embodiment, carbocyclyl, as a spiro system, includes C₅-C₁₂. Representative examples of monocyclic carbocyclyls include cyclopropyl, cyclobutyl, cyclopentyl, 1-cyclopent-1-enyl, 1-cyclopent-2-enyl, 1-cyclopent-3-enyl, cyclohexyl, perdeuteriocyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, phenyl, and cyclododecyl; bicyclic carbocyclyls having 7 to 12 ring atoms include [4,3], [4,4], [4,5], [5,5], [5,6] or [6,6] ring systems, such as for example bicyclo[2.2.1]heptane, bicyclo[2.2.2]octane, naphthalene, and bicyclo[3.2.2]nonane. Representative examples of spiro carbocyclyls include spiro[2.2]pentane, spiro[2.3]hexane, spiro[2.4]heptane, spiro[2.5]octane and spiro[4.5]decane. The term carbocyclyl includes aryl ring systems as defined herein. The term carbocycyl also includes cycloalkyl rings (e.g., saturated or partially unsaturated mono-, bi-, or spiro-carbocycles). The term carbocyclic group also includes a carbocyclic ring fused to one or more (e.g., 1, 2 or 3) different cyclic groups (e.g., aryl or heterocyclic rings), where the radical or point of attachment is on the carbocyclic ring.

Thus, the term carbocyclic also embraces carbocyclylalkyl groups which as used herein refer to a group of the formula —R^{c}-carbocyclyl where R^{c} is an alkylene chain. The term carbocyclic also embraces carbocyclylalkoxy groups which as used herein refer to a group bonded through an oxygen atom of the formula —O—R^{c}-carbocyclyl where R^{c} is an alkylene chain.

As used herein, the term “aryl” used alone or as part of a larger moiety (e.g., “aralkyl”, wherein the terminal carbon atom on the alkyl group is the point of attachment, e.g., a benzyl group), “aralkoxy” wherein the oxygen atom is the point of attachment, or “aroxyalkyl” wherein the point of attachment is on the aryl group) refers to a group that includes monocyclic, bicyclic or tricyclic, carbon ring system, that includes fused rings, wherein at least one ring in the system is aromatic. In some embodiments, the aralkoxy group is a benzoxy group. The term “aryl” may be used interchangeably with the term “aryl ring”. In one embodiment, aryl includes groups having 6-18 carbon atoms. In another embodiment, aryl includes groups having 6-10 carbon atoms. Examples of aryl groups include phenyl, naphthyl, anthracyl, biphenyl, phenanthrenyl, naphthacenyl, 1,2,3,4-tetrahydronaphthalenyl, 1H-indenyl, 2,3-dihydro-1H-indenyl, naphthyridinyl, and the like, which may be substituted or independently substituted by one or more substituents described herein. A particular aryl is phenyl. In some embodiments, an aryl group includes an aryl ring fused to one or more (e.g., 1, 2 or 3) different cyclic groups (e.g., carbocyclic rings or heterocyclic rings), where the radical or point of attachment is on the aryl ring.

Thus, the term aryl embraces aralkyl groups (e.g., benzyl) which as disclosed above refer to a group of the formula —R^{c}-aryl where R^{c} is an alkylene chain such as methylene or ethylene. In some embodiments, the aralkyl group is an optionally substituted benzyl group. The term aryl also embraces aralkoxy groups which as used herein refer to a group bonded through an oxygen atom of the formula —O—R^{c}-aryl where R^{c} is an alkylene chain such as methylene or ethylene.

As used herein, the term “heterocyclyl” refers to a “carbocyclyl” that used alone or as part of a larger moiety, contains a saturated, partially unsaturated or aromatic ring system, wherein one or more (e.g., 1, 2, 3, or 4) carbon atoms have been replaced with a heteroatom (e.g., O, N, N(O), S, S(O), or S(O)₂). The term heterocyclyl includes mono-, bi-, tri-, fused, bridged, and spiro-ring systems, and combinations thereof. In some embodiments, a heterocyclyl refers to a 3 to 15 membered heterocyclyl ring system. In some embodiments, a heterocyclyl refers to a 3 to 12 membered heterocyclyl ring system. In some embodiments, a heterocyclyl refers to a saturated ring system, such as a 3 to 12 membered saturated heterocyclyl ring system. In some embodiments, a heterocyclyl refers to a heteroaryl ring system, such as a 5 to 14 membered heteroaryl ring system. The term heterocyclyl also includes C₃-C₈ heterocycloalkyl, which is a saturated or partially unsaturated mono-, bi-, or spiro-ring system containing 3-8 carbons and one or more (1, 2, 3 or 4) heteroatoms.

In some embodiments, a heterocyclyl group includes 3-12 ring atoms and includes monocycles, bicycles, tricycles and spiro ring systems, wherein the ring atoms are carbon, and one to 5 ring atoms is a heteroatom such as nitrogen, sulfur or oxygen. In some embodiments, heterocyclyl includes 3- to 7-membered monocycles having one or more heteroatoms selected from nitrogen, sulfur or oxygen. In some embodiments, heterocyclyl includes 4- to 6-membered monocycles having one or more heteroatoms selected from nitrogen, sulfur or oxygen. In some embodiments, heterocyclyl includes 3-membered monocycles. In some embodiments, heterocyclyl includes 4-membered monocycles. In some embodiments, heterocyclyl includes 5-6 membered monocycles. In some embodiments, the heterocyclyl group includes 0 to 3 double bonds. In any of the foregoing embodiments, heterocyclyl includes 1, 2, 3 or 4 heteroatoms. Any nitrogen or sulfur heteroatom may optionally be oxidized (e.g., NO, SO, SO₂), and any nitrogen heteroatom may optionally be quaternized (e.g., [NR₄]⁺Cl⁻, [NR₄]⁺OH⁻). Representative examples of heterocyclyls include oxiranyl, aziridinyl, thiiranyl, azetidinyl, oxetanyl, thietanyl, 1,2-dithietanyl, 1,3-dithietanyl, pyrrolidinyl, dihydro-1H-pyrrolyl, dihydrofuranyl, tetrahydropyranyl, dihydrothienyl, tetrahydrothienyl, imidazolidinyl, piperidinyl, piperazinyl, morpholinyl, thiomorpholinyl, 1,1-dioxo-thiomorpholinyl, dihydropyranyl, tetrahydropyranyl, hexahydrothiopyranyl, hexahydropyrimidinyl, oxazinanyl, thiazinanyl, thioxanyl, homopiperazinyl, homopiperidinyl, azepanyl, oxepanyl, thiepanyl, oxazepinyl, oxazepanyl, diazepanyl, 1,4-diazepanyl, diazepinyl, thiazepinyl, thiazepanyl, tetrahydrothiopyranyl, oxazolidinyl, thiazolidinyl, isothiazolidinyl, 1,1-dioxoisothiazolidinonyl, oxazolidinonyl, imidazolidinonyl, 4,5,6,7-tetrahydro[2H]indazolyl, tetrahydrobenzoimidazolyl, 4,5,6,7-tetrahydrobenzo[d]imidazolyl, 1,6-dihydroimidazol[4,5-d]pyrrolo[2,3-b]pyridinyl, thiazinyl, thiophenyl, oxazinyl, thiadiazinyl, oxadiazinyl, dithiazinyl, dioxazinyl, oxathiazinyl, thiatriazinyl, oxatriazinyl, dithiadiazinyl, imidazolinyl, dihydropyrimidyl, tetrahydropyrimidyl, 1-pyrrolinyl, 2-pyrrolinyl, 3-pyrrolinyl, indolinyl, thiapyranyl, 2H-pyranyl, 4H-pyranyl, dioxanyl, 1,3-dioxolanyl, pyrazolinyl, pyrazolidinyl, dithianyl, dithiolanyl, pyrimidinonyl, pyrimidindionyl, pyrimidin-2,4-dionyl, piperazinonyl, piperazindionyl, pyrazolidinylimidazolinyl, 3-azabicyclo[3.1. 0]hexanyl, 3,6-diazabicyclo[3.1.1]heptanyl, 6-azabicyclo[3.1.1]heptanyl, 3-azabicyclo[3.1.1]heptanyl, 3-azabicyclo[4.1.0]heptanyl, azabicyclo[2.2.2]hexanyl, 2-azabicyclo[3.2.1]octanyl, 8-azabicyclo[3.2.1]octanyl, 2-azabicyclo[2.2.2]octanyl, 8-azabicyclo[2.2.2]octanyl, 7-oxabicyclo[2.2.1]heptane, azaspiro[3.5]nonanyl, azaspiro[2.5]octanyl, azaspiro[4.5]decanyl, 1-azaspiro[4.5]decan-2-only, azaspiro[5.5]undecanyl, tetrahydroindolyl, octahydroindolyl, tetrahydroisoindolyl, tetrahydroindazolyl, 1,1-dioxohexahydrothiopyranyl. Examples of 5-membered heterocyclyls containing a sulfur or oxygen atom and one to three nitrogen atoms are thiazolyl, including thiazol-2-yl and thiazol-2-yl N-oxide, thiadiazolyl, including 1,3,4-thiadiazol-5-yl and 1,2,4-thiadiazol-5-yl, oxazolyl, for example oxazol-2-yl, and oxadiazolyl, such as 1,3,4-oxadiazol-5-yl, and 1,2,4-oxadiazol-5-yl. Example 5-membered ring heterocyclyls containing 2 to 4 nitrogen atoms include imidazolyl, such as imidazol-2-yl; triazolyl, such as 1,3,4-triazol-5-yl; 1,2,3-triazol-5-yl, 1,2,4-triazol-5-yl, and tetrazolyl, such as 1H-tetrazol-5-yl. Representative examples of benzo-fused 5-membered heterocyclyls are benzoxazol-2-yl, benzthiazol-2-yl and benzimidazol-2-yl. Example 6-membered heterocyclyls contain one to three nitrogen atoms and optionally a sulfur or oxygen atom, for example pyridyl, such as pyrid-2-yl, pyrid-3-yl, and pyrid-4-yl; pyrimidyl, such as pyrimid-2-yl and pyrimid-4-yl; triazinyl, such as 1,3,4-triazin-2-yl and 1,3,5-triazin-4-yl; pyridazinyl, in particular pyridazin-3-yl, and pyrazinyl. The pyridine N-oxides and pyridazine N-oxides and the pyridyl, pyrimid-2-yl, pyrimid-4-yl, pyridazinyl and the 1,3,4-triazin-2-yl groups, are yet other examples of heterocyclyl groups. In some embodiments, a heterocyclic group includes a heterocyclic ring fused to one or more (e.g., 1, 2 or 3) different cyclic groups (e.g., carbocyclic rings or heterocyclic rings), where the radical or point of attachment is on the heterocyclic ring, and in some embodiments wherein the point of attachment is a heteroatom contained in the heterocyclic ring.

Thus, the term heterocyclic embraces N-heterocyclyl groups which as used herein refer to a heterocyclyl group containing at least one nitrogen and where the point of attachment of the heterocyclyl group to the rest of the molecule is through a nitrogen atom in the heterocyclyl group. Representative examples of N-heterocyclyl groups include 1-morpholinyl, 1-piperidinyl, 1-piperazinyl, 1-pyrrolidinyl, pyrazolidinyl, imidazolinyl and imidazolidinyl. The term heterocyclic also embraces C-heterocyclyl groups which as used herein refer to a heterocyclyl group containing at least one heteroatom and where the point of attachment of the heterocyclyl group to the rest of the molecule is through a carbon atom in the heterocyclyl group. Representative examples of C-heterocyclyl radicals include 2-morpholinyl, 2- or 3- or 4-piperidinyl, 2-piperazinyl, and 2- or 3-pyrrolidinyl. The term heterocyclic also embraces heterocyclylalkyl groups which as disclosed above refer to a group of the formula —R^{c}-heterocyclyl where R^{c} is an alkylene chain. The term heterocyclic also embraces heterocyclylalkoxy groups which as used herein refer to a radical bonded through an oxygen atom of the formula —O—R^{c}-heterocyclyl where R^{c} is an alkylene chain.

As used herein, the term “heteroaryl” used alone or as part of a larger moiety (e.g., “heteroarylalkyl” (also “heteroaralkyl”), or “heteroarylalkoxy” (also “heteroaralkoxy”), refers to a monocyclic, bicyclic or tricyclic ring system having 5 to 14 ring atoms, wherein at least one ring is aromatic and contains at least one heteroatom. In one embodiment, heteroaryl includes 5-6 membered monocyclic aromatic groups where one or more ring atoms is nitrogen, sulfur or oxygen. Representative examples of heteroaryl groups include thienyl, furyl, imidazolyl, pyrazolyl, thiazolyl, isothiazolyl, oxazolyl, isoxazolyl, triazolyl, thiadiazolyl, oxadiazolyl, tetrazolyl, thiatriazolyl, oxatriazolyl, pyridyl, pyrimidyl, imidazopyridyl, pyrazinyl, pyridazinyl, triazinyl, tetrazinyl, tetrazolo[1,5-b]pyridazinyl, purinyl, deazapurinyl, benzoxazolyl, benzofuryl, benzothiazolyl, benzothiadiazolyl, benzotriazolyl, benzoimidazolyl, indolyl, 1,3-thiazol-2-yl, 1,3,4-triazol-5-yl, 1,3-oxazol-2-yl, 1,3,4-oxadiazol-5-yl, 1,2,4-oxadiazol-5-yl, 1,3,4-thiadiazol-5-yl, 1H-tetrazol-5-yl, 1,2,3-triazol-5-yl, and pyrid-2-yl N-oxide. The term “heteroaryl” also includes groups in which a heteroaryl is fused to one or more cyclic (e.g., carbocyclyl, or heterocyclyl) rings, where the radical or point of attachment is on the heteroaryl ring. Nonlimiting examples include indolyl, indolizinyl, isoindolyl, benzothienyl, benzothiophenyl, methylenedioxyphenyl, benzofuranyl, dibenzofuranyl, indazolyl, benzimidazolyl, benzodioxazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl and pyrido[2,3-b]-1,4-oxazin-3(4H)-one. A heteroaryl group may be mono-, bi- or tri-cyclic. In some embodiments, a heteroaryl group includes a heteroaryl ring fused to one or more (e.g., 1, 2 or 3) different cyclic groups (e.g., carbocyclic rings or heterocyclic rings), where the radical or point of attachment is on the heteroaryl ring, and in some embodiments wherein the point of attachment is a heteroatom contained in the heterocyclic ring.

Thus, the term heteroaryl embraces N-heteroaryl groups which as used herein refer to a heteroaryl group as defined above containing at least one nitrogen and where the point of attachment of the heteroaryl group to the rest of the molecule is through a nitrogen atom in the heteroaryl group. The term heteroaryl also embraces C-heteroaryl groups which as used herein refer to a heteroaryl group as defined above and where the point of attachment of the heteroaryl group to the rest of the molecule is through a carbon atom in the heteroaryl group. The term heteroaryl also embraces heteroarylalkyl groups which as disclosed above refer to a group of the formula —W-heteroaryl, wherein R^{c} is an alkylene chain as defined above. The term heteroaryl also embraces heteroaralkoxy (or heteroarylalkoxy) groups which as used herein refer to a group bonded through an oxygen atom of the formula —O—R^{c}-heteroaryl, where R^{c} is an alkylene group as defined above.

Any of the groups described herein may be substituted or unsubstituted. As used herein, the term “substituted” broadly refers to all permissible substituents with the implicit proviso that such substitution is in accordance with permitted valence of the substituted atom and the substituent, and that the substitution results in a stable compound, i.e. a compound that does not spontaneously undergo transformation such as by rearrangement, cyclization, elimination, etc. Representative substituents include halogens, hydroxyl groups, and any other organic groupings containing any number of carbon atoms, e.g., 1-14 carbon atoms, and which may include one or more (e.g., 1, 2, 3, or 4) heteroatoms such as oxygen, sulfur, and nitrogen grouped in a linear, branched, or cyclic structural format.

To the extent not disclosed otherwise for any particular group(s), representative examples of substituents may include alkyl, substituted alkyl (e.g., C₁-C₆, C₁-C₅, C₁-C₄, C₁-C₃, C₁-C₂, alkoxy (e.g., C₁-C₆, C₁-C₅, C₁-C₄, C₁-C₃, C₁-C₂, C₁), substituted alkoxy (e.g., C₁-C₆, C₁-C₅, C₁-C₄, C₁-C₃, C₁-C₂, C₁), haloalkyl (e.g., CF₃), alkenyl (e.g., C₂-C₆, C₂-C₅, C₂-C₄, C₂-C₃, C₂), substituted alkenyl (e.g., C₂-C₆, C₂-C₅, C₂-C₄, C₂-C₃, C₂), alkynyl (e.g., C₂-C₆, C₂-C₅, C₂-C₄, C₂-C₃, C₂), substituted alkynyl (e.g., C₂-C₆, C₂-C₅, C₂-C₄, C₂-C₃, C₂), cyclic (e.g., C₃-C₁₂, C₅-C₆), substituted cyclic (e.g., C₃-C₁₂, C₅-C₆), carbocyclic (e.g., C₃-C₁₂, C₅-C₆), substituted carbocyclic (e.g., C₃-C₁₂, C₅-C₆), heterocyclic (e.g., C₃-C₁₂, C₅-C₆), substituted heterocyclic (e.g., C₃-C₁₂, C₅-C₆), aryl (e.g., benzyl and phenyl), substituted aryl (e.g., substituted benzyl or phenyl), heteroaryl (e.g., pyridyl or pyrimidyl), substituted heteroaryl (e.g., substituted pyridyl or pyrimidyl), aralkyl (e.g., benzyl), substituted aralkyl (e.g., substituted benzyl), halo, hydroxyl, aryloxy (e.g., C₆-C₁₂, C₆), substituted aryloxy (e.g., C₆-C₁₂, C₆), alkylthio (e.g., C₁-C₆), substituted alkylthio (e.g., C₁-C₆), arylthio (e.g., C₆-C₁₂, C₆), substituted arylthio (e.g., C₆-C₁₂, C₆), cyano, carbonyl, substituted carbonyl, carboxyl, substituted carboxyl, amino, substituted amino, amido, substituted amido, thio, substituted thio, sulfinyl, substituted sulfinyl, sulfonyl, substituted sulfonyl, sulfinamide, substituted sulfinamide, sulfonamide, substituted sulfonamide, urea, substituted urea, carbamate, substituted carbamate, amino acid, and peptide groups.

The term “binding” as it relates to interaction between the targeting ligand and the targeted protein, which in this case is tau protein and mutant or misfolded forms thereof, refers to an inter-molecular interaction that is substantially specific in that binding of the targeting ligand with other proteinaceous entities present in the cell may be functionally insignificant. The present bispecific compounds bind and recruit tau protein for selective degradation.

The term “binding” as it relates to interaction between the degron and the E3 ubiquitin ligase, typically refers to an inter-molecular interaction that may or may not exhibit an affinity level that equals or exceeds that affinity between the targeting ligand and the target protein, but nonetheless wherein the affinity is sufficient to achieve recruitment of the ligase to the targeted degradation and the selective degradation of the targeted protein.

Broadly, the bispecific compounds of the present invention have a structure represented by formula (I):

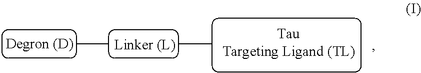

wherein the targeting ligand represents a moiety that binds tau, the degron represents a moiety that binds an E3 ubiquitin ligase or the degron is an autophagy-recruiting tag (i.e. a tag that destines substrates for selective autophagy), and the linker represents a moiety that covalently connects the degron and the targeting ligand, or a pharmaceutically acceptable salt or stereoisomer thereof,
wherein

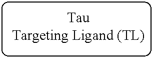

is represented by the formula TL-1 or TL-2:

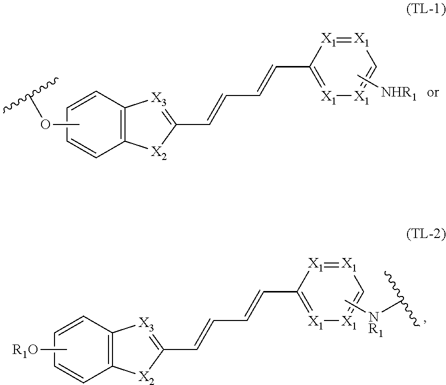

wherein
each X₁ is independently C, CH, or N;

### X₂ is NH, S, or O;

### X₃ is CH or N; and

each R₁ is independently hydrogen or C₁-C₃ alkyl,
provided that when X₂ is NH, X₃ is CH; and
provided that when X₃ is N, X₂ is S or O.

Thus, in some embodiments, the bispecific compounds of the present invention have a structure as represented by formula I-1:

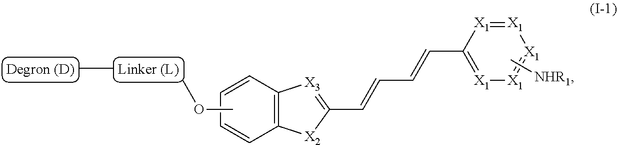

or a pharmaceutically acceptable salt or stereoisomer thereof.

Thus, in other embodiments, the bispecific compounds of the present invention have a structure as represented by formula I-2:

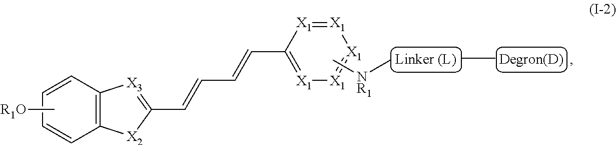

or a pharmaceutically acceptable salt or stereoisomer thereof.

In some embodiments, the formula of TL-1 is TL-1a to TL-1o:

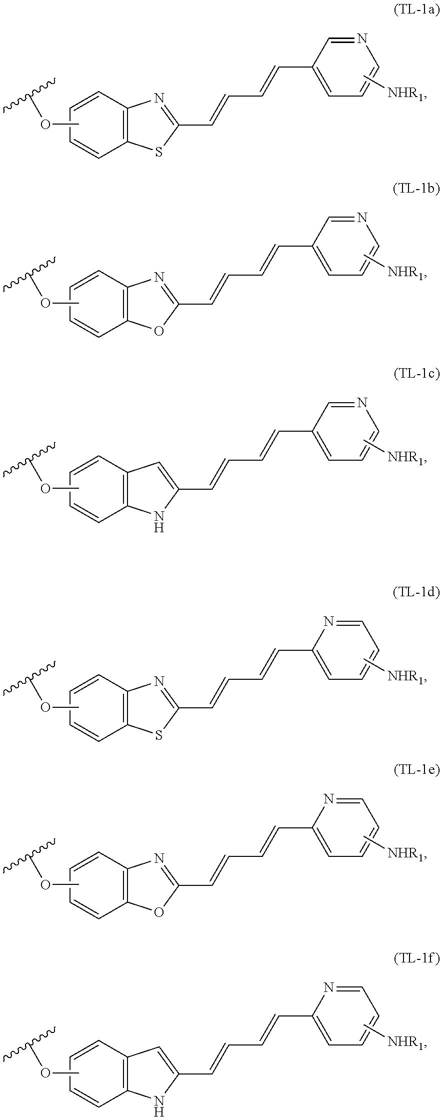

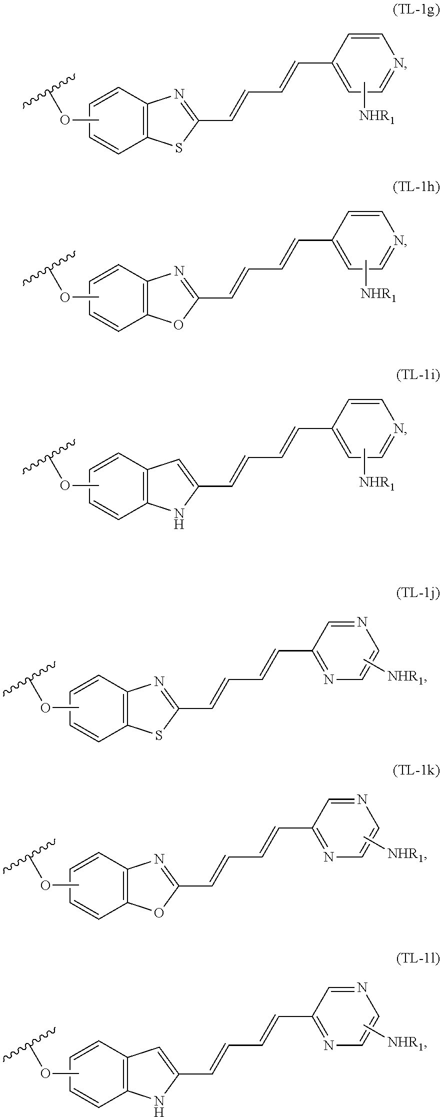

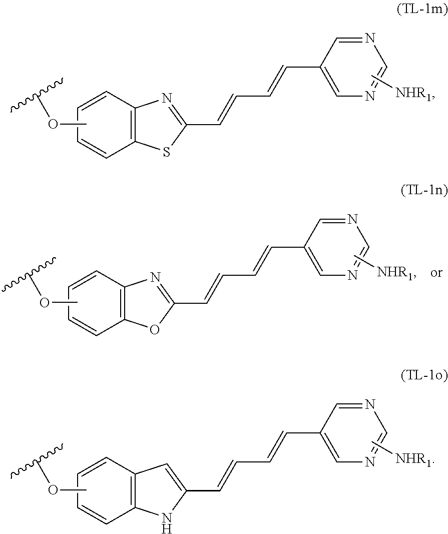

In some embodiments, TL-1 is

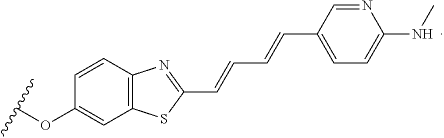 [SMILES: *Oc1ccc2nc(/C=C/C=C/c3ccc(NC)nc3)sc2c1 ; "."]

In some embodiments, the formula of TL-2 is TL-2a to TL-2o:

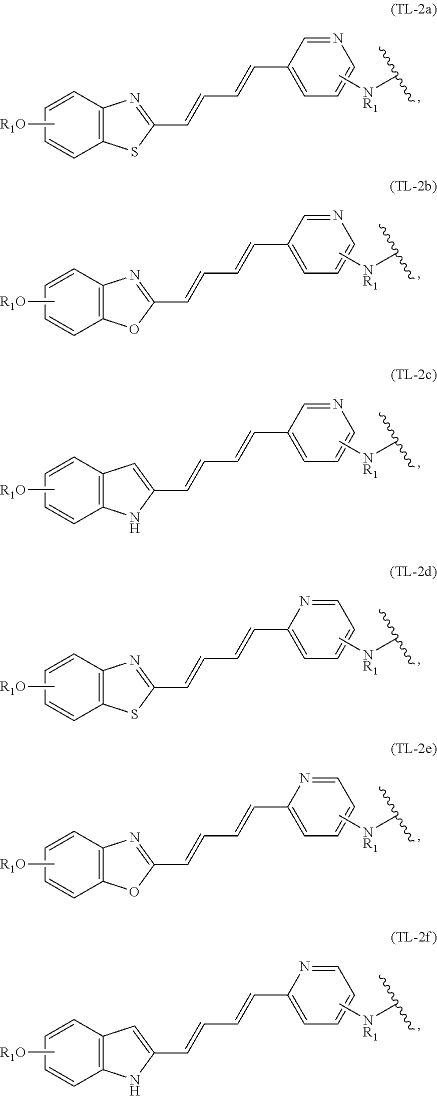

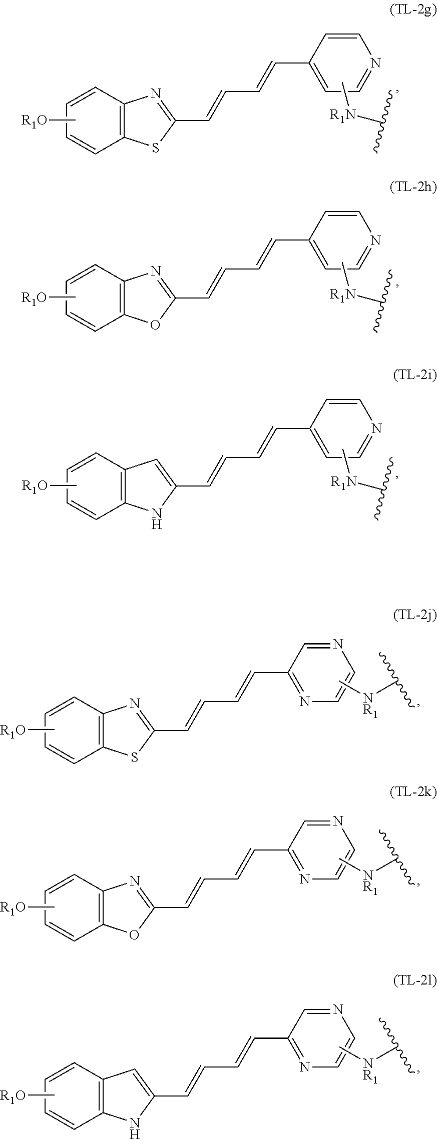

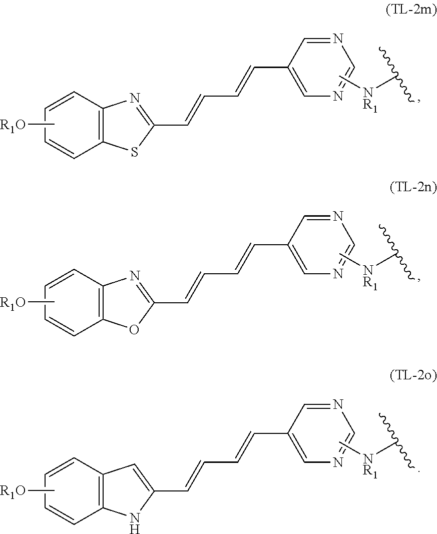

In some embodiments, R₁ is hydrogen.

In some embodiments, R₁ is methyl.

### Linkers

The linker (“L”) provides a covalent attachment between the targeting ligand and the degron. The structure of linker may not be critical, provided it is substantially non-interfering with the activity of the Tau targeting ligand or the degron. In some embodiments, the linker includes an alkylene chain (e.g., having 1-20 alkylene units). In other embodiments, the linker may include an alkylene chain or a bivalent alkylene chain, either of which may be interrupted by, and/or terminate (at either or both termini) at least one of —O—, —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, C₃-C₁₂ carbocyclene, 3- to 12-membered heterocyclene, 5- to 12-membered heteroarylene or any combination thereof, wherein R′ is H or C₁-C₆ alkyl, wherein the interrupting and the one or both terminating groups may be the same or different.

In some embodiments, the alkylene chain has 1-18 alkylene units. In some embodiments, the alkylene chain has 1-12 alkylene units. In some embodiments, the alkylene chain has 1-10 alkylene units. In some embodiments, the alkylene chain has 1-8 alkylene units. In some embodiments, the alkylene chain has 1-6 alkylene units. In some embodiments, the alkylene chain has 1˜4 alkylene units. In some embodiments, the alkylene chain has 1-2 alkylene units. In some embodiments, the alkylene chain is interrupted by, and/or terminates (at either or both termini) in at least one of —N(R′)—, —C(O)—, —C(O)O—, —OC(O)—, —C(O)N(R′)—, —N(R′)C(O)—, —N(R′)C(O)O—, —OC(O)N(R′)—, —S(O)₂—, —N(R′)S(O)₂—, —S(O)₂N(R′)—, or a combination thereof. In some embodiments, the alkylene chain is interrupted by, and/or terminates (at either or both termini) with —N(R′)—. In some embodiments, the alkylene chain is interrupted by, and/or terminates (at either or both termini) with —C(O)—. In some embodiments, the alkylene chain is interrupted by, and/or terminates (at either or both termini) with —C(O)O—. In some embodiments, the alkylene chain is interrupted by, and/or terminates (at either or both termini) with —C(O)N(R′)—. In some embodiments, the alkylene chain is interrupted by, and/or terminates (at either or both termini) with —N(R)S(O)₂—

In some embodiments, the linker includes an alkylene chain having 1-10 alkylene units and interrupted by or terminating in

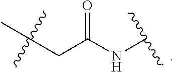 [SMILES: *CC(=O)N*]

“Carbocyclene” refers to a bivalent carbocycle radical, which is optionally substituted.

“Heterocyclene” refers to a bivalent heterocyclyl radical which may be optionally substituted.

“Heteroarylene” refers to a bivalent heteroaryl radical which may be optionally substituted.

Representative examples of alkylene linkers that may be suitable for use in the present invention include the following:

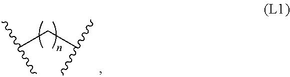

wherein n is an integer of 1-12 (“of” meaning inclusive), e.g., 1-12, 1-11, 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, 9-10 and 1, 2, 3, 4, 5, 6, 7, 8, 9 and 10, examples of which include:

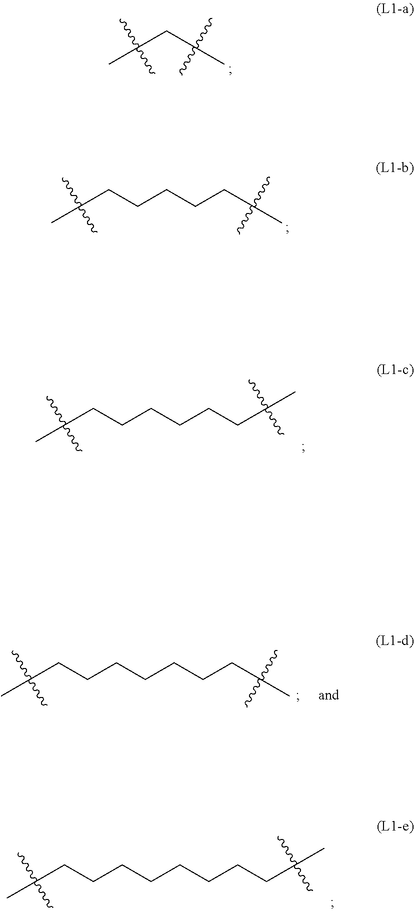

alkylene chains terminating in various functional groups (as described above), examples of which are as follows:

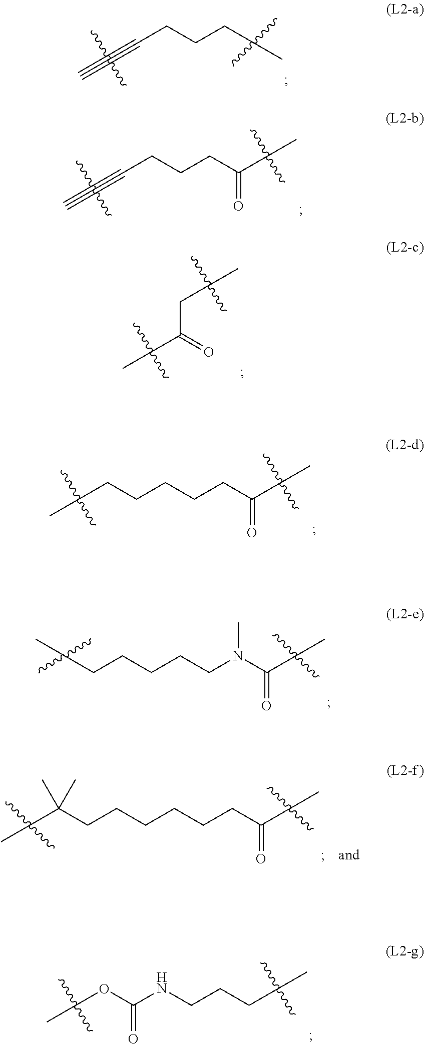

alkylene chains interrupted with various functional groups (as described above), examples of which are as follows:

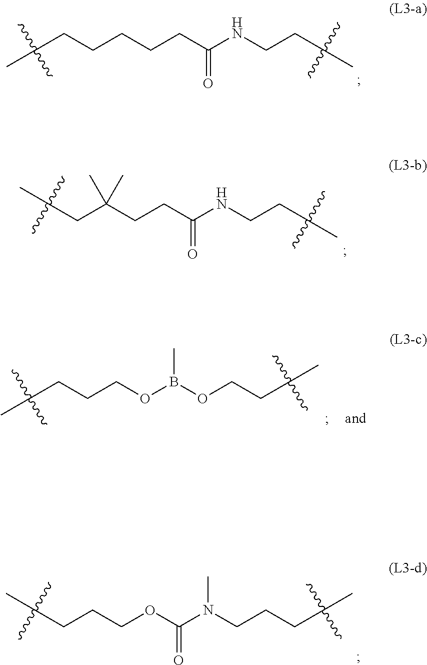 [SMILES: *CCCCCC(=O)NCC* ; *CCNC(=O)CCC(C)(C)C* ; *CCCOB(C)OCC* ; *CCCOC(=O)N(C)CCC*]

alkylene chains interrupted or terminating with heterocyclene groups, e.g.,

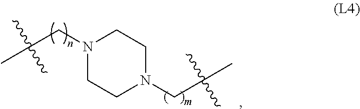

wherein m and n are independently integers of 0-10, examples of which include:

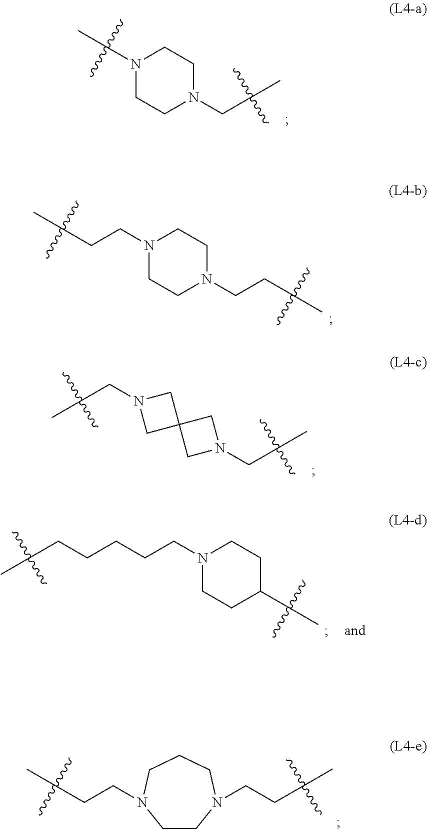 [SMILES: *CN1CCN(*)CC1 ; *CCN1CCN(CC*)CC1 ; *CN1CC2(C1)CN(C*)C2 ; *CCCCCN1CCC(*)CC1 ; *CCN1CCCN(CC*)CC1]

alkylene chains interrupted by amide, heterocyclene and/or aryl groups, examples of which include:

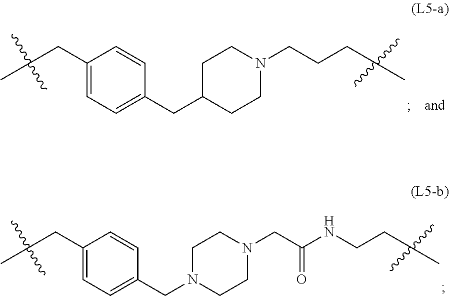 [SMILES: *CCCN1CCC(Cc2ccc(C*)cc2)CC1 ; *CCNC(=O)CN1CCN(Cc2ccc(C*)cc2)CC1]

alkylene chains interrupted by heterocyclene and aryl groups, and a heteroatom, examples of which include:

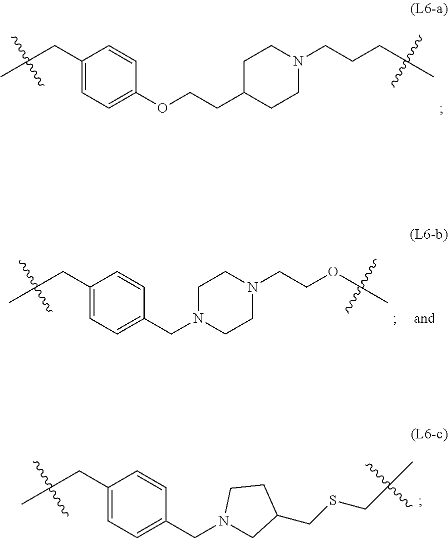 [SMILES: *CCCN1CCC(CCOc2ccc(C*)cc2)CC1 ; *Cc1ccc(CN2CCN(CCO*)CC2)cc1 ; *CSCC1CCN(Cc2ccc(C*)cc2)C1]

and
alkylene chains interrupted by a heteroatom such as N, O or B, e.g.,

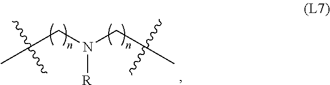

wherein each n is independently an integer of 1-10, e.g., 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, 9-10, and 1, 2, 3, 4, 5, 6, 7, 8, 9 and 10, and R is H or C₁ to C₄ alkyl, an example of which is

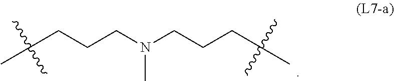 [SMILES: *CCCN(C)CCC*]

In some embodiments, the linker may include a polyethylene glycol chain which may terminate (at either or both termini) in at least one of —S—, —C(O)—, —C(O)O—, —OC(O)—, —OC(O)O—, —C(NOR′)—, —C(O)N(R′)—, —C(O)N(R′)C(O)—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, C₃₋₁₂ carbocyclene, 3- to 12-membered heterocyclene, 5- to 12-membered heteroarylene or any combination thereof, wherein R′ is H or C₁-C₆ alkyl, wherein the one or both terminating groups may be the same or different.

In some embodiments, the polyethylene glycol chain has 1 to 10 —(CH₂CH₂—O)— units. In some embodiments, the polyethylene glycol chain has 1 to 5 —(CH₂CH₂—O)— units. In some embodiments, the polyethylene glycol chain has 1 to 2 —(CH₂CH₂—O)— units. In some embodiments, the polyethylene glycol is interrupted by, and/or terminates (at either or both termini) in at least one of —N(R′)—, —C(O)—, —C(O)O—, —OC(O)—, —C(O)N(R′)—, —N(R′)C(O)—, —N(R′)C(O)O—, —OC(O)N(R′)—, —S(O)₂—, —N(R′)S(O)₂—, —S(O)₂N(R′)—, or a combination thereof. In some embodiments, the polyethylene glycol chain is interrupted by, and/or terminates (at either or both termini) with —N(R′)—. In some embodiments, the polyethylene glycol chain is interrupted by, and/or terminates (at either or both termini) with —C(O)—. In some embodiments, the polyethylene glycol chain is interrupted by, and/or terminates (at either or both termini) with —C(O)O—. In some embodiments, the polyethylene glycol chain is interrupted by, and/or terminates (at either or both termini) with —C(O)N(R′)—. In some embodiments, the polyethylene glycol chain is interrupted by, and/or terminates (at either or both termini) with —N(R′)S(O)₂—.

In some embodiments, the linker includes a polyethylene glycol chain having 2-8 PEG units and terminating in

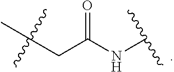 [SMILES: *CC(=O)N*]

Examples of linkers that include a polyethylene glycol chain include:

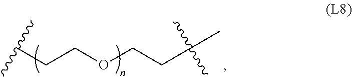

wherein n is an integer of 1-10, examples of which include:

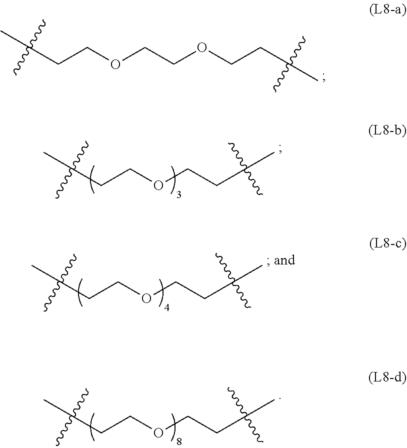

In some embodiments, the polyethylene glycol linker may terminate in a functional group, examples of which are as follows:

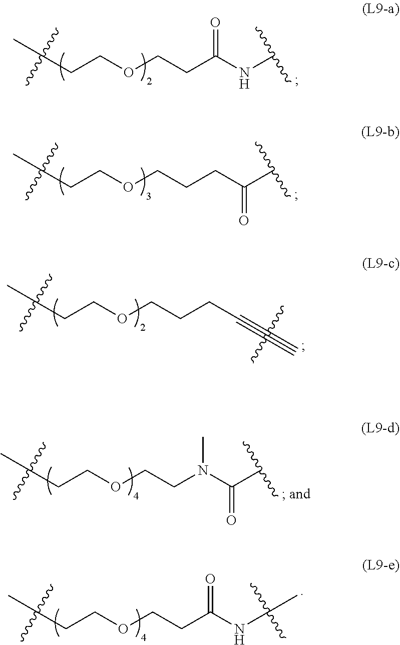

In some embodiments, the bispecific compound of formula (I) includes a linker that is represented by any one of the following structures:

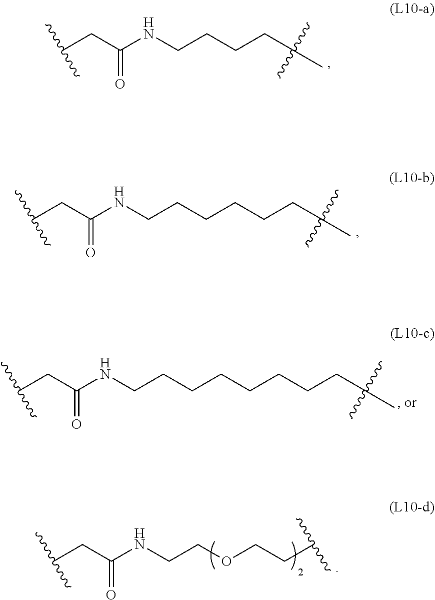

Thus, in some embodiments, the bispecific compounds of the present invention are represented by any one of the following structures:

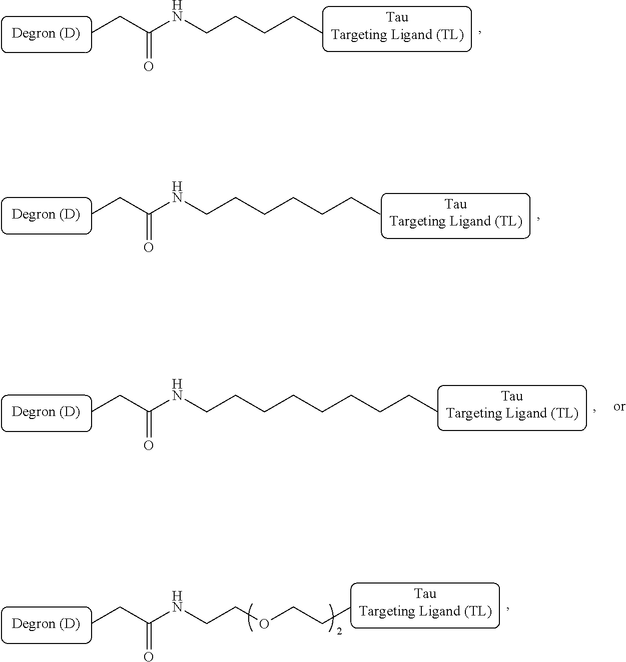

or a pharmaceutically acceptable salt or stereoisomer thereof.

In some embodiments, the bispecific compounds of the present invention are represented by any one of the following structures:

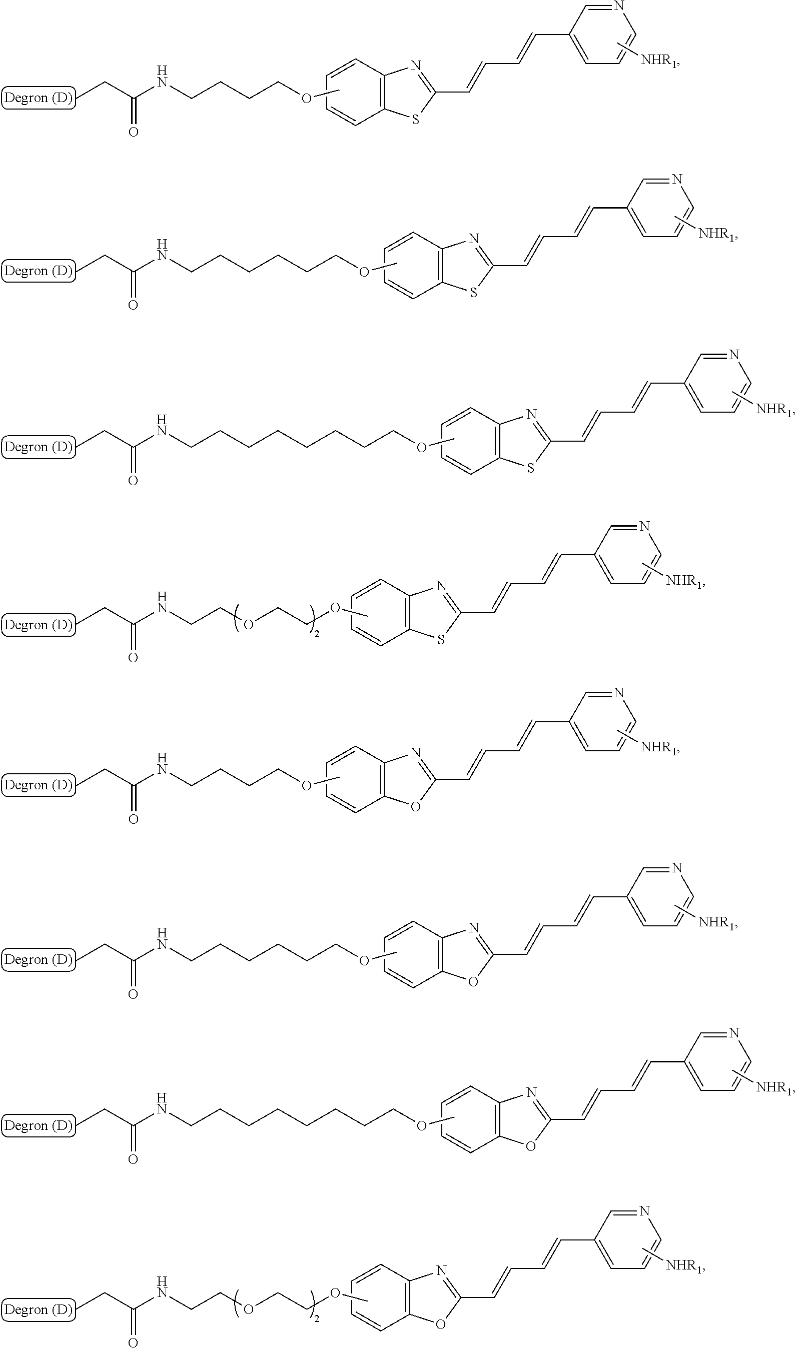

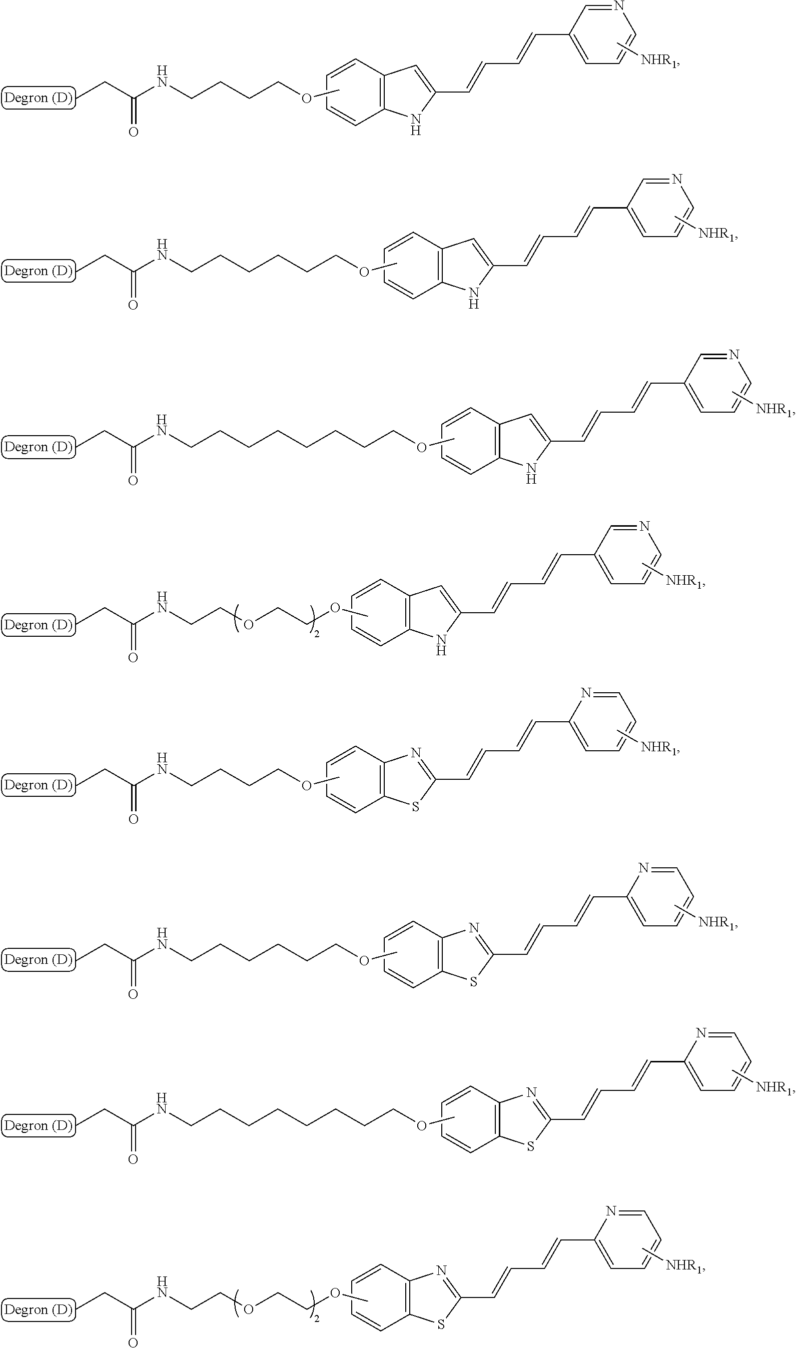

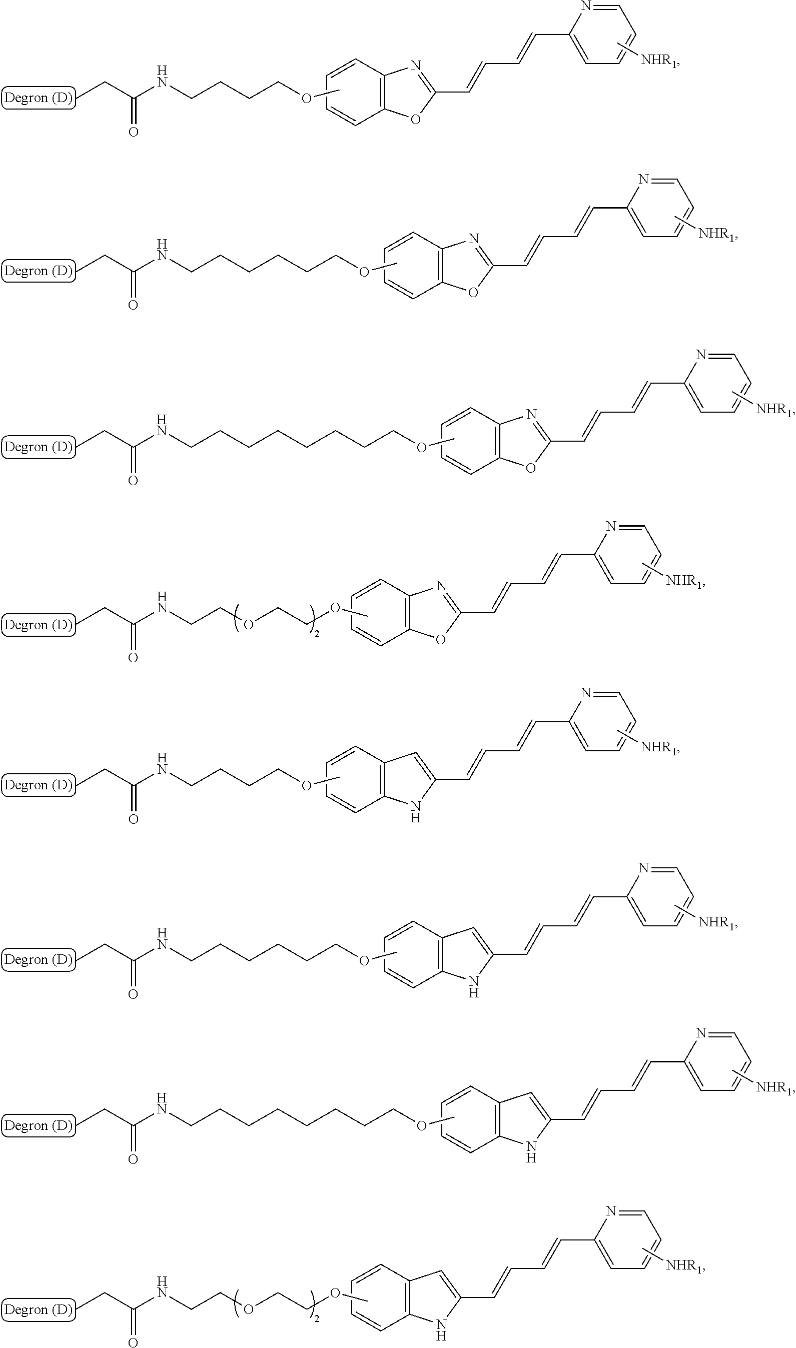

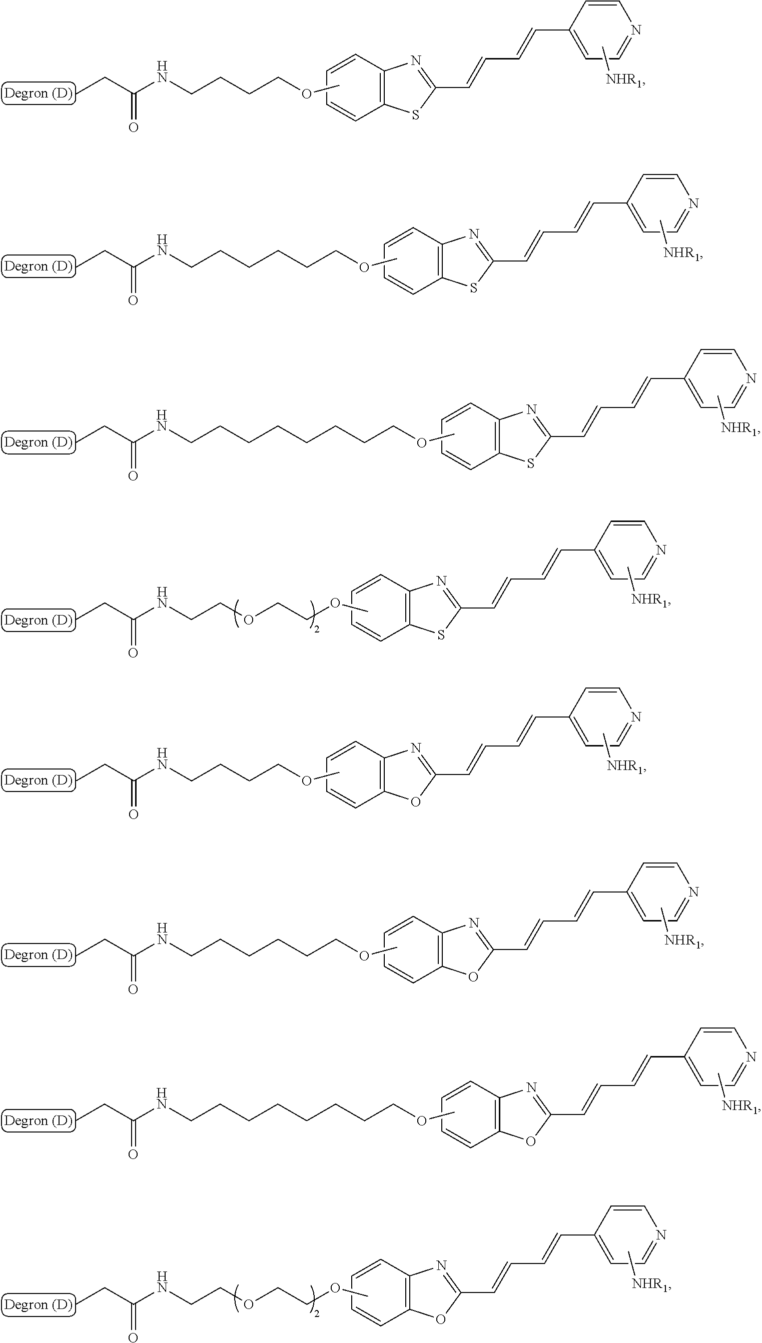

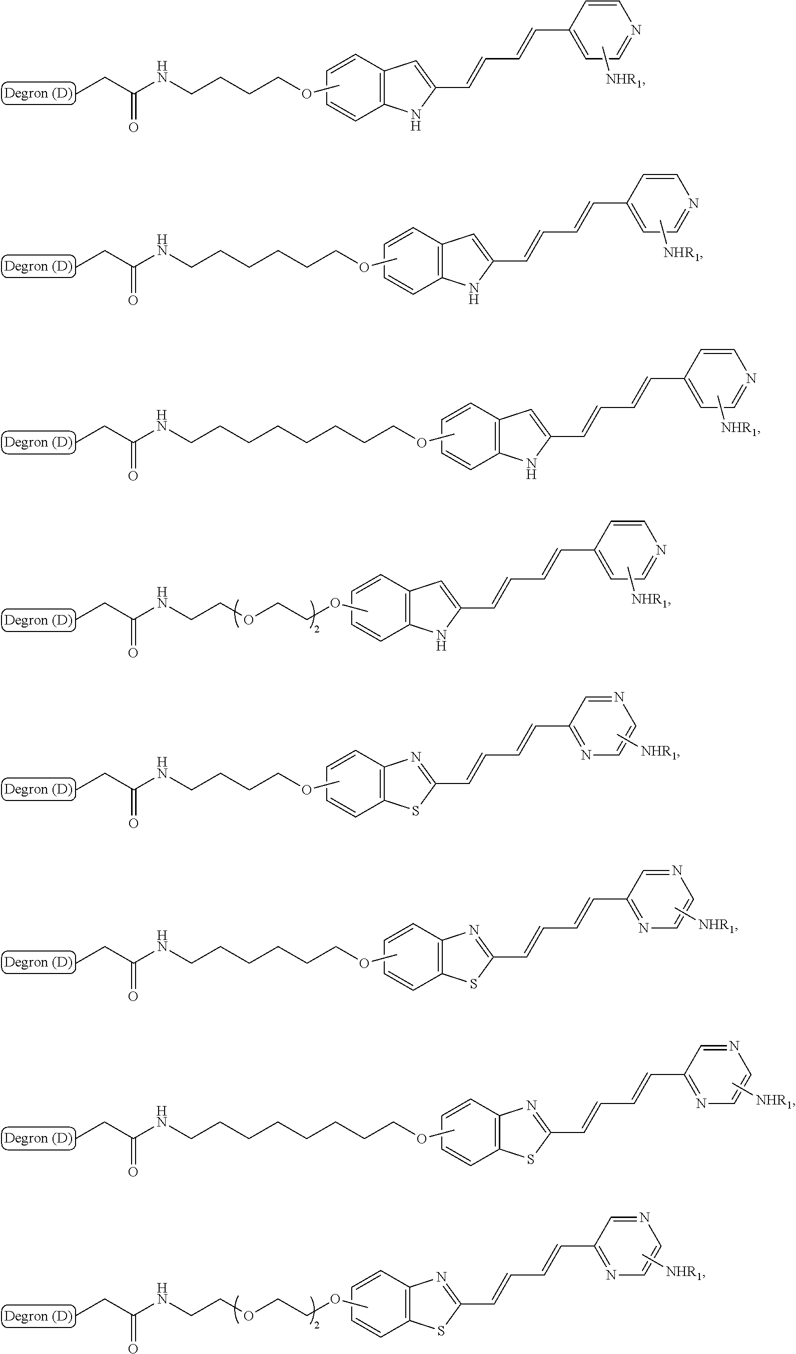

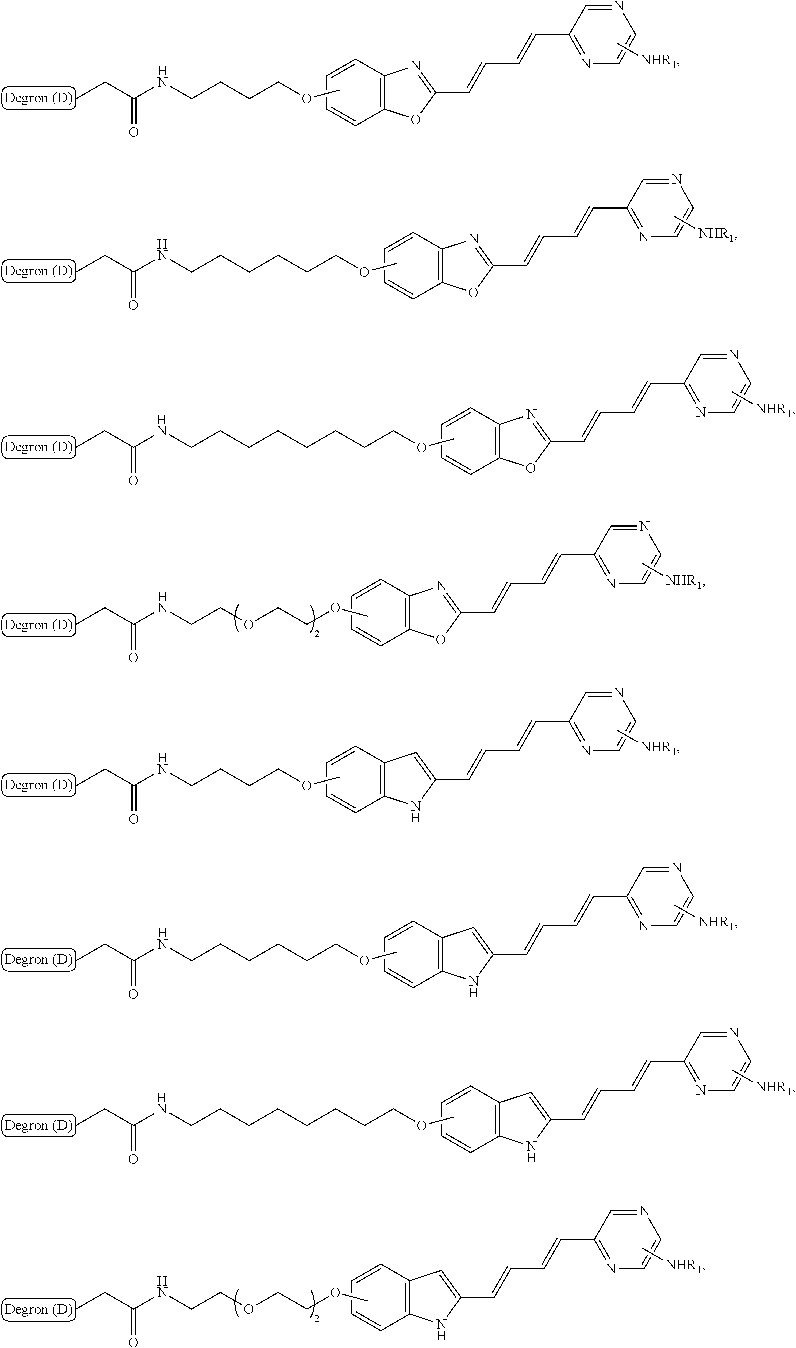

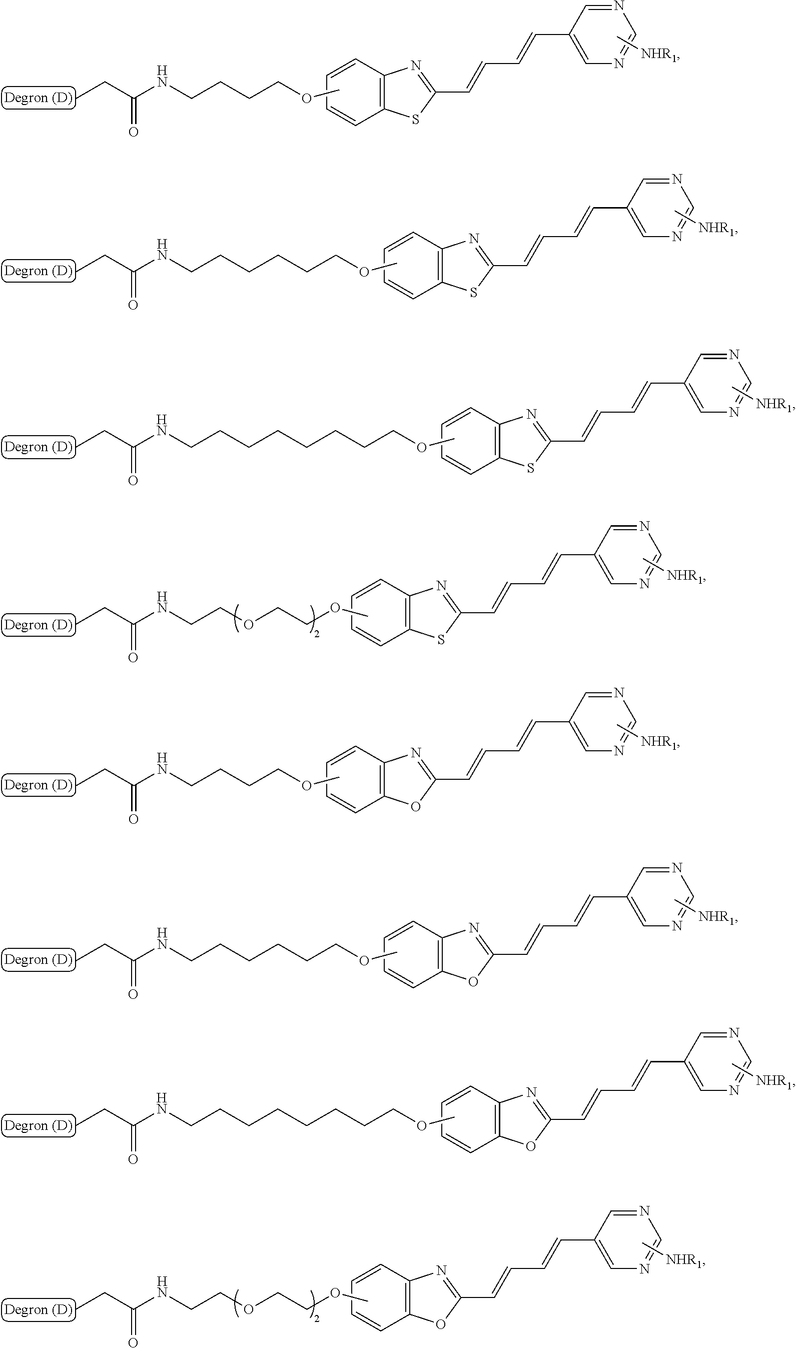

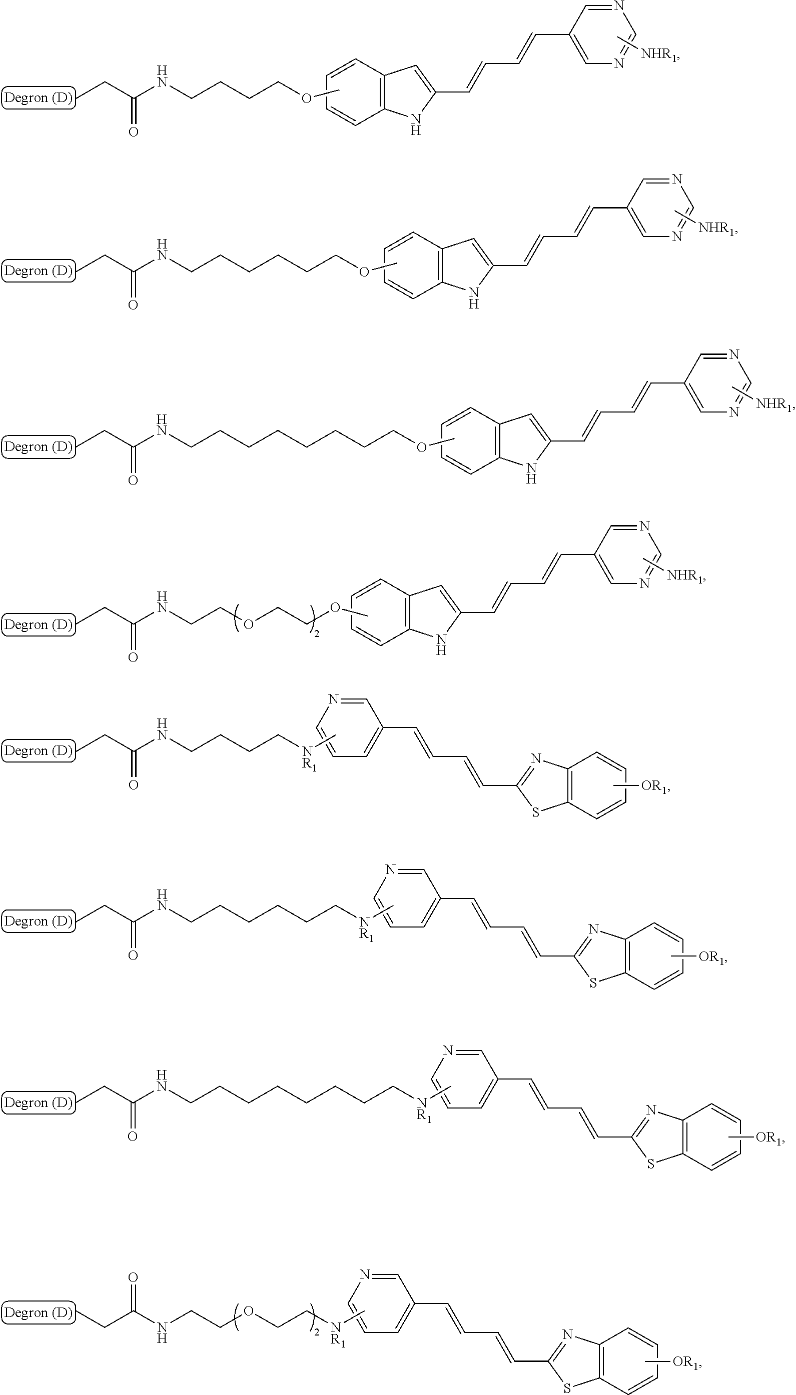

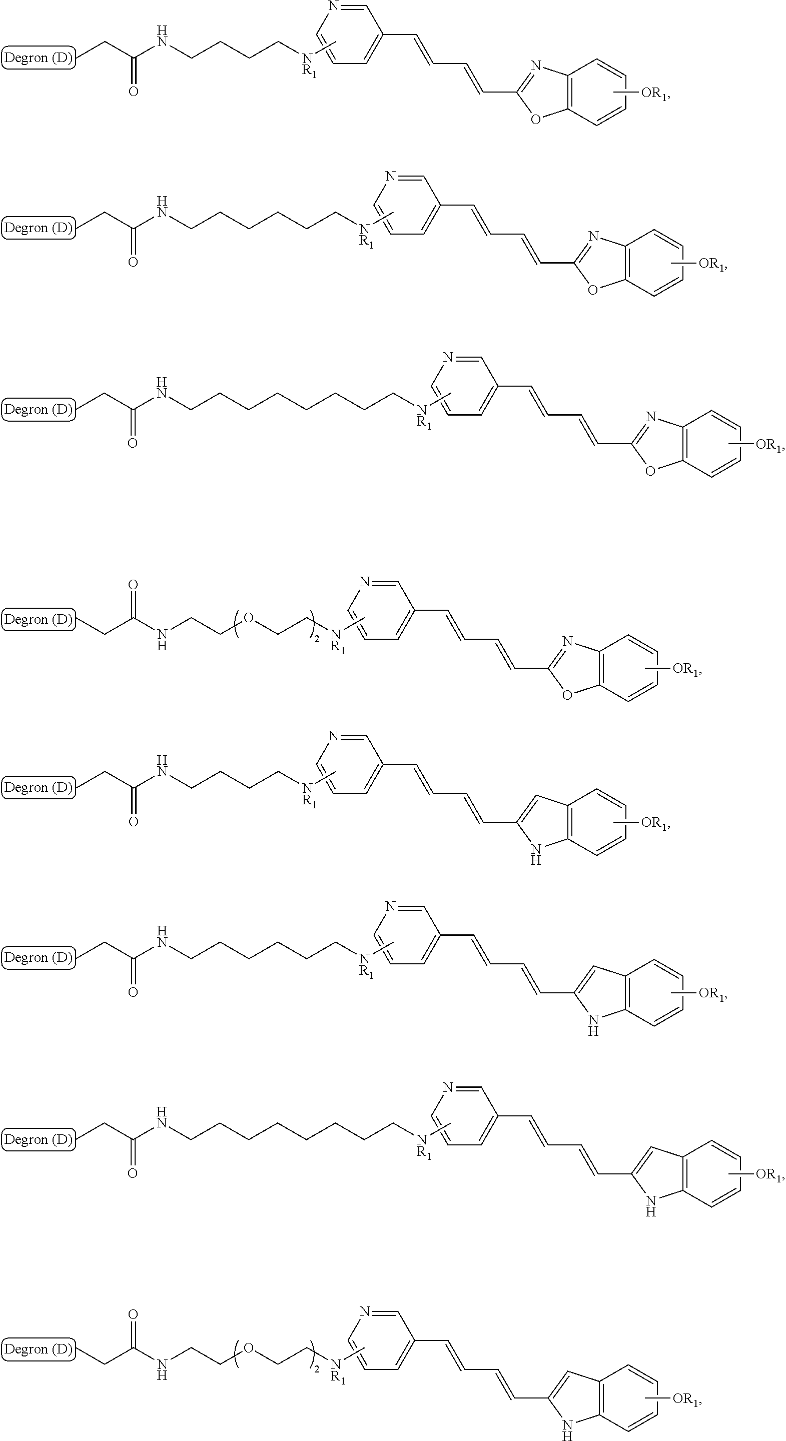

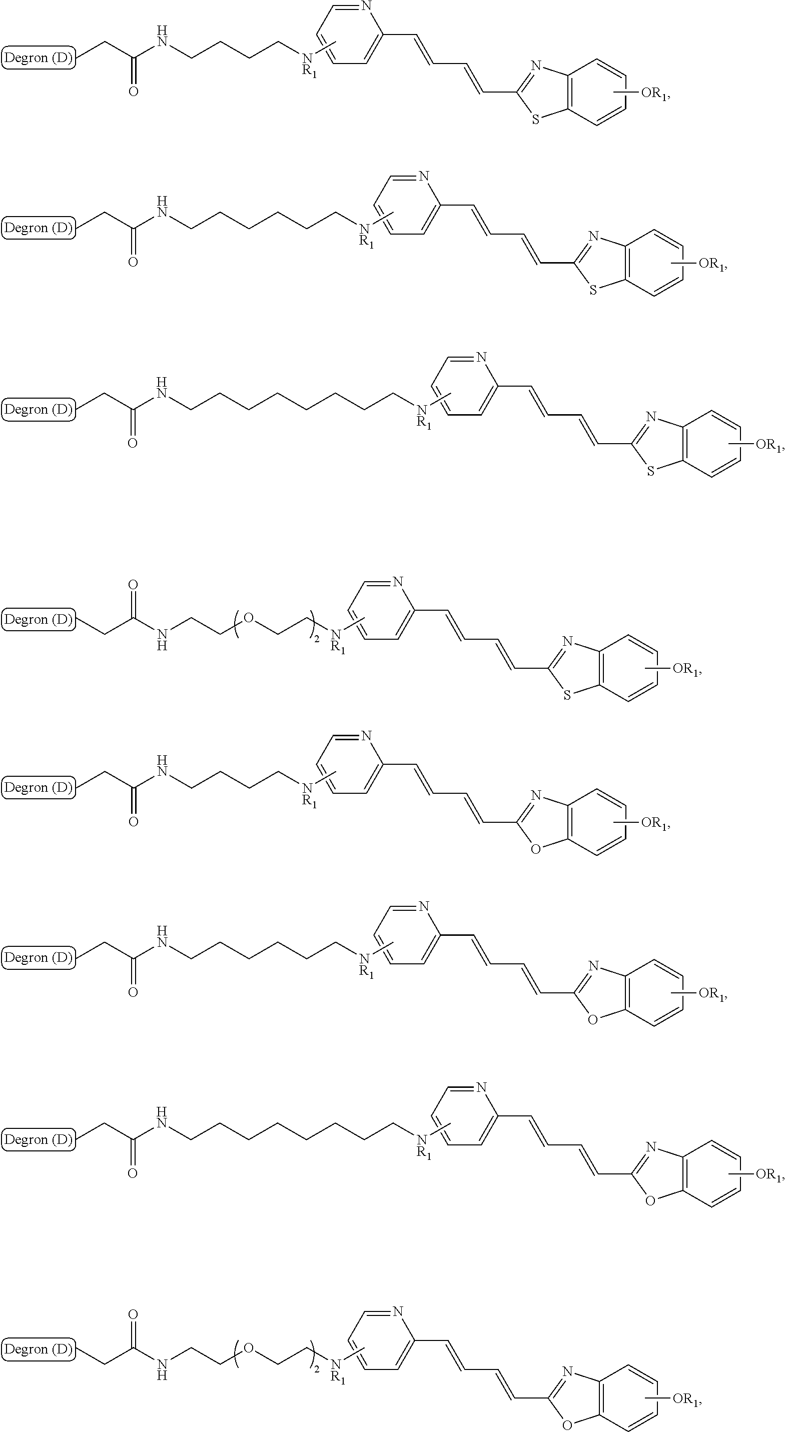

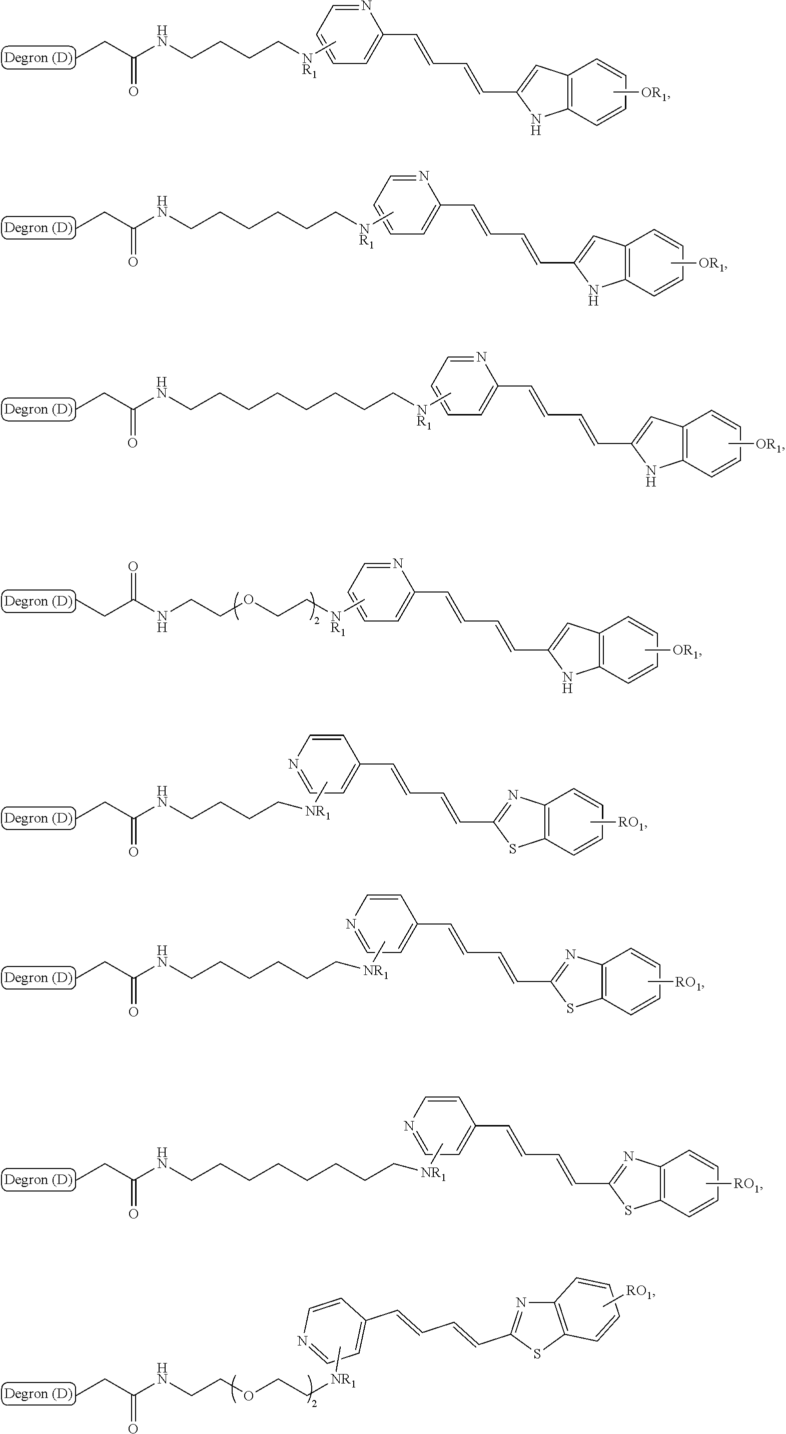

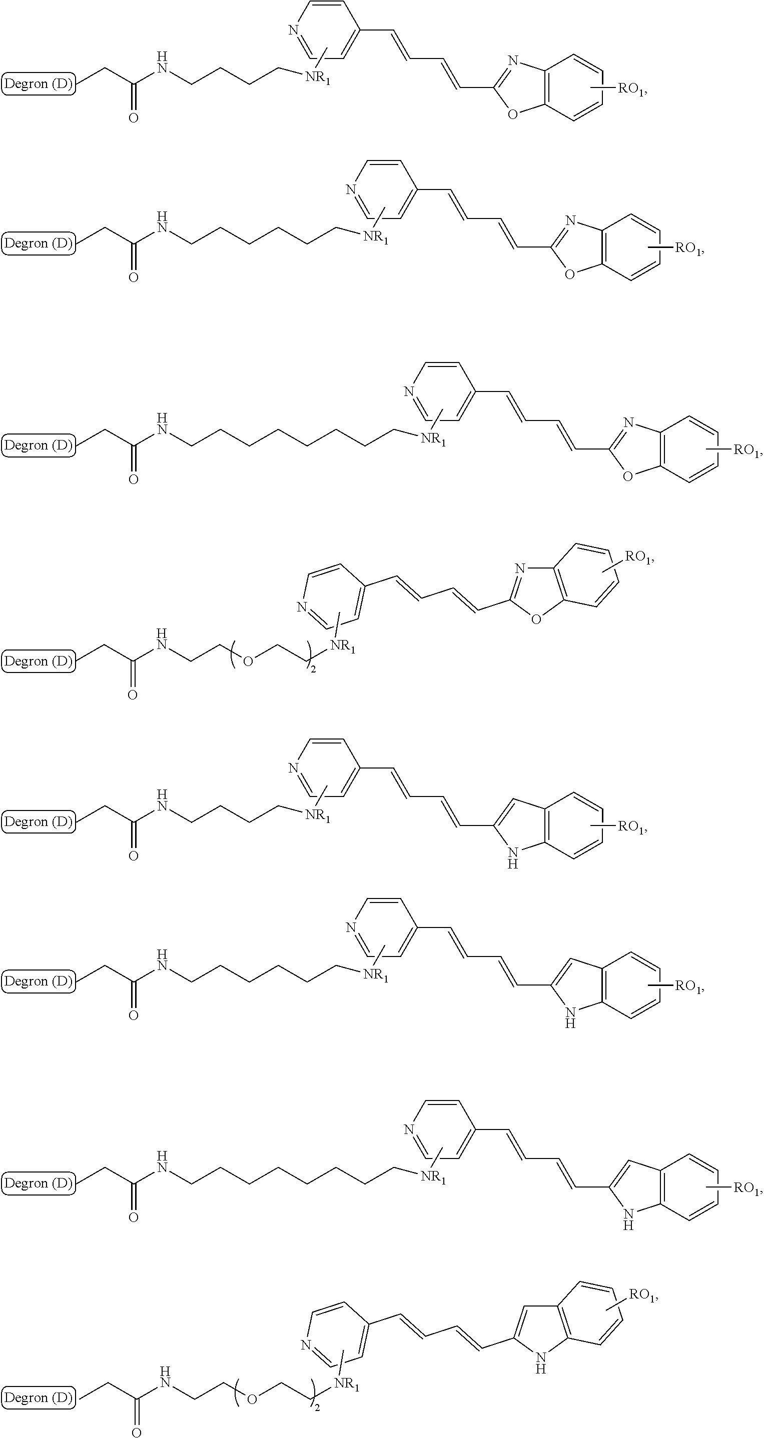

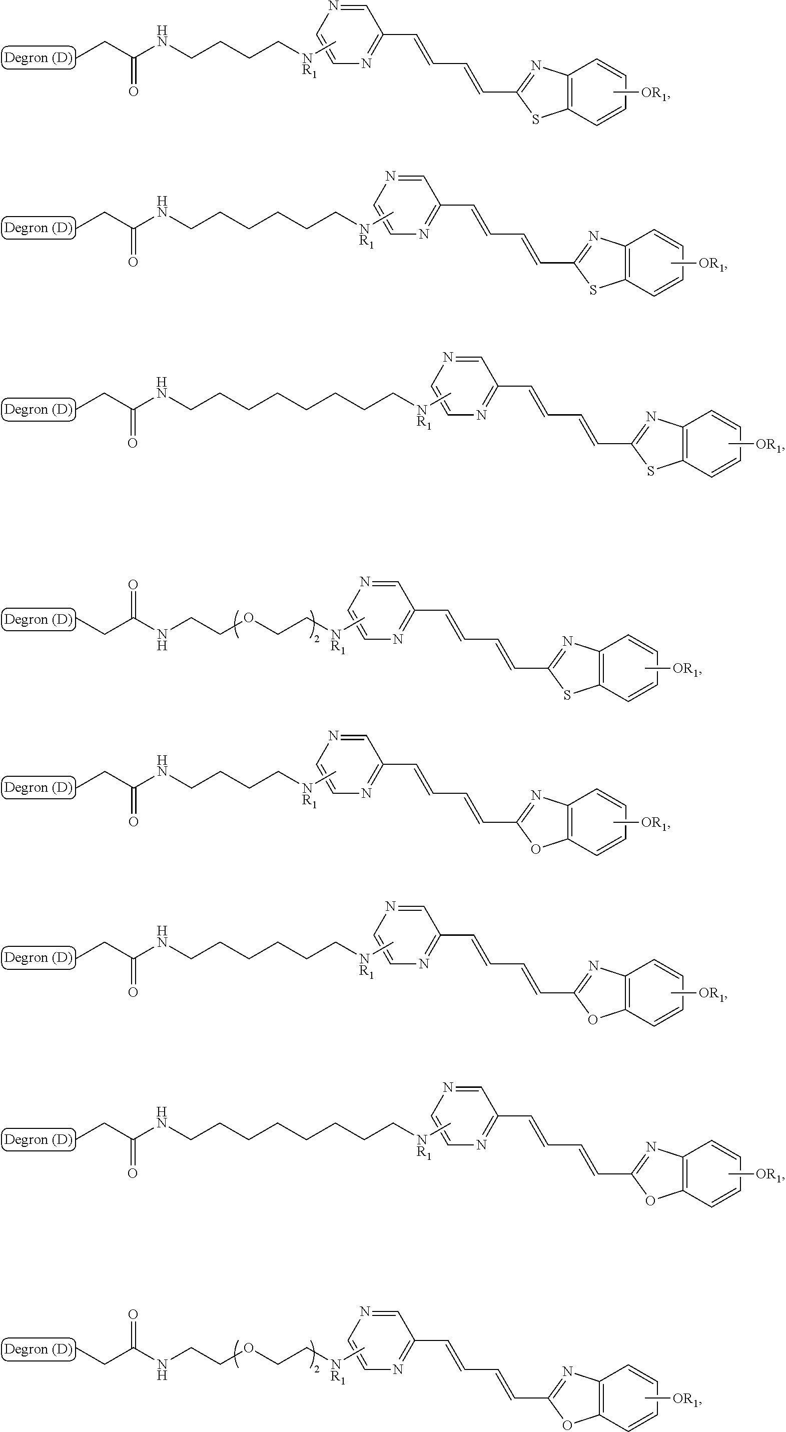

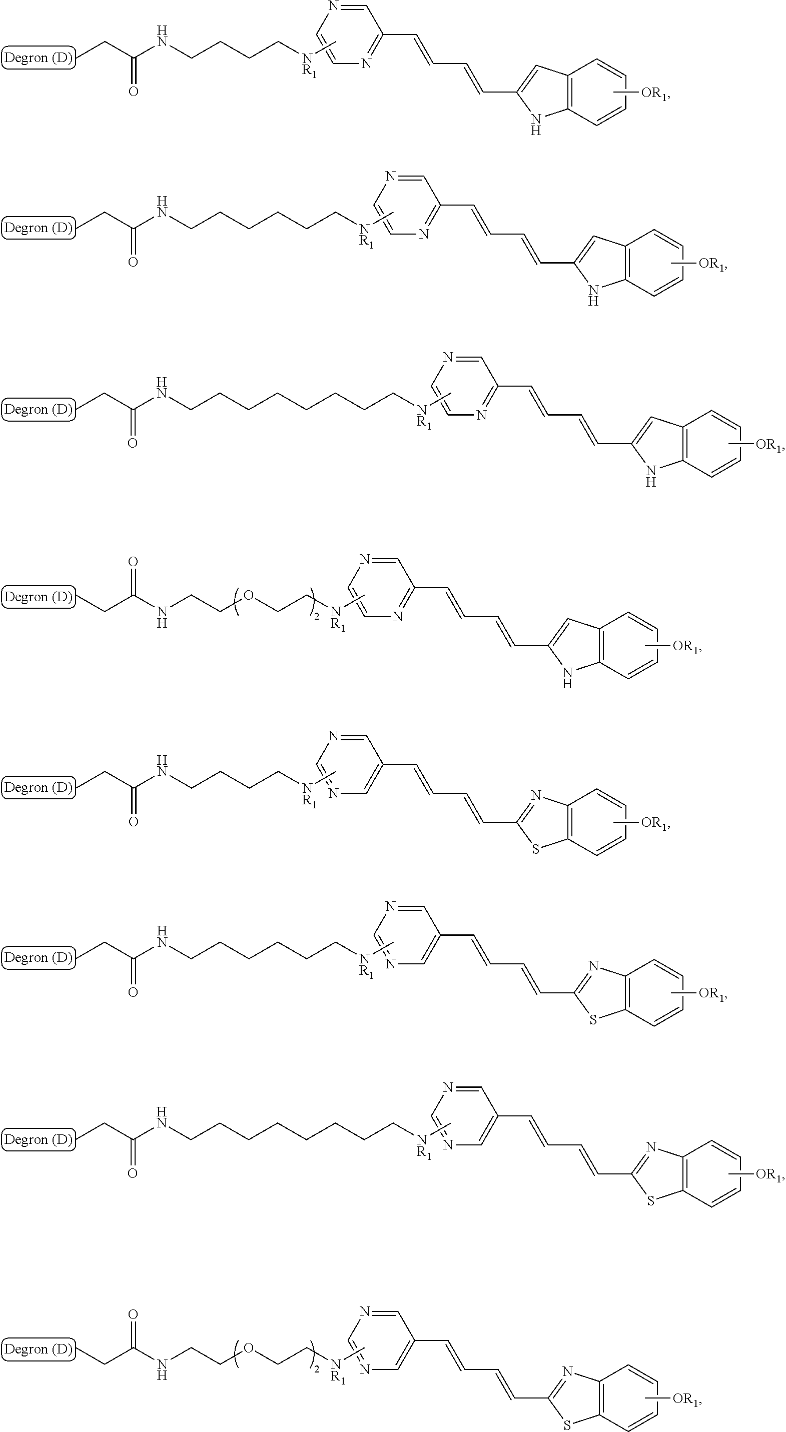

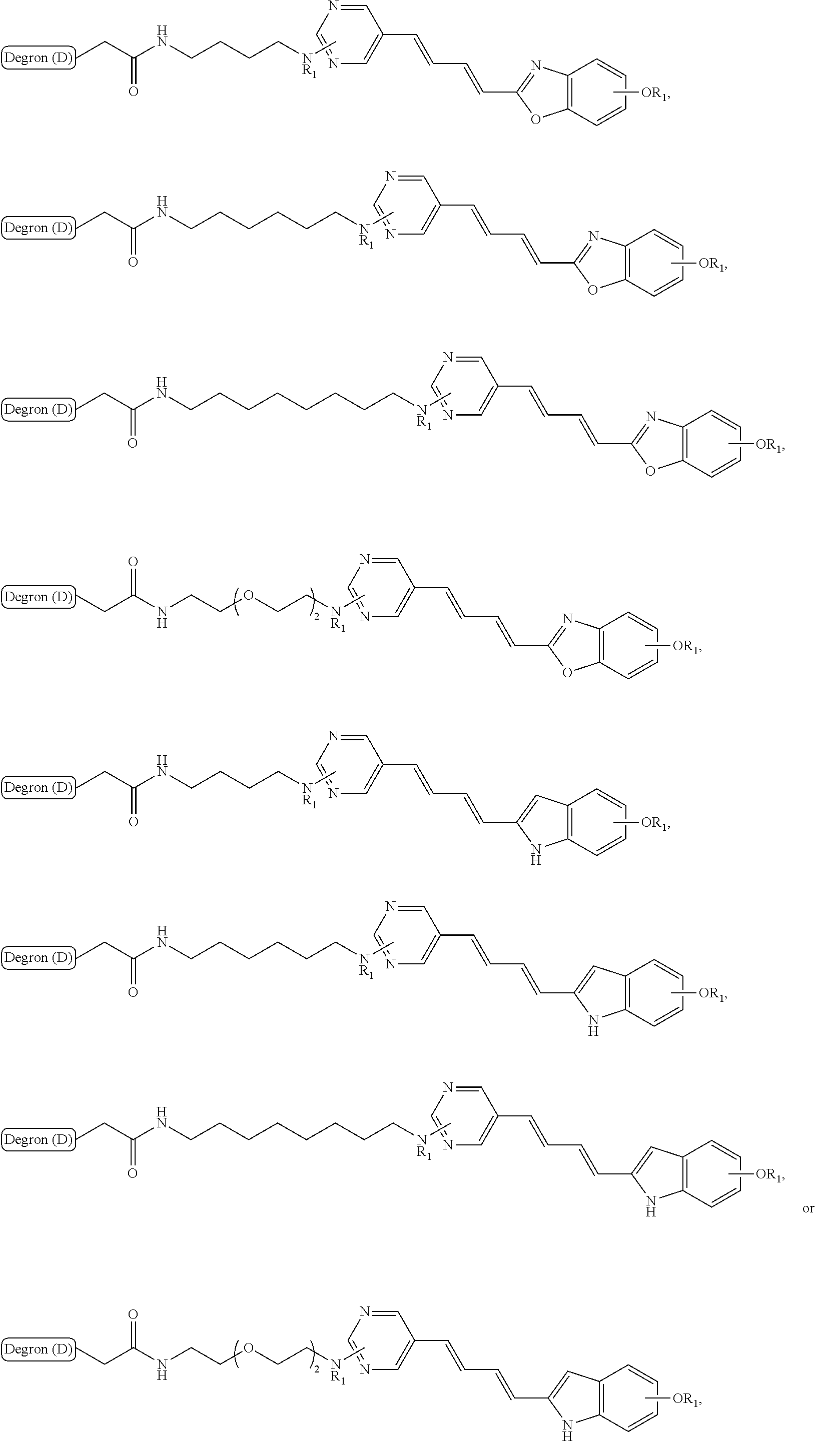

or a pharmaceutically acceptable salt or stereoisomer thereof.

### Degrons

The Ubiquitin-Proteasome Pathway (UPP) is a critical cellular pathway that regulates key regulator proteins and degrades misfolded or abnormal proteins. UPP is central to multiple cellular processes. The covalent attachment of ubiquitin to specific protein substrates is achieved through the action of E3 ubiquitin ligases. These ligases include over 500 different proteins and are categorized into multiple classes defined by the structural element of their E3 functional activity.

In some embodiments, the degron binds the E3 ubiquitin ligase which is cereblon (CRBN), and is represented by any one of the following structures:

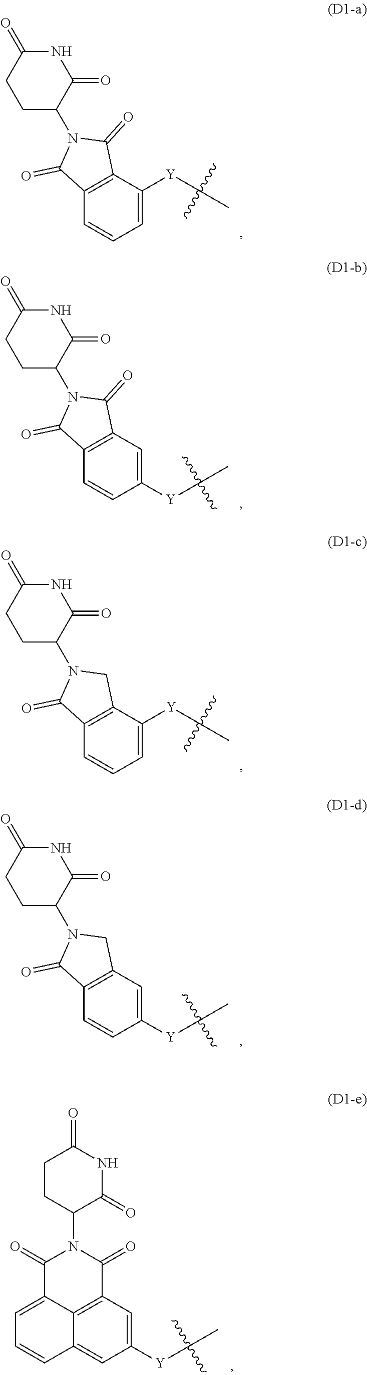

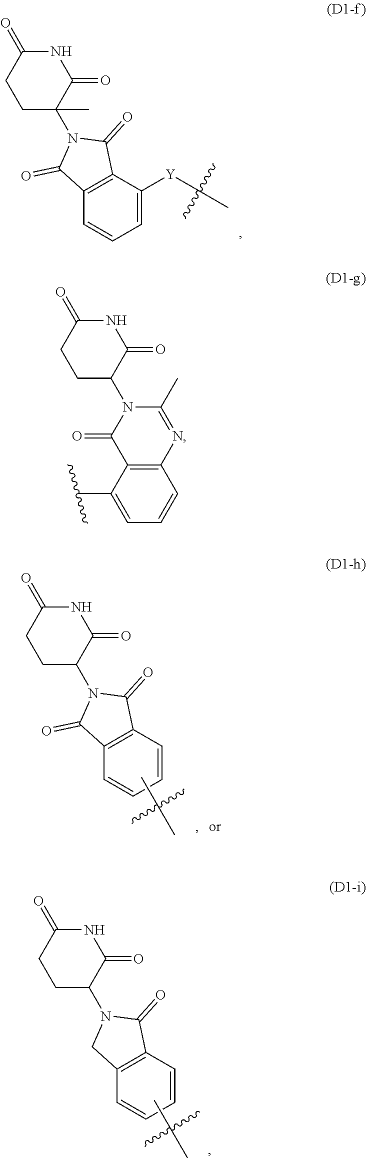

wherein,

### Y is NH or O.

Thus, in some embodiments, the bispecific compounds of the present invention are represented by any of the following structures:

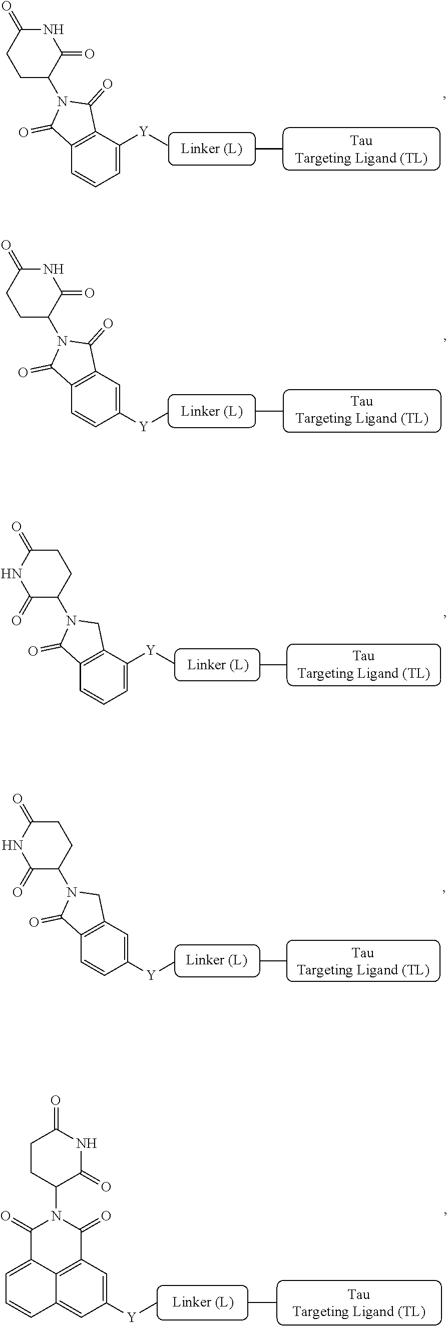

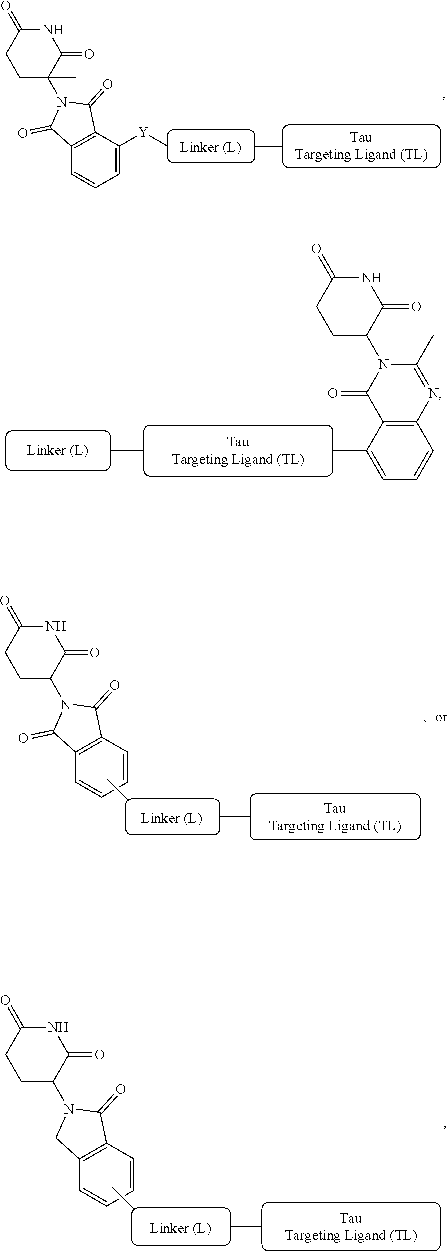

or a pharmaceutically acceptable salt, or stereoisomer thereof.

In some embodiments, the bispecific compounds of the present invention are represented by any one of the following structures:

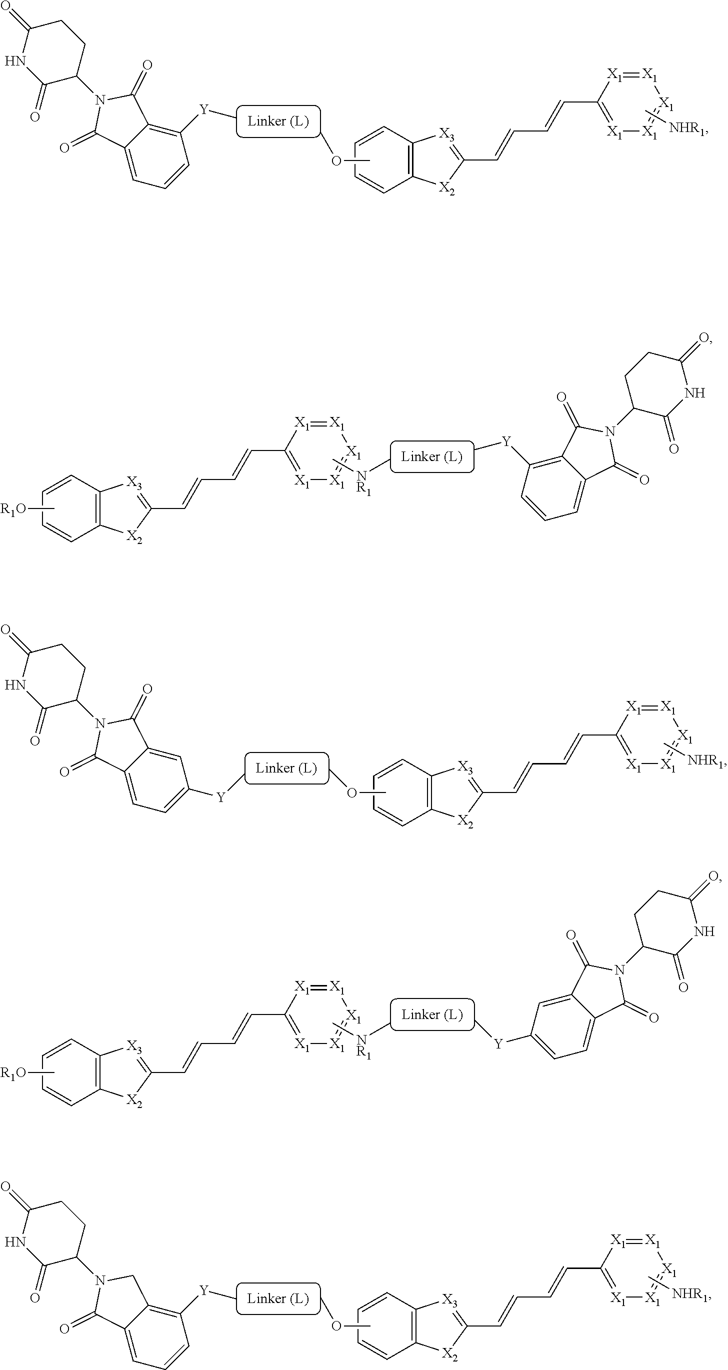

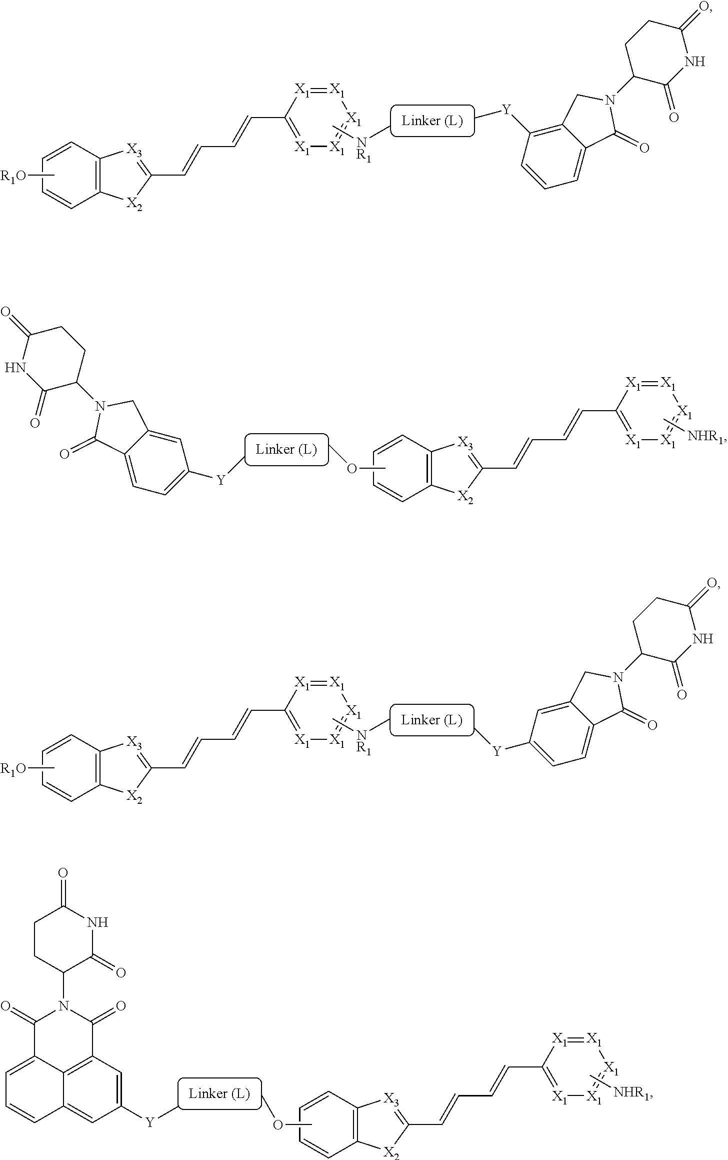

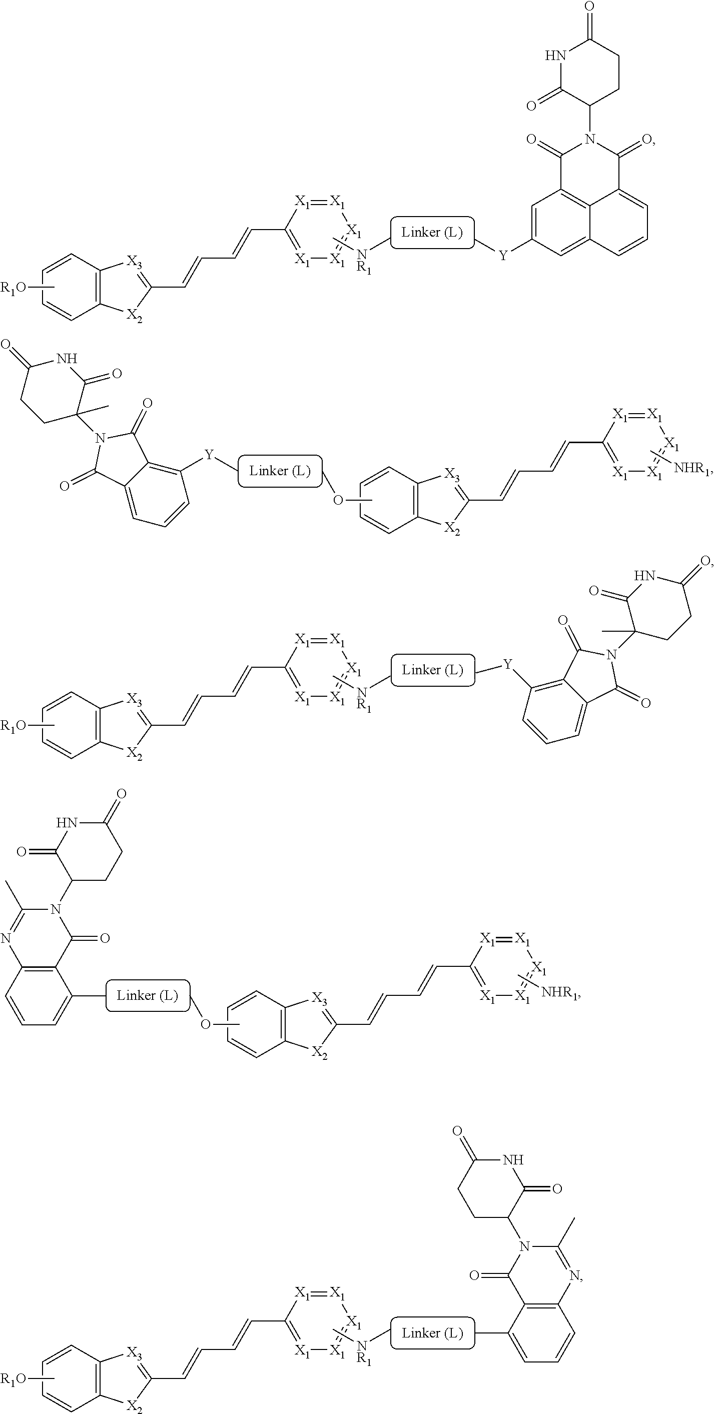

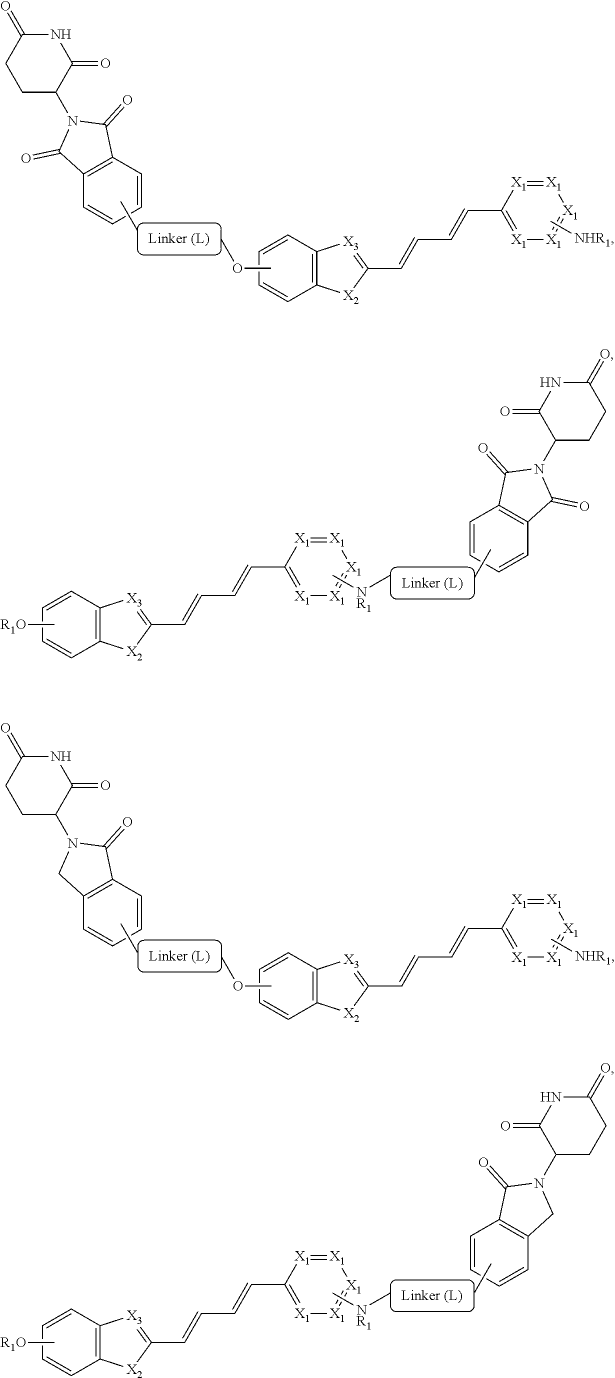

or a pharmaceutically acceptable salt or stereoisomer thereof.

Yet other degrons that bind cereblon and which may be suitable for use in the present invention are disclosed in U.S. Patent Application Publication 2018/0015085 (e.g., the indolinones such as isoindolinones and isoindoline-1,3-diones embraced by formulae IA ad IA′ therein, and the bridged cycloalkyl compounds embraced by formulae IB and IB′ therein).

In some embodiments, the E3 ubiquitin ligase that is bound by the degron is the von Hippel-Lindau (VHL) tumor suppressor. See, Iwai et al., Proc. Nat'l. Acad. Sci. USA 96:12436-41 (1999).

Representative examples of degrons that bind VHL are as follows:

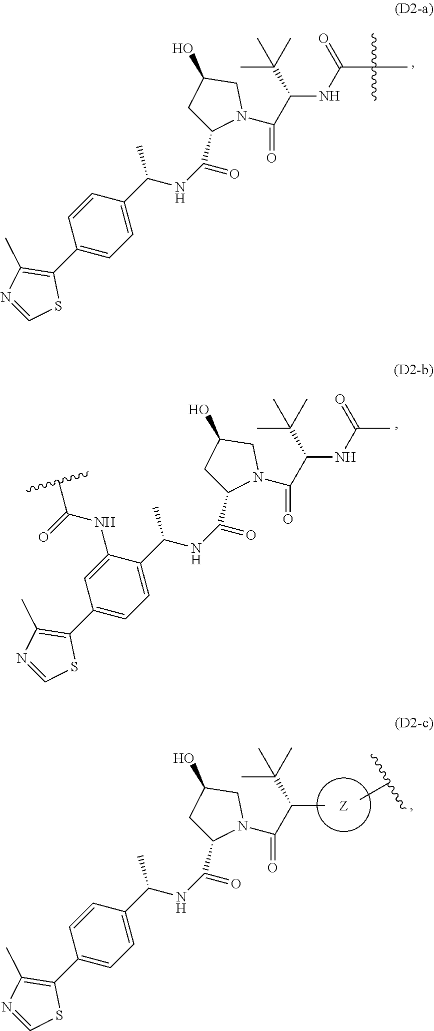

wherein Z is a cyclic group and

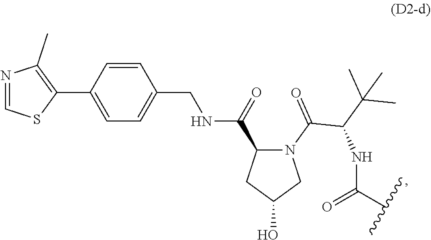 [SMILES: *C(=O)N[C@H](C(=O)N1C[C@H](O)C[C@H]1C(=O)NCc1ccc(-c2scnc2C)cc1)C(C)(C)C]

or a stereoisomer thereof.

In certain embodiments, Z is

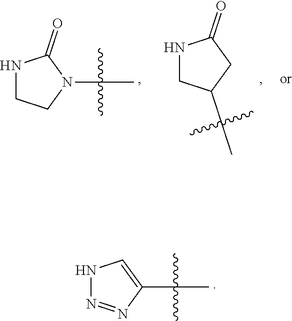 [SMILES: *N1CCNC1=O ; *C1CNC(=O)C1 ; *c1c[nH]nn1]

Thus, in some embodiments, the bispecific compounds of the present invention are represented by any of the following structures:

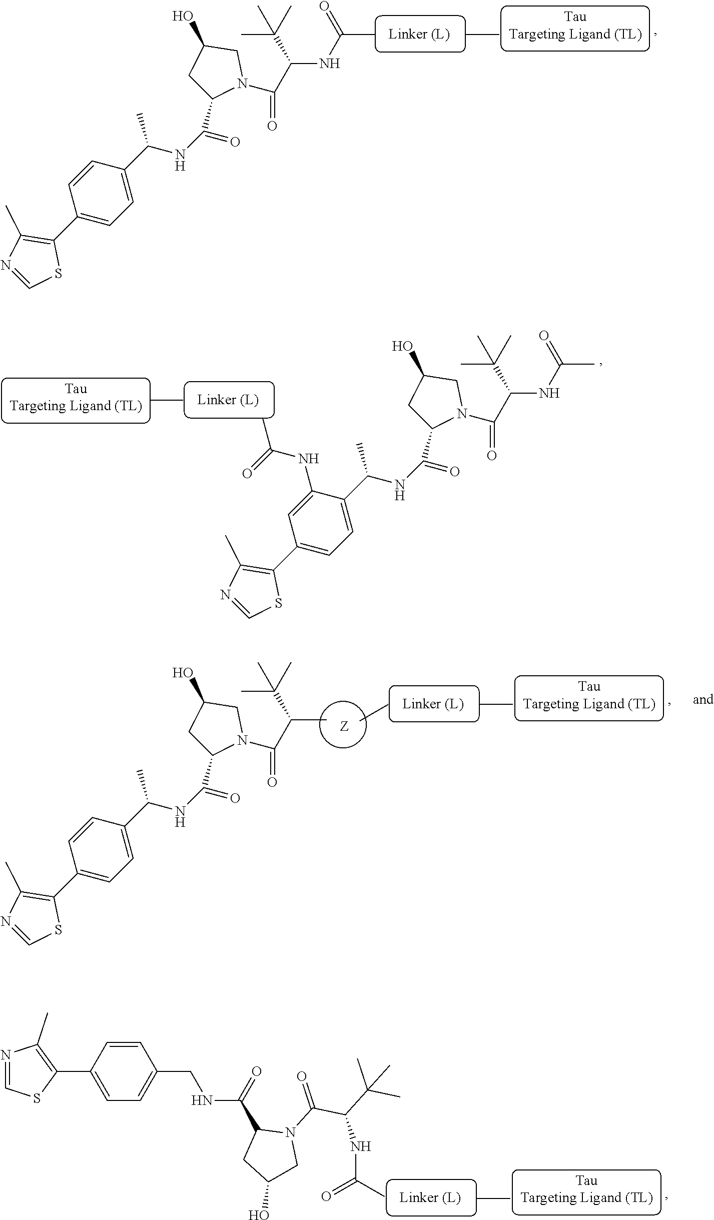

or a pharmaceutically acceptable salt or stereoisomer thereof.

In some embodiments, the bispecific compounds of the present invention are represented by any of the following structures:

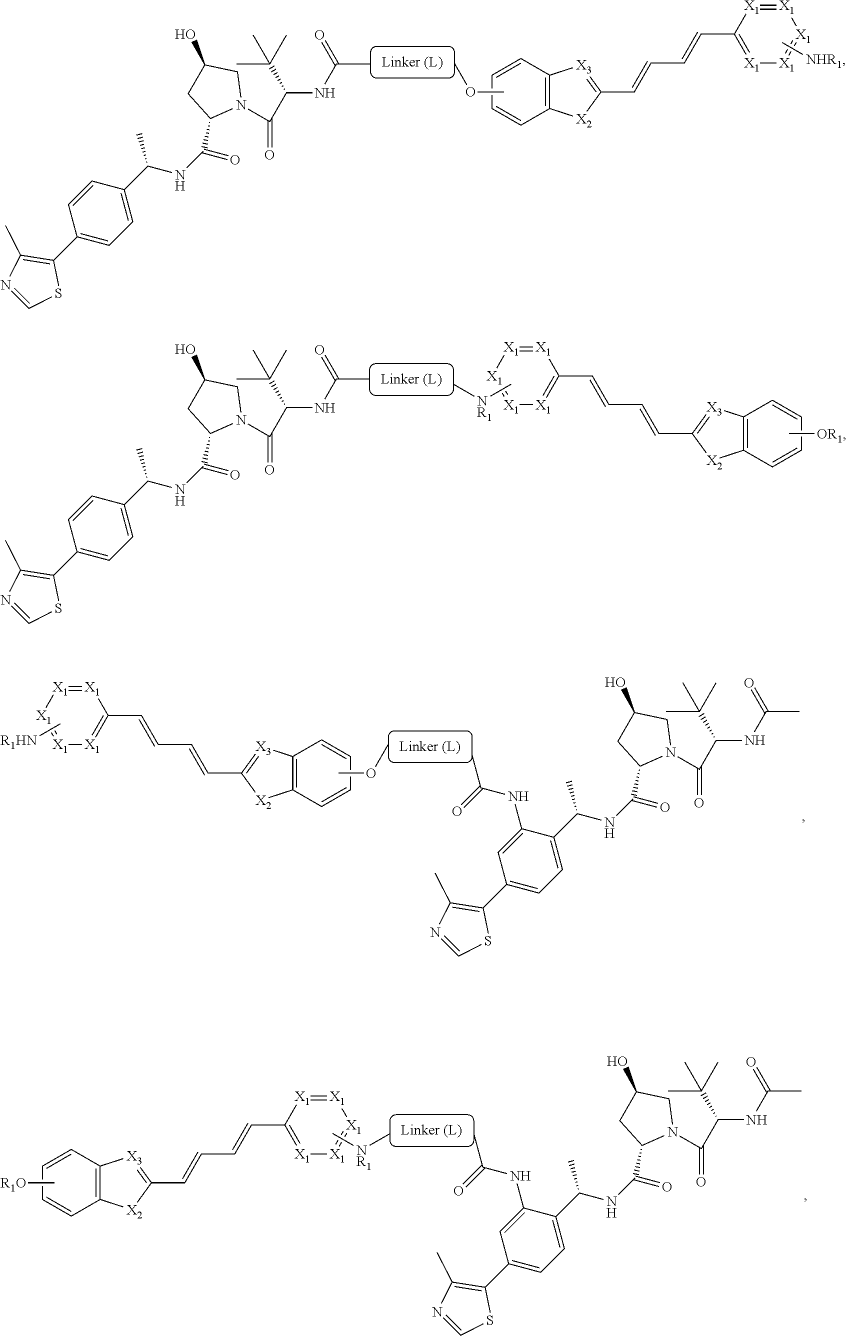

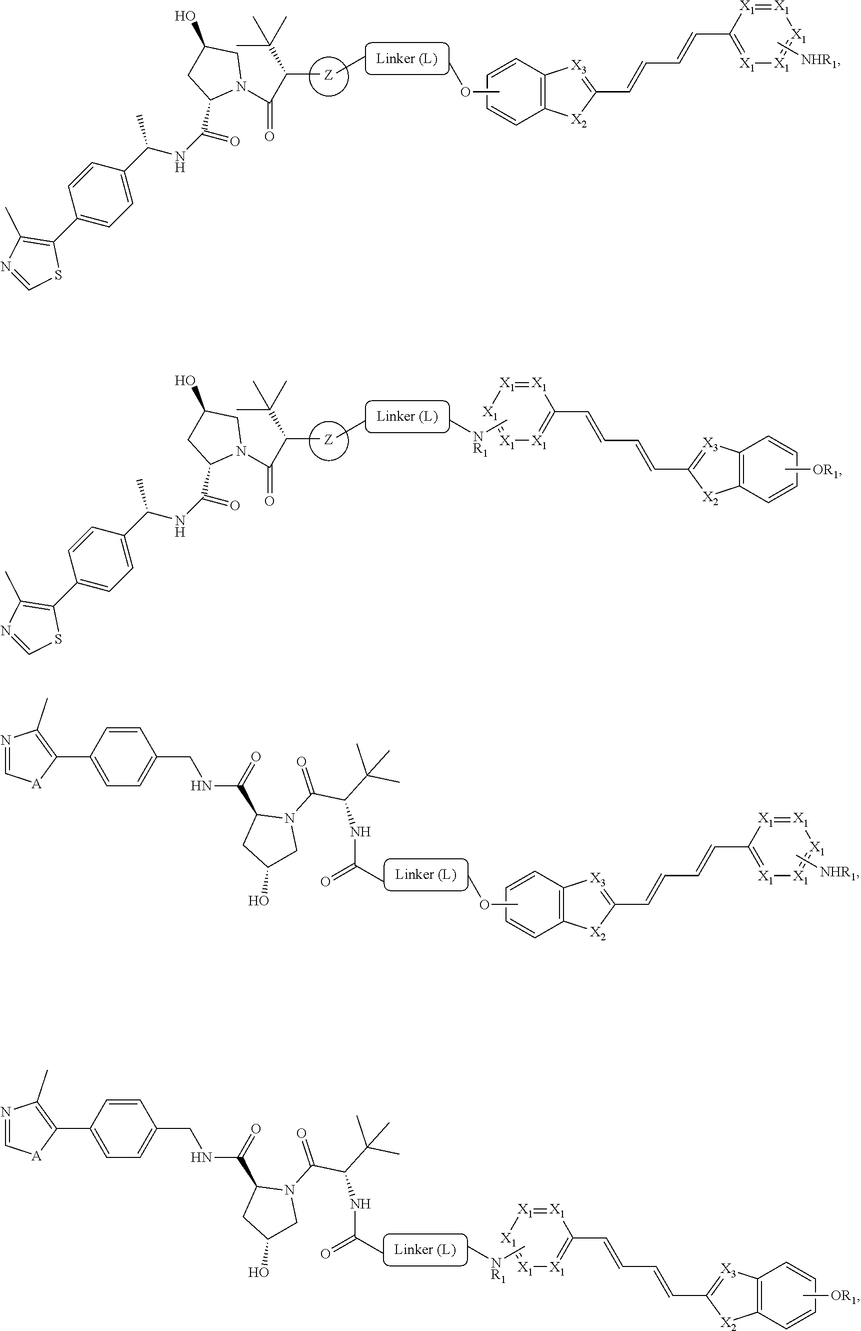

or a pharmaceutically acceptable salt or stereoisomer thereof.

Yet other degrons that bind VHL and which may be suitable for use in the present invention are disclosed in U.S. Patent Application Publication 2017/0121321 A1.

In some embodiments, the E3 ubiquitin ligase that is bound by the degron is an inhibitor of apoptosis protein (IAP). Representative examples of degrons that bind IAP and may be suitable for use in the present invention are represented by any one of the following structures:

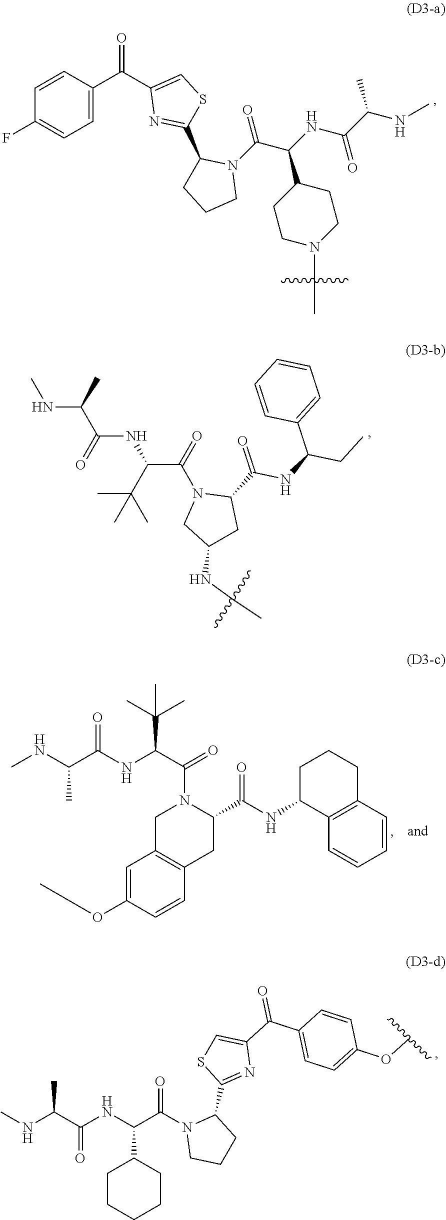

or stereoisomer thereof.

Thus, in some embodiments, the bispecific compounds of the present invention are represented by any one of the following structures:

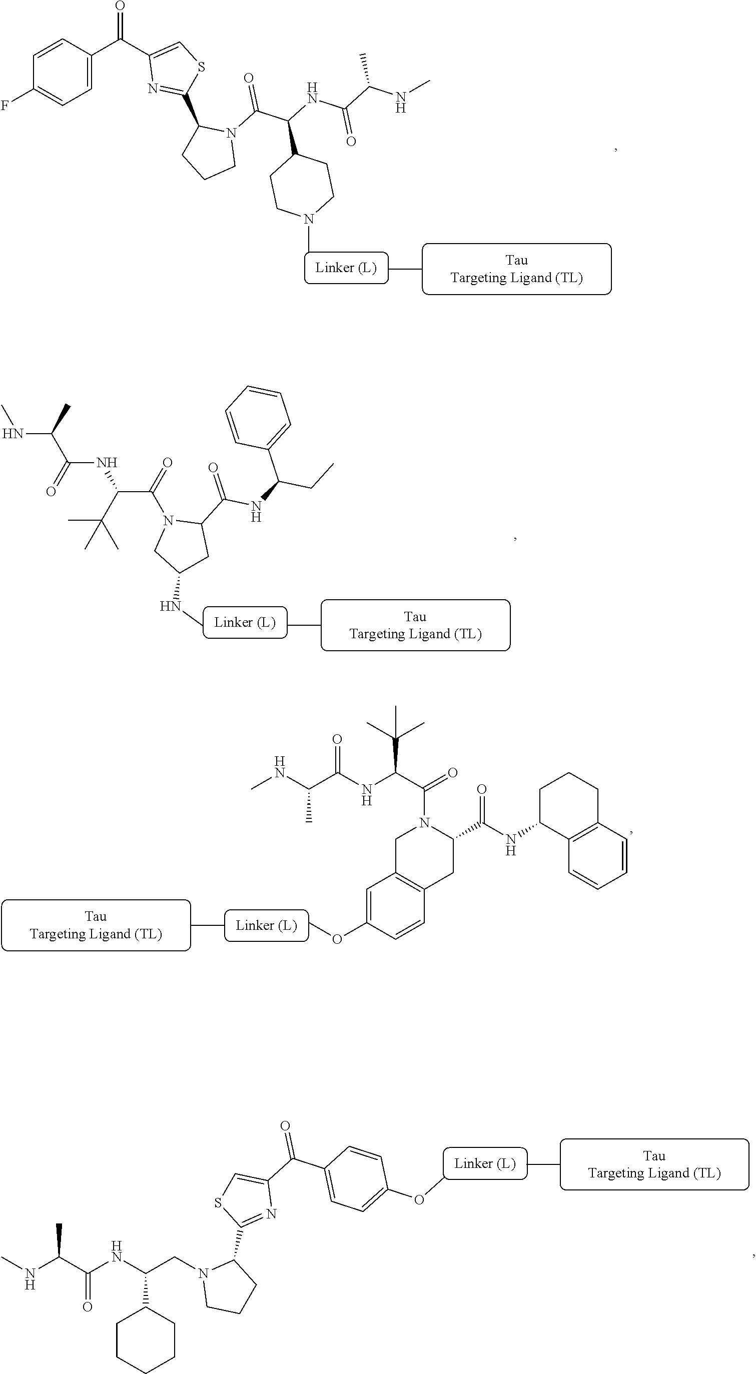

or a pharmaceutically acceptable salt or stereoisomer thereof.

In some embodiments, the bispecific compounds of the present invention are represented by any one of the following structures:

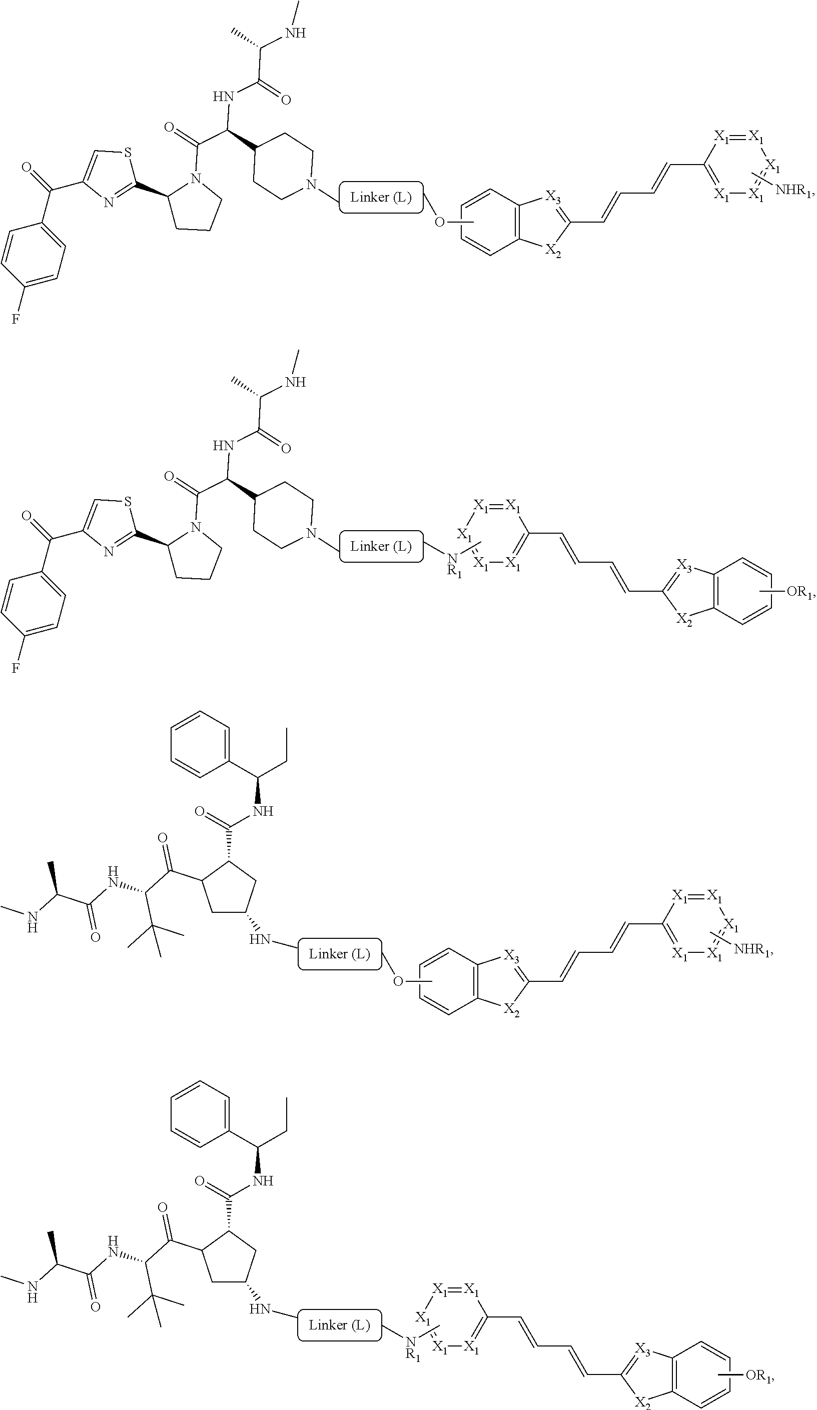

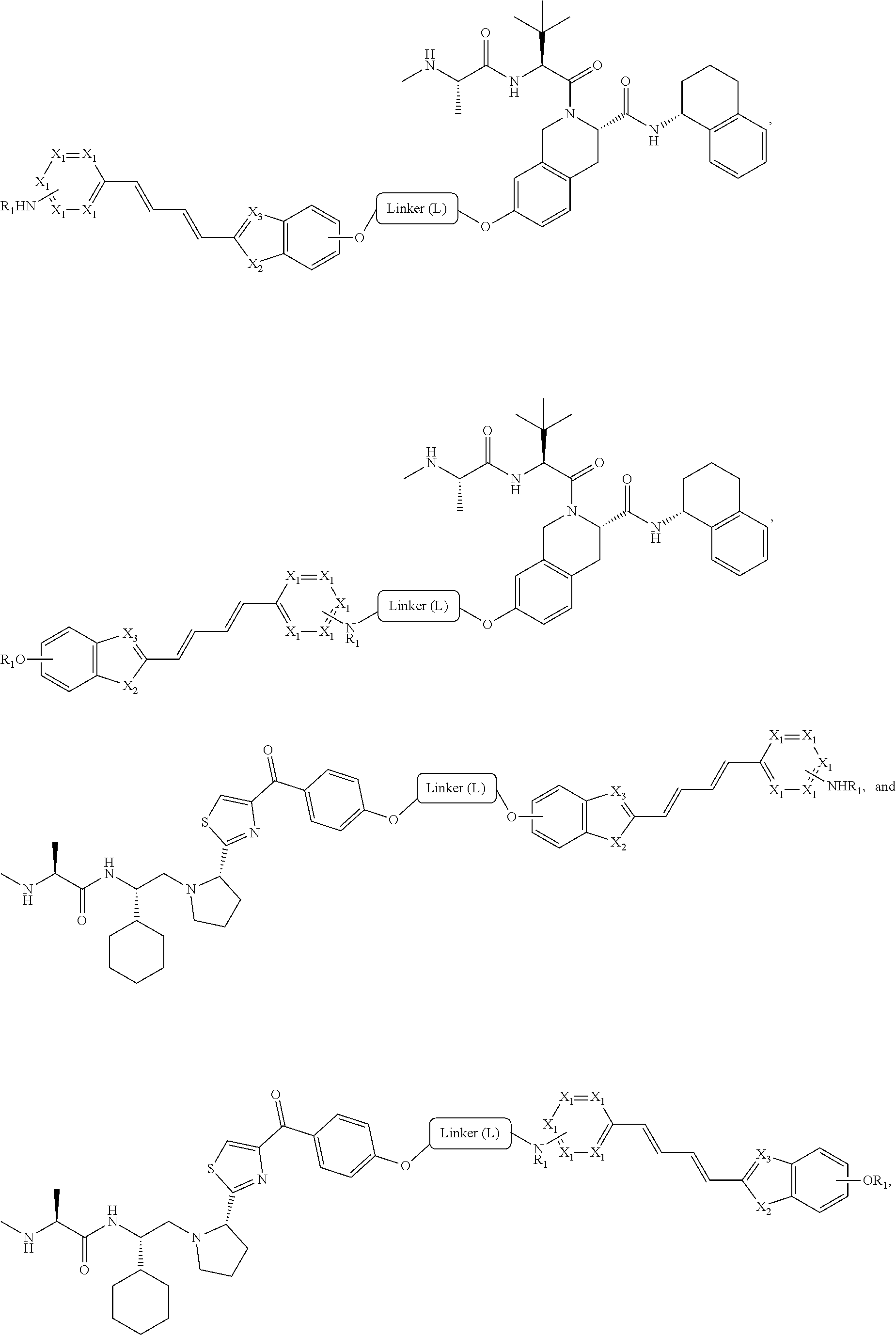

or a pharmaceutically acceptable salt or stereoisomer thereof.

Yet other compounds that bind IAPs and which may be suitable for use as degrons in the present invention are disclosed in International Patent Application Publications WO 2008128171, WO 2008016893, WO 2014060768, WO 2014060767, and WO 2015092420.

In some embodiments, the E3 ubiquitin ligase that is bound by the degron is murine double minute 2 (MDM2). Representative examples of degrons that bind MDM2 and may be suitable for use in the present invention are represented by any one of the following structures:

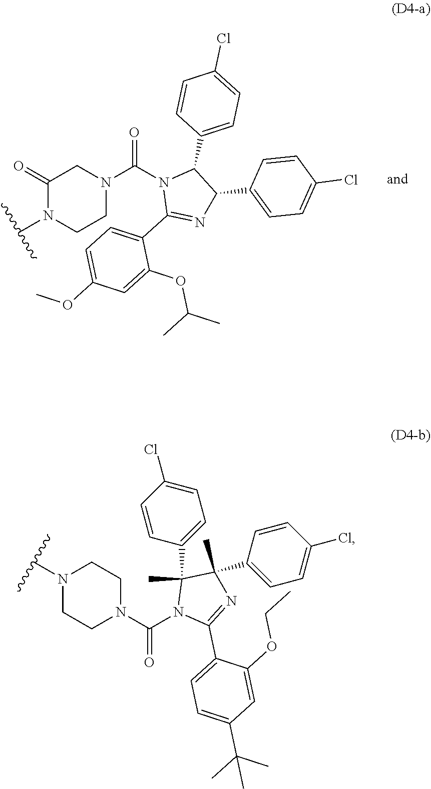 [SMILES: *N1CCN(C(=O)N2C(c3ccc(OC)cc3OC(C)C)=N[C@@H](c3ccc(Cl)cc3)[C@H]2c2ccc(Cl)cc2)CC1=O ; *N1CCN(C(=O)N2C(c3ccc(C(C)(C)C)cc3OCC)=N[C@@](C)(c3ccc(Cl)cc3)[C@@]2(C)c2ccc(Cl)cc2)CC1]

or a stereoisomer thereof.

Thus, in some embodiments, the bispecific compounds of the present invention are represented by any one of the following structures:

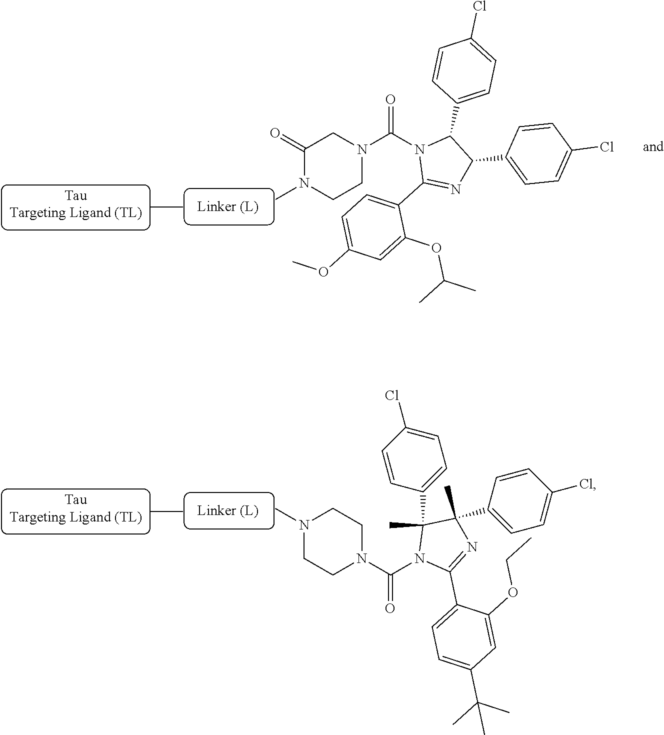

or a pharmaceutically acceptable salt or stereoisomer thereof.

In some embodiments, the bispecific compounds of the present invention are represented by any one of the following structures:

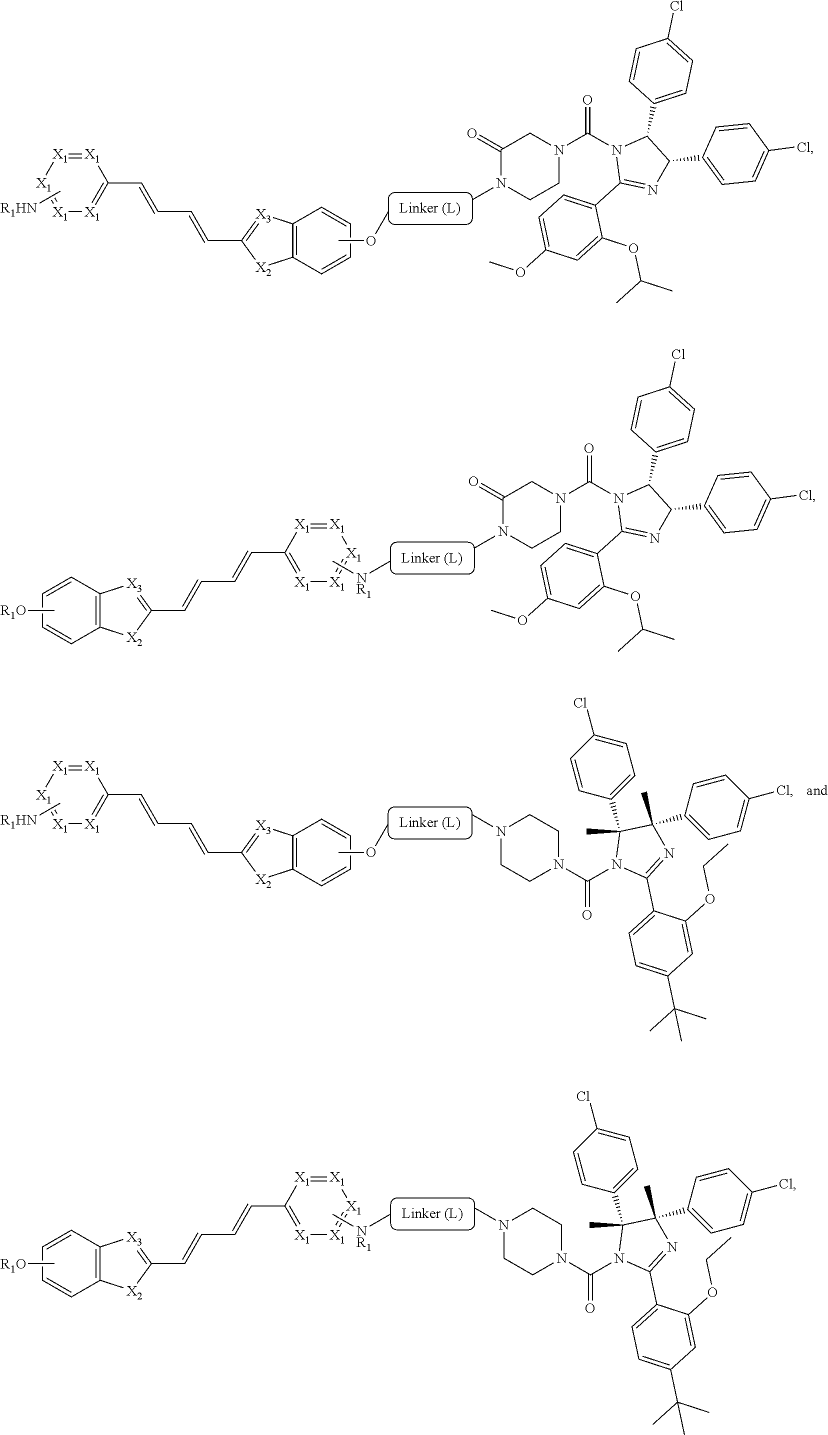

or a pharmaceutically acceptable salt or stereoisomer thereof.

Yet other compounds that bind MDM2 and which may be suitable for use as degrons in the present invention are disclosed in U.S. Pat. No. 9,993,472 B2. MDM2 is known in the art to function as a ubiquitin E3-ligase.

Autophagy is a key homeostatic process that is important for balancing sources of energy at critical times in development and in response to nutrient stress. Autophagy also plays a central role in removing misfolded or aggregated proteins and clearing damaged organelles (Glick et al., J. Pathol., 221(1):3-12 (2010)). Autophagy-mediated clearance serves as a waste disposal system for cells.

In some embodiments, the degron is an autophagy-recruiting tag (i.e. a tag that destines or targets substrates for selective autophagy). Representative autophagy-recruiting tags that may be suitable for use in the present invention are represented by either of the following structures:

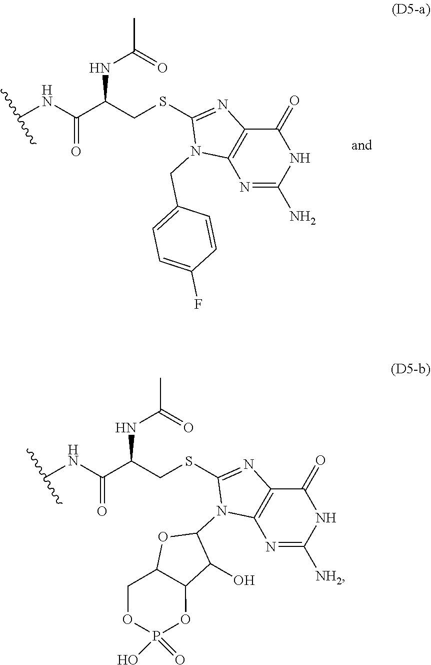 [SMILES: *NC(=O)[C@H](CSc1nc2c(=O)[nH]c(N)nc2n1Cc1ccc(F)cc1)NC(C)=O ; *NC(=O)[C@H](CSc1nc2c(=O)[nH]c(N)nc2n1C1OC2COP(=O)(O)OC2C1O)NC(C)=O]

or stereoisomer thereof.

Thus, in some embodiments, the bispecific compounds of the present invention are represented by any one of the following structures:

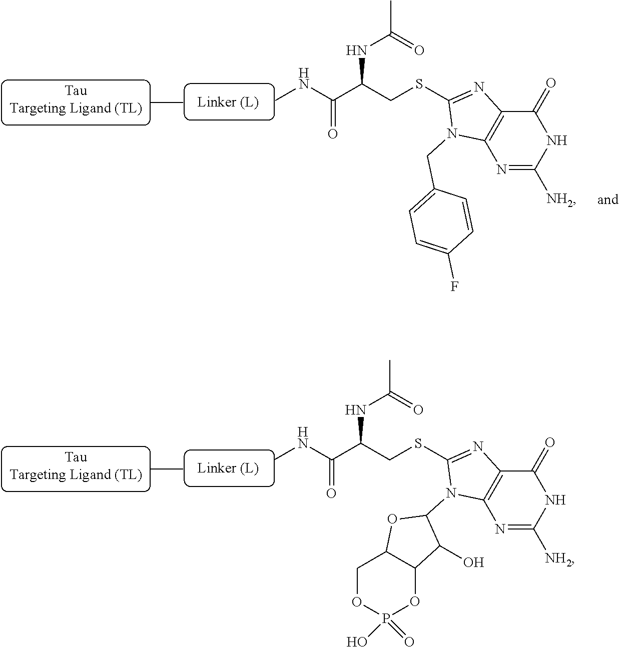

or a pharmaceutically acceptable salt or stereoisomer thereof.

In some embodiments, the bispecific compounds of the present invention are represented by any one of the following structures:

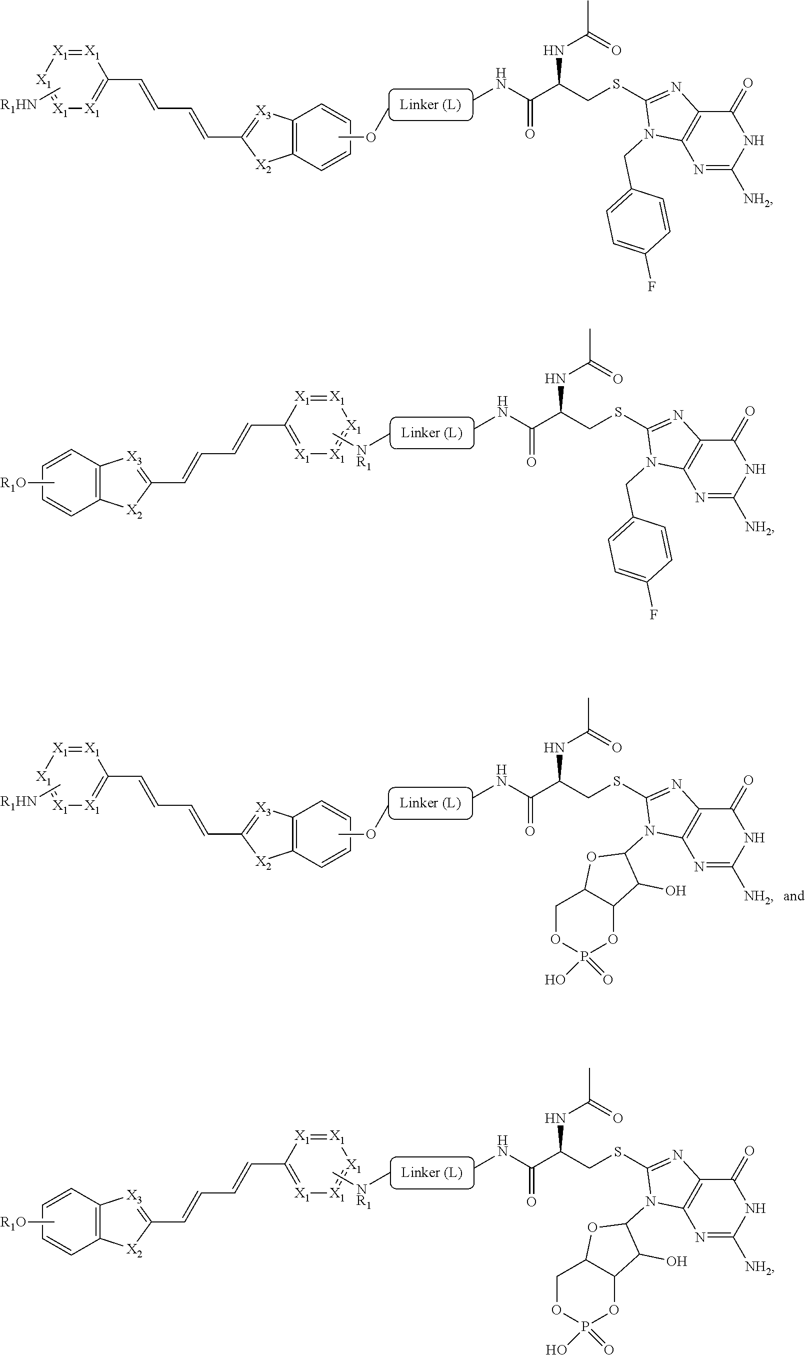

or a pharmaceutically acceptable salt or stereoisomer thereof.

Yet other compounds that can serve as autophagy-recruiting tags and which may be suitable for use as degrons in the present invention are disclosed in United States Patent Application Publication 2019/0290778.

Thus, in some embodiments, the bispecific compounds of this invention are represented by any structures generated by the combination of structures TL1 to TL2, L1 to L10, and the structures of the degrons described herein, including D1 to D5, or a pharmaceutically acceptable salt or stereoisomer thereof.

In some embodiments, the bispecific compounds of the present invention are represented by any one of the following structures:

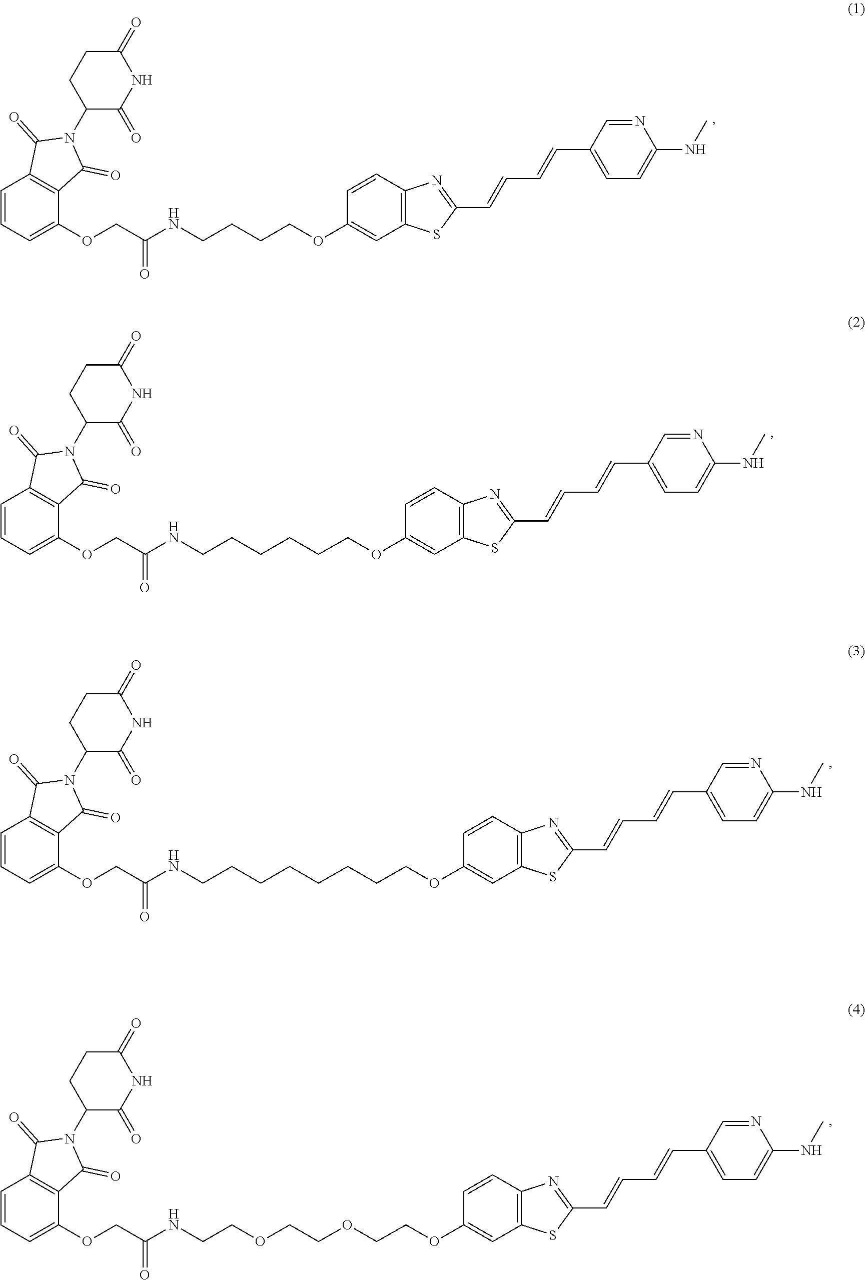 [SMILES: CNc1ccc(/C=C/C=C/c2nc3ccc(OCCCCNC(=O)COc4cccc5c4C(=O)N(C4CCC(=O)NC4=O)C5=O)cc3s2)cn1 ; CNc1ccc(/C=C/C=C/c2nc3ccc(OCCCCCCNC(=O)COc4cccc5c4C(=O)N(C4CCC(=O)NC4=O)C5=O)cc3s2)cn1 ; CNc1ccc(/C=C/C=C/c2nc3ccc(OCCCCCCCCNC(=O)COc4cccc5c4C(=O)N(C4CCC(=O)NC4=O)C5=O)cc3s2)cn1 ; CNc1ccc(/C=C/C=C/c2nc3ccc(OCCOCCOCCNC(=O)COc4cccc5c4C(=O)N(C4CCC(=O)NC4=O)C5=O)cc3s2)cn1]

or a pharmaceutically acceptable salt or stereoisomer thereof.

Bispecific compounds of the present invention may be in the form of a free acid or free base, or a pharmaceutically acceptable salt. As used herein, the term “pharmaceutically acceptable” in the context of a salt refers to a salt of the compound that does not abrogate the biological activity or properties of the compound, and is relatively non-toxic, i.e., the compound in salt form may be administered to a subject without causing undesirable biological effects (such as dizziness or gastric upset) or interacting in a deleterious manner with any of the other components of the composition in which it is contained. The term “pharmaceutically acceptable salt” refers to a product obtained by reaction of the compound of the present invention with a suitable acid or a base. Examples of pharmaceutically acceptable salts of the bispecific compounds of this invention include those derived from suitable inorganic bases such as Li, Na, K, Ca, Mg, Fe, Cu, Al, Zn and Mn salts. Examples of pharmaceutically acceptable, nontoxic acid addition salts are salts of an amino group formed with inorganic acids such as hydrochloride, hydrobromide, hydroiodide, nitrate, sulfate, bisulfate, phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, 4-methylbenzenesulfonate or p-toluenesulfonate salts and the like. Certain bispecific compounds of the invention can form pharmaceutically acceptable salts with various organic bases such as lysine, arginine, guanidine, diethanolamine or metformin. Suitable base salts include aluminum, calcium, lithium, magnesium, potassium, sodium, or zinc salts.

Bispecific compounds of the present invention may have at least one chiral center and thus may be in the form of a stereoisomer, which as used herein, embraces all isomers of individual compounds that differ only in the orientation of their atoms in space. The term stereoisomer includes mirror image isomers (enantiomers which include the (R-) or (S-) configurations of the compounds), mixtures of mirror image isomers (physical mixtures of the enantiomers, and racemates or racemic mixtures) of compounds, geometric (cis/trans or E/Z, R/S) isomers of compounds and isomers of compounds with more than one chiral center that are not mirror images of one another (diastereoisomers). The chiral centers of the compounds may undergo epimerization in vivo; thus, for these compounds, administration of the compound in its (R-) form is considered equivalent to administration of the compound in its (S-) form. Accordingly, the bispecific compounds of the present invention may be made and used in the form of individual isomers and substantially free of other isomers, or in the form of a mixture of various isomers, e.g., racemic mixtures of stereoisomers.

In some embodiments, the bispecific compound is an isotopic derivative in that it has at least one desired isotopic substitution of an atom, at an amount above the natural abundance of the isotope, i.e., enriched. In one embodiment, the compound includes deuterium or multiple deuterium atoms. Substitution with heavier isotopes such as deuterium, i.e. ²H, may afford certain therapeutic advantages resulting from greater metabolic stability, for example, increased in vivo half-life or reduced dosage requirements, and thus may be advantageous in some circumstances.

Thus, the term bispecific compounds of formula (I) embraces the free base form of the bispecific compounds, as well as isotopic derivatives, N-oxides, crystalline forms (also known as polymorphs), active metabolites of the bispecific compounds having the same type of activity, prodrugs, tautomers, and unsolvated as well as solvated (e.g., hydrated) forms with pharmaceutically acceptable solvents such as water, ethanol, and the like, of the bispecific compounds.

### Methods of Synthesis

In another aspect, the present invention is directed to a method for making a bispecific compound of formula (I), or a pharmaceutically acceptable salt or stereoisomer thereof. Broadly, the inventive compounds or pharmaceutically-acceptable salts or stereoisomers thereof may be prepared by any process known to be applicable to the preparation of chemically related compounds. The compounds of the present invention will be better understood in connection with the synthetic schemes that described in various working examples and which illustrate non-limiting methods by which the compounds of the invention may be prepared.

### Pharmaceutical Compositions

Another aspect of the present invention is directed to a pharmaceutical composition that includes a therapeutically effective amount of the bispecific compound of formula (I) or a pharmaceutically acceptable salt or stereoisomer thereof, and a pharmaceutically acceptable carrier. The term “pharmaceutically acceptable carrier,” as known in the art, refers to a pharmaceutically acceptable material, composition or vehicle, suitable for administering compounds of the present invention to mammals. Suitable carriers may include, for example, liquids (both aqueous and non-aqueous alike, and combinations thereof), solids, encapsulating materials, gases, and combinations thereof (e.g., semi-solids), and gases, that function to carry or transport the compound from one organ, or portion of the body, to another organ, or portion of the body. A carrier is “acceptable” in the sense of being physiologically inert to and compatible with the other ingredients of the formulation and not injurious to the subject or patient. Depending on the type of formulation, the composition may include one or more pharmaceutically acceptable excipients.

Broadly, bispecific compounds of formula (I) may be formulated into a given type of composition in accordance with conventional pharmaceutical practice such as conventional mixing, dissolving, granulating, dragee-making, levigating, emulsifying, encapsulating, entrapping and compression processes (see, e.g., Remington: The Science and Practice of Pharmacy (20th ed.), ed. A. R. Gennaro, Lippincott Williams & Wilkins, 2000, and Encyclopedia of Pharmaceutical Technology, eds. J. Swarbrick and J. C. Boylan, 1988-1999, Marcel Dekker, New York). The type of formulation depends on the mode of administration which may include enteral (e.g., oral, buccal, sublingual and rectal), parenteral (e.g., subcutaneous (s.c.), intravenous (i.v.), intramuscular (i.m.), and intrasternal injection, or infusion techniques, intra-ocular, intra-arterial, intramedullary, intrathecal, intraventricular, transdermal, interdermal, intravaginal, intraperitoneal, mucosal, nasal, intratracheal instillation, bronchial instillation, and inhalation) and topical (e.g., transdermal). In general, the most appropriate route of administration will depend upon a variety of factors including, for example, the nature of the agent (e.g., its stability in the environment of the gastrointestinal tract), and/or the condition of the subject (e.g., whether the subject is able to tolerate oral administration). For example, parenteral (e.g., intravenous) administration may also be advantageous in that the compound may be administered relatively quickly such as in the case of a single-dose treatment and/or an acute condition.

In some embodiments, the bispecific compounds are formulated for oral or intravenous administration (e.g., systemic intravenous injection).

Accordingly, bispecific compounds of the present invention may be formulated into solid compositions (e.g., powders, tablets, dispersible granules, capsules, cachets, and suppositories), liquid compositions (e.g., solutions in which the compound is dissolved, suspensions in which solid particles of the compound are dispersed, emulsions, and solutions containing liposomes, micelles, or nanoparticles, syrups and elixirs); semi-solid compositions (e.g., gels, suspensions and creams); and gases (e.g., propellants for aerosol compositions). Compounds may also be formulated for rapid, intermediate or extended release.

Solid dosage forms for oral administration include capsules, tablets, pills, powders, and granules. In such solid dosage forms, the bispecific compound is mixed with a carrier such as sodium citrate or dicalcium phosphate and an additional carrier or excipient such as a) fillers or extenders such as starches, lactose, sucrose, glucose, mannitol, and silicic acid, b) binders such as, for example, methylcellulose, microcrystalline cellulose, hydroxypropyl methylcellulose, carboxymethylcellulose, sodium carboxymethylcellulose, alginates, gelatin, polyvinylpyrrolidinone, sucrose, and acacia, c) humectants such as glycerol, d) disintegrating agents such as crosslinked polymers (e.g., crosslinked polyvinylpyrrolidone (crospovidone), crosslinked sodium carboxymethyl cellulose (croscarmellose sodium), sodium starch glycolate, agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also include buffering agents. Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings. They may further contain an opacifying agent.

In some embodiments, bispecific compounds of the present invention may be formulated in a hard or soft gelatin capsule. Representative excipients that may be used include pregelatinized starch, magnesium stearate, mannitol, sodium stearyl fumarate, lactose anhydrous, microcrystalline cellulose and croscarmellose sodium. Gelatin shells may include gelatin, titanium dioxide, iron oxides and colorants.

Liquid dosage forms for oral administration include solutions, suspensions, emulsions, micro-emulsions, syrups and elixirs. In addition to the bispecific compound, the liquid dosage forms may contain an aqueous or non-aqueous carrier (depending upon the solubility of the compounds) commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Oral compositions may also include an excipients such as wetting agents, suspending agents, coloring, sweetening, flavoring, and perfuming agents.

Injectable preparations may include sterile aqueous solutions or oleaginous suspensions. They may be formulated according to standard techniques using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. The effect of the compound may be prolonged by slowing its absorption, which may be accomplished by the use of a liquid suspension or crystalline or amorphous material with poor water solubility. Prolonged absorption of the compound from a parenterally administered formulation may also be accomplished by suspending the compound in an oily vehicle.

In certain embodiments, bispecific compounds of formula (I) may be administered in a local rather than systemic manner, for example, via injection of the conjugate directly into an organ, often in a depot preparation or sustained release formulation. In specific embodiments, long acting formulations are administered by implantation (for example subcutaneously or intramuscularly) or by intramuscular injection. Injectable depot forms are made by forming microencapsule matrices of the compound in a biodegradable polymer, e.g., polylactide-polyglycolides, poly(orthoesters) and poly(anhydrides). The rate of release of the compound may be controlled by varying the ratio of compound to polymer and the nature of the particular polymer employed. Depot injectable formulations are also prepared by entrapping the compound in liposomes or microemulsions that are compatible with body tissues. Furthermore, in other embodiments, the compound is delivered in a targeted drug delivery system, for example, in a liposome coated with organ-specific antibody. In such embodiments, the liposomes are targeted to and taken up selectively by the organ.

The bispecific compounds may be formulated for buccal or sublingual administration, examples of which include tablets, lozenges and gels.

The bispecific compounds may be formulated for administration by inhalation. Various forms suitable for administration by inhalation include aerosols, mists or powders. Pharmaceutical compositions may be delivered in the form of an aerosol spray presentation from pressurized packs or a nebulizer, with the use of a suitable propellant (e.g., dichlorodifluoromethane, trichlorofluoromethane, dichlorotetrafluoroethane, carbon dioxide or other suitable gas). In some embodiments, the dosage unit of a pressurized aerosol may be determined by providing a valve to deliver a metered amount. In some embodiments, capsules and cartridges including gelatin, for example, for use in an inhaler or insufflator, may be formulated containing a powder mix of the compound and a suitable powder base such as lactose or starch.

Bispecific compounds of formula (I) may be formulated for topical administration which as used herein, refers to administration intradermally by application of the formulation to the epidermis. These types of compositions are typically in the form of ointments, pastes, creams, lotions, gels, solutions and sprays.

Representative examples of carriers useful in formulating bispecific compounds for topical application include solvents (e.g., alcohols, poly alcohols, water), creams, lotions, ointments, oils, plasters, liposomes, powders, emulsions, microemulsions, and buffered solutions (e.g., hypotonic or buffered saline). Creams, for example, may be formulated using saturated or unsaturated fatty acids such as stearic acid, palmitic acid, oleic acid, palmito-oleic acid, cetyl, or oleyl alcohols. Creams may also contain a non-ionic surfactant such as polyoxy-40-stearate.

In some embodiments, the topical formulations may also include an excipient, an example of which is a penetration enhancing agent. These agents are capable of transporting a pharmacologically active compound through the stratum corneum and into the epidermis or dermis, preferably, with little or no systemic absorption. A wide variety of compounds have been evaluated as to their effectiveness in enhancing the rate of penetration of drugs through the skin. See, for example, Percutaneous Penetration Enhancers, Maibach H. I. and Smith H. E. (eds.), CRC Press, Inc., Boca Raton, Fla. (1995), which surveys the use and testing of various skin penetration enhancers, and Buyuktimkin et al., Chemical Means of Transdermal Drug Permeation Enhancement in Transdermal and Topical Drug Delivery Systems, Gosh T. K., Pfister W. R., Yum S. I. (Eds.), Interpharm Press Inc., Buffalo Grove, Ill. (1997). Representative examples of penetration enhancing agents include triglycerides (e.g., soybean oil), aloe compositions (e.g., aloe-vera gel), ethyl alcohol, isopropyl alcohol, octolyphenylpolyethylene glycol, oleic acid, polyethylene glycol 400, propylene glycol, N-decylmethylsulfoxide, fatty acid esters (e.g., isopropyl myristate, methyl laurate, glycerol monooleate, and propylene glycol monooleate), and N-methylpyrrolidone.

Representative examples of yet other excipients that may be included in topical as well as in other types of formulations (to the extent they are compatible), include preservatives, antioxidants, moisturizers, emollients, buffering agents, solubilizing agents, skin protectants, and surfactants. Suitable preservatives include alcohols, quaternary amines, organic acids, parabens, and phenols. Suitable antioxidants include ascorbic acid and its esters, sodium bisulfate, butylated hydroxytoluene, butylated hydroxyanisole, tocopherols, and chelating agents like EDTA and citric acid. Suitable moisturizers include glycerine, sorbitol, polyethylene glycols, urea, and propylene glycol. Suitable buffering agents include citric, hydrochloric, and lactic acid buffers. Suitable solubilizing agents include quaternary ammonium chlorides, cyclodextrins, benzyl benzoate, lecithin, and polysorbates. Suitable skin protectants include vitamin E oil, allatoin, dimethicone, glycerin, petrolatum, and zinc oxide.

Transdermal formulations typically employ transdermal delivery devices and transdermal delivery patches wherein the compound is formulated in lipophilic emulsions or buffered, aqueous solutions, dissolved and/or dispersed in a polymer or an adhesive. Patches may be constructed for continuous, pulsatile, or on demand delivery of pharmaceutical agents. Transdermal delivery of the compounds may be accomplished by means of an iontophoretic patch. Transdermal patches may provide controlled delivery of the compounds wherein the rate of absorption is slowed by using rate-controlling membranes or by trapping the compound within a polymer matrix or gel. Absorption enhancers may be used to increase absorption, examples of which include absorbable pharmaceutically acceptable solvents that assist passage through the skin.

Ophthalmic formulations include eye drops.

Formulations for rectal administration include enemas, rectal gels, rectal foams, rectal aerosols, and retention enemas, which may contain conventional suppository bases such as cocoa butter or other glycerides, as well as synthetic polymers such as polyvinylpyrrolidone, PEG, and the like. Compositions for rectal or vaginal administration may also be formulated as suppositories which can be prepared by mixing the compound with suitable non-irritating carriers and excipients such as cocoa butter, mixtures of fatty acid glycerides, polyethylene glycol, suppository waxes, and combinations thereof, all of which are solid at ambient temperature but liquid at body temperature and therefore melt in the rectum or vaginal cavity and release the compound.

### Dosage Amounts

As used herein, the term, “therapeutically effective amount” refers to an amount of a bispecific compound of formula (I) or a pharmaceutically acceptable salt or a stereoisomer thereof that is effective in producing the desired therapeutic response in a patient suffering from a disease or disorder mediated by aberrant tau protein activity. The term “therapeutically effective amount” thus includes the amount of the bispecific compound or a pharmaceutically acceptable salt or a stereoisomer thereof, that when administered, induces a positive modification in the disease or disorder to be treated, or is sufficient to prevent development or progression of the disease or disorder, or alleviate to some extent, one or more of the symptoms of the disease or disorder being treated in a subject, or which simply kills or inhibits the growth of diseased cells, or reduces the amounts of aberrant tau protein in diseased cells.

The total daily dosage of the bispecific compounds and usage thereof may be decided in accordance with standard medical practice, e.g., by the attending physician using sound medical judgment. The specific therapeutically effective dose for any particular subject will depend upon a variety of factors including the disease or disorder being treated and the severity thereof (e.g., its present status); the activity of the bispecific compound employed; the specific composition employed; the age, body weight, general health, sex and diet of the subject; the time of administration, route of administration, and rate of excretion of the bispecific compound employed; the duration of the treatment; drugs used in combination or coincidental with the specific compound employed; and like factors well known in the medical arts (see, for example, Hardman et al., eds., Goodman and Gilman's The Pharmacological Basis of Therapeutics, 10th Edition, McGraw-Hill Press, 155-173, 2001).

Bispecific compounds of formula (I) and their pharmaceutically acceptable salts and stereoisomers may be effective over a wide dosage range. In some embodiments, the total daily dosage (e.g., for adult humans) may range from about 0.001 to about 1600 mg, from 0.01 to about 1000 mg, from 0.01 to about 500 mg, from about 0.01 to about 100 mg, from about 0.5 to about 100 mg, from 1 to about 100-400 mg per day, from about 1 to about 50 mg per day, from about 5 to about 40 mg per day, and in yet other embodiments from about 10 to about 30 mg per day. Individual dosages may be formulated to contain the desired dosage amount depending upon the number of times the compound is administered per day. By way of example, capsules may be formulated with from about 1 to about 200 mg of compound (e.g., 1, 2, 2.5, 3, 4, 5, 10, 15, 20, 25, 50, 100, 150, and 200 mg). In some embodiments, the compound may be administered at a dose in range from about 0.01 mg to about 200 mg/kg of body weight per day. In some embodiments, a dose of from 0.1 to 100, e.g., from 1 to 30 mg/kg per day in one or more dosages per day may be effective. By way of example, a suitable dose for oral administration may be in the range of 1-30 mg/kg of body weight per day, and a suitable dose for intravenous administration may be in the range of 1-10 mg/kg of body weight per day.

### Methods of Use

The present invention is directed to treating a disease or disorder characterized or mediated by aberrant tau protein activity (e.g., elevated levels of tau or otherwise functionally abnormal or dysfunctional, e.g., deregulated tau levels)(referred to collectively as a “disease or disorder mediated by aberrant tau activity”). A “disease” is generally regarded as a state of health of a subject wherein the subject cannot maintain homeostasis, and wherein if the disease is not ameliorated then the subject's health continues to deteriorate. In contrast, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health.

The term “subject” (or “patient”) as used herein includes all members of the animal kingdom prone to or suffering from the indicated disease or disorder. In some embodiments, the subject is a mammal, e.g., a human or a non-human mammal. The methods are also applicable to companion animals such as dogs and cats as well as livestock such as cows, horses, sheep, goats, pigs, and other domesticated and wild animals. A subject “in need of” the treatment may be suffering from or suspected of suffering from a specific disease or disorder may have been positively diagnosed or otherwise presents with a sufficient number of risk factors or a sufficient number or combination of signs or symptoms such that a medical professional could diagnose or suspect that the subject was suffering from the disease or disorder. Thus, subjects suffering from, and suspected of suffering from, a specific disease or disorder are not necessarily two distinct groups.

In some embodiments, the disease or disorder is neurodegenerative disease or disorder.

Exemplary types of neurodegenerative diseases or disorders that may be amenable to treatment with the bispecific compounds of the present invention includes Parkinson's disease, Prion disease, Huntington's disease, Alzheimer's disease, multiple system atrophy, Pick's disease, progressive supranuclear palsy (PSP), frontotemporal dementia (FTD), corticobasal degeneration (CBD), chronic traumatic encephalopathy, argyrophilic grains disease, tangle-dominant dementia, and primary age-related tauopathy (PART).

In some embodiments, the neurodegenerative disease is Alzheimer's disease.

In some embodiments, the disease or disorder is neuropsychiatric disease or disorder.

Exemplary types of neuropsychiatric diseases or disorders that may be amenable to treatment with the bispecific compounds of the present invention includes autism, schizophrenia, bipolar disorder, an attention deficit disorder, a cognitive deficit disorder, palsy, and depression.

In some embodiments, the neuropsychiatric disease is autism.

In some embodiments, the disease or disorder is neurological disease or disorder.

Exemplary types of neurological diseases or disorders that may be amenable to treatment with the bispecific compounds of the present invention includes infantile tauopathy (e.g. tuberous sclerosis complex hemimegalencephaly, focal cortical dysplasia type 2b, ganglioglioma, or Niemann-Pick disease).

In some embodiments, the disorder is epilepsy or a seizure disorder.

In some embodiments, the disorder is a retinal disorder. In some embodiments, the retinal disorder is glaucoma.

The methods of the present invention may entail administration of bispecific compounds of the invention or pharmaceutical compositions thereof to the patient in a single dose or in multiple doses (e.g., 1, 2, 3, 4, 5, 6, 7, 8, 10, 15, 20, or more doses). For example, the frequency of administration may range from once a day up to about once every eight weeks. In some embodiments, the frequency of administration ranges from about once a day for 1, 2, 3, 4, 5, or 6 weeks, and in other embodiments entails at least one 28-day cycle which includes daily administration for 3 weeks (21 days) and a 7-day “off” period. In other embodiments, the bispecific compound may be dosed twice a day (BID) over the course of two and a half days (for a total of 5 doses) or once a day (QD) over the course of two days (for a total of 2 doses). In other embodiments, the bispecific compound may be dosed once a day (QD) over the course of five days.

### Combination Therapy

The bispecific compounds of formula (I) and their pharmaceutically acceptable salts and stereoisomers may be used in combination or concurrently with at least one other active agent in treating diseases and disorders. The terms “in combination” and “concurrently” in this context mean that the agents are co-administered, which includes substantially contemporaneous administration, by way of the same or separate dosage forms, and by the same or different modes of administration, or sequentially, e.g., as part of the same treatment regimen, or by way of successive treatment regimens. Thus, if given sequentially, at the onset of administration of the second compound, the first of the two compounds is in some cases still detectable at effective concentrations at the site of treatment. The sequence and time interval may be determined such that they can act together (e.g., synergistically to provide an increased benefit than if they were administered otherwise). For example, the therapeutics may be administered at the same time or sequentially in any order at different points in time; however, if not administered at the same time, they may be administered sufficiently close in time so as to provide the desired therapeutic effect, which may be in a synergistic fashion. Thus, the terms are not limited to the administration of the active agents at exactly the same time.

In some embodiments, the treatment regimen may include administration of a bispecific compound of formula (I) or a pharmaceutically acceptable salt or stereoisomer in combination with one or more additional therapeutics known for use in treating the disease or disorder (e.g., neurodegenerative disease). The dosage of the additional therapeutic agent may be the same or even lower than known or recommended doses. See, Hardman et al., eds., Goodman & Gilman's The Pharmacological Basis Of Basis Of Therapeutics, 10th Edition, McGraw-Hill Press, 2001.

In some embodiments, the bifunctional compound of the invention and the additional therapeutic agent may be administered less than 5 minutes apart, less than 30 minutes apart, less than 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. The two or more therapeutic agents may be administered within the same patient visit.

In some embodiments, a bispecific compound of the present invention may be used in combination with one or more of Levodopa, Sinemet, Safinamide, Ropinirole, Pramipexole, Rotigotine Amantadine, Artane, Cogentin, Eldepryl, Zelapar, and Azilect (e.g., for Parkinson's disease. In some embodiments, a bispecific compound of the present invention may be used in combination with one or more of Aricept, Exelon, Razadyne, Namenda, and Namzaric (e.g., for Alzheimer's disease). In some embodiments, a bispecific compound of the present invention may be used in combination with one or more of Xenazine, Haldol, chlorpromazine, Risperdal, Seroquel, Keppra, Klonopin, Celexa, Prozac, Epitol, and Depacon (e.g., for Huntington's disease). In some embodiments, a bispecific compound of the present invention may be used in combination with one or more of trazodone, Zoloft, Luvox, Zyprexa, and Seroquel (e.g., for Pick's syndrome). Representative examples of other active agents known to treat neurodegenerative diseases and disorders, and which may be used in conjunction with the inventive bispecific compounds, include dopaminergic treatments (e.g., Carbidopa-levodopa, pramipexole (Mirapex), ropinirole (Requip) and rotigotine (Neupro, given as a patch)). Apomorphine and monoamine oxidase B (MAO-B) inhibitors (e.g., selegiline (Eldepryl, Zelapar), rasagiline (Azilect) and safinamide (Xadago)) for PD and movement disorders, cholinesterase inhibitors for cognitive disorders (e.g., benztropine (Cogentin) or trihexyphenidyl), antipsychotic drugs for behavioral and psychological symptoms of dementia, as well as agents aimed to slow the development of diseases, such as Riluzole for ALS, cerebellar ataxia and Huntington's disease, non-steroidal anti-inflammatory drugs for Alzheimer's disease, and caffeine A2A receptor antagonists and CERE-120 (adeno-associated virus serotype 2-neurturin) for the neuroprotection of Parkinson's disease.

### Pharmaceutical Kits

The present compositions may be assembled into kits or pharmaceutical systems. Kits or pharmaceutical systems according to this aspect of the invention include a carrier or package such as a box, carton, tube or the like, having in close confinement therein one or more containers, such as vials, tubes, ampoules, or bottles, which contain the compound of the present invention or a pharmaceutical composition. The kits or pharmaceutical systems of the invention may also include printed instructions for using the bispecific compounds and compositions.

These and other aspects of the present invention will be further appreciated upon consideration of the following Examples, which are intended to illustrate certain particular embodiments of the invention but are not intended to limit its scope, as defined by the claims.

### EXAMPLES

### General

¹H and ¹³C NMR spectra were recorded on a Bruker AV-III-400 or 500 MHz NMR spectrometer. Chemical shifts are reported in 6 values in ppm downfield from TMS as the internal standard. ¹H NMR data are reported as follows: chemical shift, multiplicity (s=singlet, d=doublet, t=triplet, q=quartet, b=broad, m=multiplet, quint=quintet), coupling constant (Hz), integration. ¹³C chemical shifts are reported in 6 values in ppm downfield from TMS as the internal standard. Low resolution mass spectra were obtained on Waters Acquity™ UltraPerformance LC® with electrospray ionization and SQ detector by injecting sample in a steady flow of 1 mM ammonium acetate in 20% water-acetonitrile at the rate of 0.2 mL/min. The purity of compounds were determined by analytical HPLC, performed on a Shimadzu Prominence-HPLC with ELSD PDA multi, and a Hypersil BDS C-18 column (250×4.6 mm, 5p) with mobile phase (A) Acetonitrile and mobile phase (B) 5 mM ammonium acetate in water using following gradient of B/A (0 min, 80%), (25 min, 30%), (30 min, 10%), (31 min, 80%), and (36 min, 80%) at 1.0 mL/min flow rate. Analytical thin layer chromatography was performed on 250 μM silica gel F₂₅₄ plates. Preparative thin layer chromatography was performed on 1000 μM silica gel F₂₅₄ plates. Flash column chromatography was performed employing 230-400 mesh silica gel.

### Example 1: Synthesis of 2-((1E,3E)-4-(6-(Methylamino)pyridin-3-yl)buta-1,3-dienyl)benzo[d]thiazol-6-ol

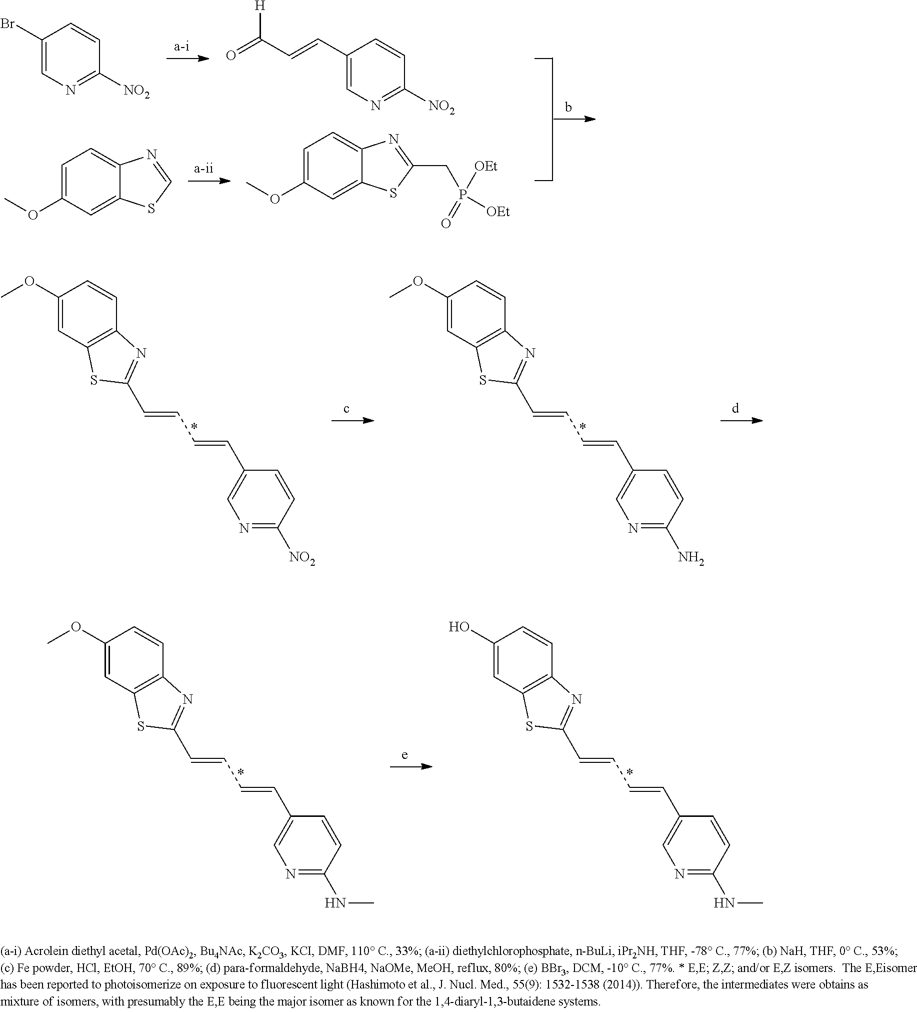

### (E)-3-(6-Nitropyridin-3-yl)acrylaldehyde

A suspension of 5-bromo-2-nitropyridine (2.01 g, 10.0 mmol), acrolein diethyl acetal (4.01 g, 30.0 mmol), K₂CO₃ (2.03 g, 15.0 mmol), tert-butyl acetoacetate (TBAA) (6.01 g, 20.0 mmol), KCl (740 mg, 10 mmol), palladium (II) acetate (224 mg, 1.0 mmol), and dimethylformamide (DMF) (25 mL) under N₂ atmosphere was stirred at 110° C. for 22 hours. The reaction mixture was cooled, diluted with 2 N HCl solution (30 mL) and stirred for 30 minutes. The solution was filtered through a pad of Celite® and washed with ethyl acetate (EtOAc). The combined filtrate was extracted with EtOAc (2×40 mL). The combined organic layers were washed with saturated NaHCO₃ solution (2×20 mL), dried over anhydrous Na₂SO₄, filtered and concentrated to obtain 1.7 g brown solid. The crude product was purified by silica gel column chromatography (0-2% MeOH in CHCl₃) to afford the title compound as a pale yellow solid (0.67 g, 32%). ¹H NMR (400 MHz, CDCl₃) δ 9.83 (d, J=7.6 Hz, 1H), 8.81 (d, J=2.4 Hz, 1H), 8.36 (d, J=8.4 Hz, 1H), 8.22 (dd, J=2.0, 8.4 Hz, 1H), 7.58 (d, J=16.0 Hz, 1H), 6.90 (dd, J=7.2, 16.0, Hz, 1H).

### Diethyl ((6-methoxybenzo[d]thiazol-2-yl)methyl)phosphonate

To a stirred solution of n-BuLi (2.5 M in hexanes, 25 mL, 62.5 mmol) in anhydrous THF (25 mL) was added diisopropylamine (6.55 g, 65.5 mmol) at −78° C. under N₂ atmosphere. The mixture stirred at this temperature for 50 minutes. A solution of 6-methoxy-2-methylbenzothiazole (4.7 g, 26.1 mmol) in anhydrous THF (40 mL) was added dropwise to the mixture and the resulting reddish solution stirred for 30 minutes at −78° C. A solution of diethylchlorophosphate (5.16 g, 30.0 mmol) in anhydrous THF (20 mL) was then added dropwise and the reaction mixture stirred for 10 minutes at −78° C. The reaction mixture was then allowed to warm up to room temperature (rt) and stirred at rt for 1 hour. The reaction was quenched with aqueous 1 N NH₄Cl (100 mL), and extracted with CHCl₃. The combined organic layers were washed with aqueous 2% Na₂CO₃, brine and dried over anhydrous Na₂SO₄, filtered and concentrated in a vacuum. The crude product was purified by column chromatography (0-100% EtOAc in hexanes) to afford the title compound as a red oil (7.0 g, 77%). ¹H NMR (CDCl₃) δ 7.88 (d, J=8.8 Hz, 1H), 7.30 (d, J=2.4 Hz, 1H), 7.07 (dd, J=2.4, 8.8 Hz, 1H), 4.15 (quint, J=7.2 Hz, 4H), 3.87 (s, 3H), 3.69 (d, J=21.6 Hz, 2H), 1.31 (t, J=7.2 Hz, 6H).

### 6-Methoxy-2-((1E,3E)-4-(6-nitropyridin-3-yl)buta-1,3-dienyl)benzo[d]thiazole

To a stirred solution of diethyl ((6-methoxybenzo[d]thiazol yl)methyl)phosphonate (1.93 g, 6.1 mmol) in anhydrous THF (40 mL) was added NaH (60% dispersion in mineral oil, 460 mg, 11.5 mmol) at 0° C. under argon atmosphere. After the mixture stirred for 30 minutes, (E)-3-(6-nitropyridin-3-yl)acrylaldehyde (1.01 g, 5.6 mmol) was added in portions over 5 minutes. The reaction mixture was allowed to warm to rt while stirring overnight. The suspension was concentrated, re-suspended in MeOH, and filtered. The filter cake was washed with MeOH, dried under reduced pressure to obtain the title compound as an orange solid (1.05 g, 53%). ¹H NMR (400 MHz, DMSO-d₆) δ 8.82 (d, J=2.0 Hz, 1H), 8.41-8.31 (m, 2H), 7.88 (d, J=8.8 Hz, 1H), 7.69 (d, J=2.4 Hz, 1H), 7.64-7.54 (m, 1H), 7.46-7.36 (m, 1H), 7.18 (d, J=15.2 Hz, 2H), 7.12 (dd, J=2.8, 8.8 Hz, 1H), 3.85 (s, 3H).

### 5-((1E,3E)-4-(6-Methoxybenzo[d]thiazol-2-yl)buta-1,3-dienyl)pyridin-2-amine

To a stirred solution of 6-methoxy-2-((1E,3E)-4-(6-nitropyridin-3-yl)buta-1,3-dienyl)benzo[d]thiazole (610 mg, 1.88 mmol) in EtOH (50 mL) was added iron powder (500 mg) and 1.0 M HCl solution in water (3 mL) and stirred at 70° C. for 4 hours. The reaction mixture was filtered through a pad of Celite®, and the Celite® pad was washed with 10% MeOH in CHCl₃ (50 mL). The combined organic layers were concentrated and purified by silica gel column chromatography (0-15% MeOH in CHCl₃ with 0.5% NH₄OH as additive) to obtain the title compound as a yellow solid (500 mg, 89%). ¹H NMR (400 MHz, DMSO-d₆) δ 8.08 (d, J=2.4 Hz, 1H), 7.84 (d, J=9.2 Hz, 1H), 7.66 (dd, J=2.4, 8.4 Hz, 1H), 7.29 (d, J 2.4 Hz, 1H), 7.20 (dd, J=10.0, 15.2 Hz, 1H), 7.05 (dd, J=2.8, 9.2 Hz, 1H), 6.85 (d, J=9.6 Hz, 1H), 6.82-6.68 (m, 2H), 6.52 (d, J=8.4 Hz, 1H), 4.88 (bs, 2H), 3.88 (s, 3H).

### 5-((1E,3E)-4-(6-Methoxybenzo[d]thiazol-2-yl)buta-1,3-dienyl)-N-methylpyridin-2-amine

To a stirred solution of 5-((1E,3E)-4-(6-methoxybenzo[d]thiazol-2-yl)buta-1,3-dienyl)pyridin-2-amine (470 mg, 1.52 mmol) and paraformaldehyde (180 mg, 6.0 mmol) in anhydrous MeOH (25 mL) was added NaOMe (810 mg, 15.0 mmol). After the reaction mixture was heated and refluxed for 2 hours, it was cooled to 0° C. and NaBH₄ (240 mg, 6.0 mmol) was added. After 20 minutes, the mixture was heated and refluxed for 2 hours. The reaction was quenched with cold water (25 mL) and extracted with CHCl₃ (2×50 mL). The organic layers were combined and dried over anhydrous Na₂SO₄, filtered and concentrated in a vacuum. The crude product was purified by silica gel column chromatography (0-3% MeOH in CHCl₃) to obtain the title compound as a yellow solid (370 mg, 80%). ¹H NMR (500 MHz, DMSO-d₆) δ 8.10 (d, J=2.0 Hz, 1H), 7.80 (d, J=9.0 Hz, 1H), 7.70 (dd, J=2.5, 9.0 Hz, 1H), 7.62 (d, J=2.5 Hz, 1H), 7.28 (dd, J=10.0, 15.5 Hz, 1H), 7.07 (dd, J=3.0, 9.0 Hz, 1H), 6.98-6.84 (m, 4H), 6.49 (d, J=9.0 Hz, 1H), 3.83 (s, 3H), 2.81 (d, J=5.5 Hz, 3H).

### 2-((1E,3E)-4-(6-(Methylamino)pyridin-3-yl)buta-1,3-dienyl)benzo[d]thiazol-6-ol

To a cooled (−10° C.) suspension of 5-((1E,3E)-4-(6-methoxybenzo[d]thiazol-2-yl)buta-1,3-dienyl)-N-methylpyridin-2-amine (1.05 g, 3.1 mmol) in dry CH₂Cl₂ (10 mL) was added 1 M BBr₃ solution in CH₂Cl₂ (20 mL, 20 mmol) and stirred at −10° C. for 1 hour and then allowed to warm up to rt over 18 hours. The reaction mixture was cooled in an ice-bath, quenched with cold water (10 mL) and stirred for 20 minutes. The resultant heterogeneous mixture was basified with saturated NaHCO₃ solution and the precipitate was filtered. The filter-cake was washed with water and dried under reduced pressure to obtain the title compound as a yellow solid (760 mg, 77%). ¹H NMR (400 MHz, DMSO-d₆) δ 9.83 (s, 1H), 8.09 (d, J=2.0 Hz, 1H), 7.73-7.67 (m, 2H), 7.32 (d, J=2.4 Hz, 1H), 7.22 (dd, J=10.0, 15.2 Hz, 1H), 7.01-6.81 (m, 5H), 6.50 (d, J=8.8 Hz, 1H), 2.81 (d. J=4.8 Hz, 3H). MS (ESI⁺) m/z 310.1 (M+H)⁺.

### Example 2: Synthesis of 2-(2-(2,6-dioxopiperidin-3-yl)-1,3-dioxoisoindolin-4-yloxy)acetic acid

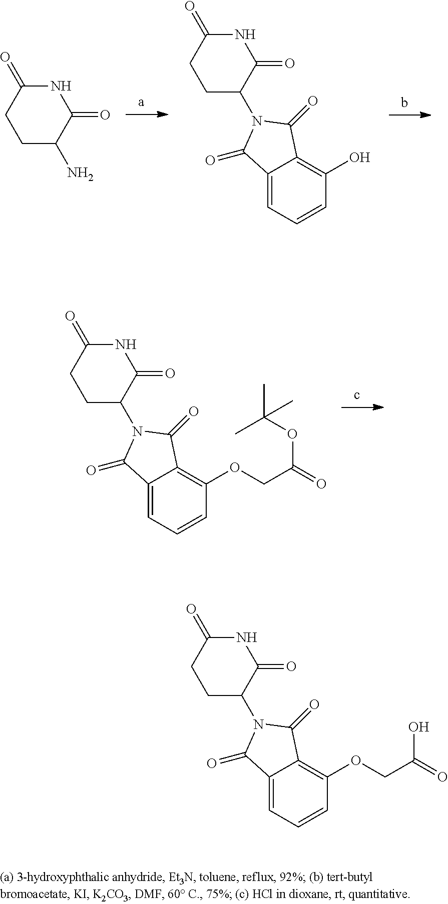 [SMILES: NC1CCC(=O)NC1=O ; O=C1CCC(N2C(=O)c3cccc(O)c3C2=O)C(=O)N1 ; CC(C)(C)OC(=O)COc1cccc2c1C(=O)N(C1CCC(=O)NC1=O)C2=O ; O=C(O)COc1cccc2c1C(=O)N(C1CCC(=O)NC1=O)C2=O]

### 2-(2,6-Dioxopiperidin-3-yl)-4-hydroxyisoindoline-1,3-dione

To a mixture of 3-hydroxyphthalic anhydride (420 mg, 2.5 mmol) and 3-aminopiperidine-2,6-dione hydrochloride (420 mg, 2.5 mmol) was added dry toluene (20 mL) and triethylamine (0.4 mL, 2.8 mmol). The resulting solution was refluxed for 18 hours. The reaction mixture was concentrated and purified by silica gel column chromatography (0-10% MeOH in CHCl₃) to obtain the title compound as a yellowish solid (630 mg, 92%). ¹H NMR (400 MHz, DMSO-d₆) δ 11.19 (s, 1H), 11.10 (s, 1H), 7.66 (dd, J=7.2, 8.4 Hz, 1H), 7.33 (d, J=6.8 Hz, 1H), 7.26 (d, J=8.4 Hz, 1H), 5.08 (dd, J=5.2, 12.8 Hz, 1H), 2.95-2.84 (m, 1H), 2.64-2.52 (m, 2H), 2.07-1.98 (m, 1H).

### tert-Butyl 2-(2-(2,6-dioxopiperidin-3-yl)-1,3-dioxoisoindolin-4-yloxy)acetate

To a mixture of 2-(2,6-dioxopiperidin-3-yl)-4-hydroxyisoindoline-1,3-dione (170 mg, 0.62 mmol), tert-butyl bromoacetate (150 mg, 0.77 mmol) and K₂CO₃ (110 mg, 0.79 mmol) was added dry DMF (2 mL) and KI (10 mg, 0.06 mmol). The mixture stirred at 60° C. for 18 hours. The reaction mixture was concentrated, diluted with EtOAc (50 mL), washed with water (10 mL) and brine (10 mL). The organic phase was dried over Na₂SO₄, concentrated, and purified by silica gel column chromatography (0-3% MeOH in CHCl₃) to obtain the title compound as a white solid (180 mg, 75%). ¹H NMR (400 MHz, DMSO-d₆) δ 11.12 (s, 1H), 7.83-7.77 (m, 1H), 7.49 (d, J=6.8 Hz, 1H), 7.39 (d, J=8.4 Hz, 1H), 5.14-5.08 (dd, J=5.2, 12.8 Hz, 1H), 4.97 (s, 2H), 2.96-2.84 (m, 1H), 2.64-2.52 (m, 2H), 2.08-1.99 (m, 1H), 1.43 (s, 9H).

### 2-(2-(2,6-Dioxopiperidin-3-yl)-1,3-dioxoisoindolin-4-yloxy)acetic acid

tert-Butyl 2-(2-(2,6-dioxopiperidin-3-yl)-1,3-dioxoisoindolin-4-yloxy)acetate (220 mg, 0.57 mmol) was dissolved in 4 M HCl in dioxane (3 mL, 12 mmol) and stirred at rt for 3 hours. The reaction mixture was concentrated to dryness and co-evaporated with methanol (2×5 mL) to obtain the title compound as a white solid (190 mg, quantitative). ¹H NMR (500 MHz, DMSO-d₆) δ 13.24 (bs, 1H), 11.10 (s, 1H), 7.79 (dd, J=7.0, 9.0 Hz, 1H), 7.48 (d, J=7.0 Hz, 1H), 7.40 (d, J=9.0 Hz, 1H), 5.11 (dd, J=5.5, 12.0 Hz, 1H), 4.99 (s, 2H), 2.94-2.84 (m, 1H), 2.64-2.52 (m, 2H), 2.07-2.01 (m, 1H).

### Example 3: Boc-Protection and Mesylation

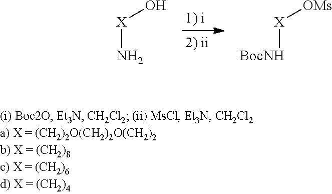

### 2-(2-(2-tert-Butoxycarbonylaminoethoxy)ethoxy)ethyl methanesulfonate

To a cooled solution of 6-amino hexanol (300 mg, 2.0 mmol) in CH₂Cl₂ (2.5 mL) was added Boc-anhydride (450 mg, 2.0 mmol) in CH₂Cl₂ (2.5 mL) and stirred for 18 h. The reaction mixture was concentrated and purified by silica gel column chromatography (20-100% EtOAc in hexanes) to obtain 2-(2-(2-tert-butoxy-carbonylaminoethoxy)ethoxy)ethanol as yellow oil (430 mg, 86%).

To a cooled solution of 2-(2-(2-tert-butoxycarbonylaminoethoxy)ethoxy)ethanol (300 mg, 1.2 mmol) in CH₂Cl₂ (5 mL) was added triethylamine (0.4 mL, 2.8 mmol), methanesulfonylchloride (0.15 mL, 1.9 mmol) and a catalytic amount of 4-dimethylaminopyridine (10 mg). The reaction mixture stirred for 2 hours and then was diluted with CHCl₃ (50 mL), washed with saturated NaHCO₃ solution (10 mL) and saturated brine (10 mL) solution. The organic phase was concentrated and purified by silica gel column chromatography (0-80% EtOAc in hexanes) to obtain the title compound as a yellow oil (400 mg, quantitative). ¹H NMR (400 MHz, CDCl₃) δ 4.94 (bs, 1H), 4.42-4.37 (m, 2H), 3.79-3.75 (m, 2H), 3.69-3.65 (m, 2H), 3.64-3.59 (m, 2H), 3.53 (t, J=5.2 Hz, 2H), 3.35-3.28 (m, 2H), 3.07 (s, 3H), 1.44 (s, 9H).

### 8-(tert-Butoxycarbonylamino)octyl methanesulfonate

To a cooled solution of 8-amino octanol (210 mg, 1.5 mmol) in CH₂Cl₂ (5 mL) was added Boc-anhydride (360 mg, 1.65 mmol) in CH₂Cl₂ (2.5 mL) and stirred for 3 hours. The reaction mixture was concentrated and purified by silica gel column chromatography (0-60% EtOAc in hexanes) to obtain 8-(tert-butoxycarbonyl-amino)octan-1-ol as a white solid (265 mg, 72%).

To a cooled solution of 8-(tert-butoxycarbonylamino)octan-1-ol (250 mg, 1.02 mmol) in CH₂Cl₂ (2.5 mL) was added triethylamine (0.4 mL, 2.8 mmol), methanesulfonylchloride (175 mg, 1.53 mmol) and a catalytic amount of 4-dimethylaminopyridine (20 mg). The reaction mixture stirred for 2 hours and then was diluted with CHCl₃ (50 mL), washed with saturated NaHCO₃ solution (10 mL) and saturated brine (10 mL) solution. The organic phase was concentrated and purified silica gel column chromatography (0-50% EtOAc in hexanes) to obtain the title compound as a clear oil (280 mg, 86%). ¹H NMR (500 MHz, CDCl₃) δ 4.50 (bs, 1H), 4.22 (t, J=6.5 Hz, 2H), 3.14-3.07 (m, 2H), 3.00 (s, 3H), 1.78-1.71 (m, 2H), 1.51-1.36 (m, 13H), 1.36-1.28 (m, 6H).

### 6-(tert-Butoxycarbonylamino)hexyl methanesulfonate

To a cooled solution of 6-amino hexanol (350 mg, 3.0 mmol) in CH₂Cl₂ (2.5 mL) was added Boc-anhydride (650 mg, 3.0 mmol) in CH₂Cl₂ (2.5 mL) and stirred for 18 hours. The reaction mixture was concentrated and purified by silica gel column chromatography (0-80% EtOAc in hexanes) to obtain 6-(tert-butoxycarbonyl-amino)hexan-1-ol as a white solid (640 mg, 100%).

To a cooled solution of 6-(tert-butoxycarbonylamino)hexan-1-ol (420 mg, 2.0 mmol) in CH₂Cl₂ (5 mL) was added triethylamine (0.8 mL, 5.6 mmol), methanesulfonylchloride (0.25 mL, 3.2 mmol) and a catalytic amount of 4-dimethylaminopyridine (20 mg). The reaction mixture stirred for 2 hours and then was diluted with CHCl₃ (50 mL), washed with saturated NaHCO₃ solution (10 mL) and saturated brine (10 mL) solution. The organic phase was concentrated and purified silica gel column chromatography (0-50% EtOAc in hexanes) to obtain the title compound as a white solid (490 mg, 88%). ¹H NMR (400 MHz, CDCl₃) δ 4.52 (bs, 1H), 4.22 (t, J=6.4 Hz, 2H), 3.16-3.08 (m, 2H), 3.00 (s, 3H), 1.80-1.71 (m, 2H), 1.54-1.46 (m, 2H), 1.44 (s, 9H), 1.43-1.31 (m, 4H).

### Example 4: Synthesis of 2-(2-(2,6-dioxopiperidin-3-yl)-1,3-dioxoisoindolin-4-yloxy)-N-(6-(2-((1E,3E)-4-(6-(methylamino)pyridin-3-yl)buta-1,3-dienyl)benzo[d]thiazol-6-yloxy)hexyl)acetamide (2)

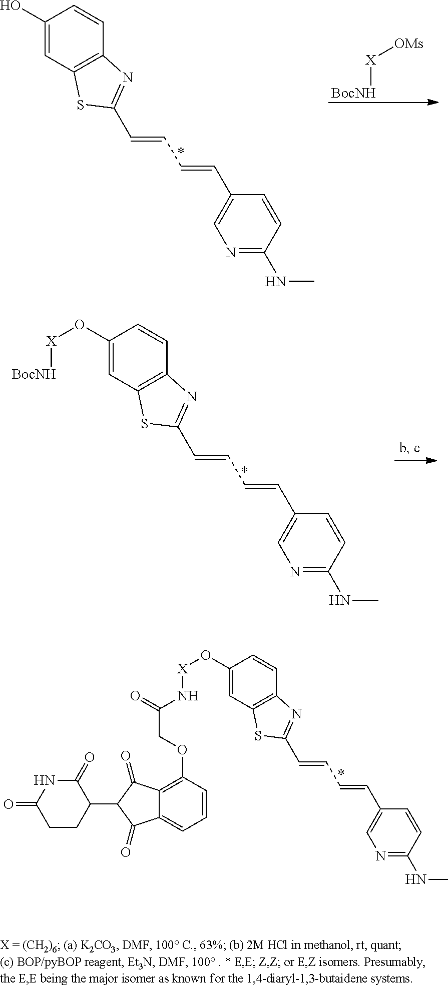

### tert-Butyl 6-(2-((1E,3E)-4-(6-(methylamino)pyridin-3-yl)buta-1,3-dienyl)benzo[d]thiazol-6-yloxy)hexyl-carbamate

To a solution of 2-((1E,3E)-4-(6-(methylamino)pyridin-3-yl)buta-1,3-dienyl)benzo[d]thiazol-6-ol (110 mg, 0.35 mmol) in DMF (2.5 mL) was added K₂CO₃ (100 mg, 0.71 mmol) and 6-(tert-butoxycarbonylamino)hexyl methanesulfonate (133 mg, 0.45 mmol) and the reaction mixture stirred at 70° C. for 6 hours. The reaction mixture was concentrated and then diluted with CHCl₃ (20 mL) and water (2 mL). The organic layer was washed with saturated brine solution (10 mL), dried over Na₂SO₄, concentrated and purified by silica gel column chromatography (0-40% acetone in hexanes) to obtain the title compound as a yellow solid (115 mg, 63%). MS (ESI⁺) m/z 509.3 (M+H)⁺.

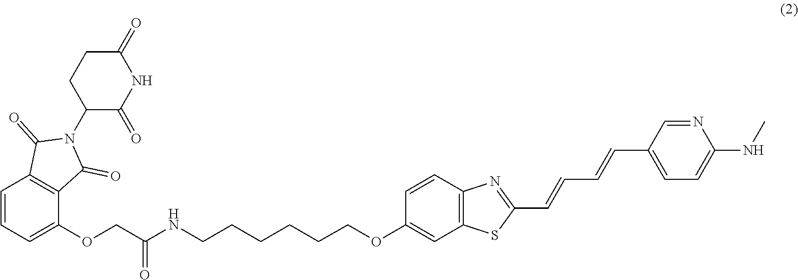 [SMILES: CNc1ccc(/C=C/C=C/c2nc3ccc(OCCCCCCNC(=O)COc4cccc5c4C(=O)N(C4CCC(=O)NC4=O)C5=O)cc3s2)cn1]

### 2-(2-(2,6-Dioxopiperidin-3-yl)-1,3-dioxoisoindolin-4-yloxy)-N-(6-(2-((1E,3E)-4-(6-(methylamino)pyridin-3-yl)buta-1,3-dienyl)benzo[d]thiazol-6-yloxy)hexyl)acetamide

To a cooled solution of tert-butyl 6-(2-((1E,3E)-4-(6-(methylamino)pyridin-3-yl)buta-1,3-dienyl)benzo[d]thiazol-6-yloxy)hexyl-carbamate (100 mg, 200 umol) in MeOH (0.5 mL) was added 2 M HCl in methanol (2.5 mL, 5.0 mmol) and stirred at rt for 3 hours. The reaction mixture was concentrated to dryness and co-evaporated with methanol (2×5 mL) to obtain yellow solid residue. To the residue was added 2-(2-(2,6-dioxopiperidin-3-yl)-1,3-dioxoisoindolin-4-yloxy)acetic acid (60 mg, 180 μmol), DMF (2.0 mL), triethylamine (100 mg, 1.0 mmol), and pyBOP reagent (100 mg, 192 μmol). The solution stirred at rt for 6 hours and then concentrated to dryness, and triturated with water and then hexanes. The resulting residue was diluted with CHCl₃ (50 mL) and washed with water (2×10 mL). The organic layer was dried over Na₂SO₄, concentrated and purified by silica gel column chromatography (0-10% MeOH in CHCl₃) to obtain the title compound as a yellow solid (130 mg, 100%).

### Example 5: Synthesis of 2-(2-(2,6-dioxopiperidin-3-yl)-1,3-dioxoisoindolin-4-yloxy) -N-(6-(2-((1E,3E)-4-(6-(methylamino)pyridin-3-yl)buta-1,3-dienyl)benzo[d]thiazol-6-yloxy) octyl)acetamide (3)

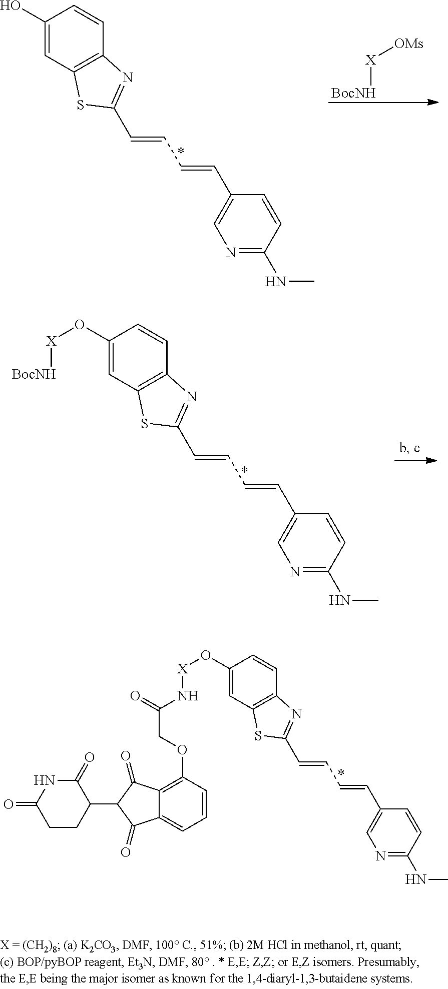

### tert-Butyl 6-(2-((1E,3E)-4-(6-(methylamino)pyridin-3-yl)buta-1,3-dienyl)benzo[d]thiazol-6-yloxy)octyl-carbamate

To a solution of 2-((1E,3E)-4-(6-(Methylamino)pyridin-3-yl)buta-1,3-dienyl)benzo[d]thiazol-6-ol (95 mg, 0.30 mmol) in DMF (2.5 mL) was added Cs₂CO₃ (180 mg, 0.55 mmol), and 8-(tert-butoxycarbonylamino)octyl methanesulfonate (130 mg, 0.40 mmol) and the reaction mixture stirred at 70° C. for 6 hours. The reaction mixture was concentrated and then diluted with CHCl₃ (20 mL) and water (2 mL). The organic layer was washed with saturated brine solution (10 mL), dried over Na₂SO₄, concentrated and purified by silica gel column chromatography (0-40% acetone in hexanes) to obtain the title compound as a yellow solid (86 mg, 51%). (400 MHz, DMSO-d₆) δ 8.09 (d, J=2.0 Hz, 1H), 7.78 (d, J=9.2 Hz, 1H), 7.70 (dd, J=2.0, 8.98 Hz, 1H), 7.60 (d, J=2.4 Hz, 1H), 7.27 (dd, J=9.6, 15.2 Hz, 1H), 7.05 (dd, J=2.4, 8.8 Hz, 1H), 6.98-6.82 (m, 4H), 6.76 (t, J=5.2 Hz, 1H), 6.49 (d, J=8.8 Hz, 1H), 4.02 (t, J=6.4 Hz, 2H), 2.92-2.86 (m, 2H), 2.80 (d, J=4.8 Hz, 3H), 1.77-1.69 (m, 2H), 1.48-1.19 (m, 19H).

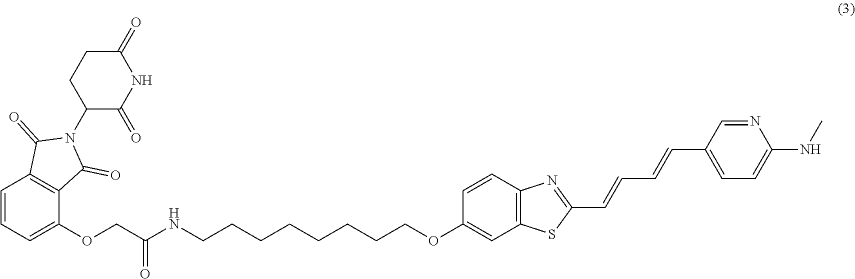 [SMILES: CNc1ccc(/C=C/C=C/c2nc3ccc(OCCCCCCCCNC(=O)COc4cccc5c4C(=O)N(C4CCC(=O)NC4=O)C5=O)cc3s2)cn1]

### 2-(2-(2,6-Dioxopiperidin-3-yl)-1,3-dioxoisoindolin-4-yloxy)-N-(6-(2-((1E,3E)-4-(6-(methylamino)pyridin-3-yl)buta-1,3-dienyl)benzo[d]thiazol-6-yloxy)octyl)acetamide

To a cooled solution of tert-butyl 6-(2-((1E,3E)-4-(6-(methylamino)pyridin-3-yl)buta-1,3-dienyl)benzo[d]thiazol-6-yloxy)octyl-carbamate (80 mg, 150 umol) in MeOH (1.0 mL) was added 2 M HCl in methanol (2.0 mL, 4.0 mmol) and stirred at rt for 3 hours. The reaction mixture was concentrated to dryness and co-evaporated with methanol (2×5 mL) to obtain yellow solid residue. To the residue was added 2-(2-(2,6-dioxopiperidin-3-yl)-1,3-dioxoisoindolin-4-yloxy)acetic acid (50 mg, 155 μmol), DMF (2.0 mL), triethylamine (140 mg, 1.4 mmol), and pyBOP reagent (88 mg, 170 μmop. The solution stirred at rt for 6 hours and then concentrated to dryness, triturated with water and hexanes successively. The resulting residue was diluted with CHCl₃ (50 mL) and washed with water (2×10 mL). The organic layer was dried over Na₂SO₄, concentrated and purified by silica gel column chromatography (0-10% MeOH in CHCl₃) to obtain the title compound as a yellow solid (90 mg, 80%) as a yellow solid.

### Example 6: Synthesis of 2-(2-(2,6-dioxopiperidin-3-yl)-1,3-dioxoisoindolin-4-yloxy)-N-(2-(2-(2-(2-((1E,3E)-4-(6-(methylamino)pyridin-3-yl)buta-1,3-dienyl)benzo[d]thiazol-6-yloxy)ethoxy)ethoxy)ethyl)acetamide (4)

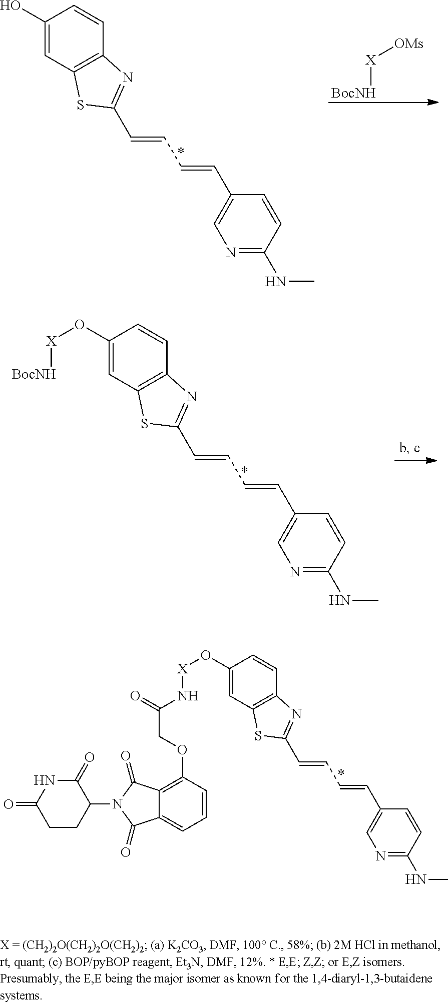

### tert-Butyl-2-(2-(2-(2-((1E,3E)-4-(6-(methylamino)pyridin-3-yl)buta-1,3-dienyl)benzo[d]thiazol-6-yloxy)ethoxy)ethoxy)ethylcarbamate

To a solution of 2-((1E,3E)-4-(6-(Methylamino)pyridin-3-yl)buta-1,3-dienyl)benzo[d]thiazol-6-ol (25 mg, 0.08 mmol) in DMF (2.0 mL) was added K₂CO₃ (28 mg, 0.20 mmol), and 2-(2-(2-tert-butoxycarbonylaminoethoxy)ethoxy)ethyl methanesulfonate (31 mg, 0.09 mmol) and the reaction mixture stirred at 100° C. for 3 hours and then allowed to cool to rt over 18 hours. The reaction mixture was concentrated and then diluted with CHCl₃ (20 mL) and water (2 mL). The organic layer was washed with saturated brine solution (10 mL), dried over Na₂SO₄, concentrated and purified by preparative TLC (8% MeOH in CHCl₃) to obtain the title compound as a yellow solid (25 mg, 58%). MS (ESI⁺) m/z 541.5 (M+H)⁺.

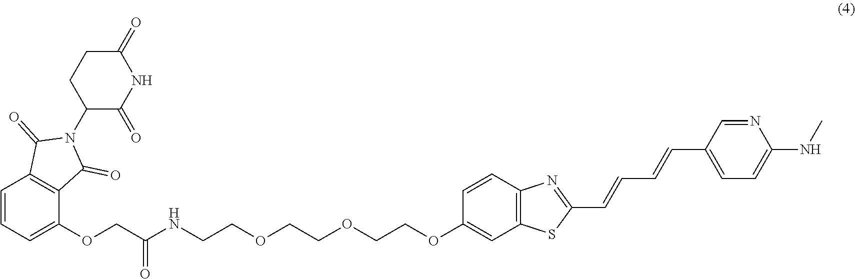 [SMILES: CNc1ccc(/C=C/C=C/c2nc3ccc(OCCOCCOCCNC(=O)COc4cccc5c4C(=O)N(C4CCC(=O)NC4=O)C5=O)cc3s2)cn1]

### 2-(2-(2,6-Dioxopiperidin-3-yl)-1,3-dioxoisoindolin-4-yloxy)-N-(2-(2-(2-(2-((1E,3E)-4-(6-(methylamino)pyridin-3-yl)buta-1,3-dienyl)benzo thiazol-6-yloxy)ethoxy)ethoxy)ethyl)acetamide

To a cooled solution of tert-butyl-2-(2-(2-(2-((1E,3E)-4-(6-(methylamino)pyridin-3-yl)buta-1,3-dienyl)benzo[d]thiazol-6-yloxy)ethoxy)ethoxy)ethylcarbamate (25 mg, 46 μmol) in MeOH (1.0 mL) was added 2 M HCl in methanol (2.0 mL, 8.0 mmol) and stirred at rt for 3 hours. The reaction mixture was concentrated to dryness and co-evaporated with methanol (2×5 mL) to obtain yellow solid residue. To the residue were added pyBOP reagent (27 mg, 61 μmol), a solution of 2-(2-(2,6-dioxopiperidin-3-yl)-1,3-dioxoisoindolin-4-yloxy)acetic acid (16 mg, 50 μmol) in DMF (1.0 mL) and triethylamine (20 mg, 200 μmol). The solution was stirred at rt for 18 hours and then concentrated to dryness. The crude product was purified by preparative TLC (10% MeOH in CHCl₃) to obtain the title compound as a yellow solid (35 mg), which was further purified by preparative HPLC (74:18:8% Heptane:IPA:MeCN, Viridis® 2-EthylPyridine column) to the obtain the title compound as a yellow solid (4.2 mg, 12%). MS (ESI⁺) m/z 755.1 (M+H)⁺.

### Example 7: Dose-Effect on Tau of A152T and P301L Neurons

Assays were performed according to protocols described in Silva et al., eLife, 8:e45457 (2019). Each of the bispecific compounds, along with the control parental scaffold, was evaluated for the ability to degrade tau protein in cultured human differentiated frontotemporal lobar dementia (FTD) neurons.

For each condition, 1 well of differentiated neuronal cells (6 well-plate) was washed and collected in PBS, pelleted and lysed in radioimmunoprecipitation assay (RIPA) buffer (Boston BioProducts) with 2% SDS (Sigma), protease inhibitors (Roche cOmplete™ Mini tablets), and phosphatase inhibitors (Sigma), followed by sonication in a water sonicator (Bransonic® Ultrasonic Baths, Thomas Scientific) for 5 minutes, and centrifugation at 20,000 g for 15 minutes. Supernatants were transferred to new tubes, total protein concentration was quantified with the Pierce™ BCA Protein Assay Kit (Thermo Scientific™), and SDS-PAGE gels were loaded with 10 μg total protein per well (pre-boiled samples). Western blots were performed with the Novex NuPAGE™ SDS-PAGE Gel System (Invitrogen™). All samples were resolved in 7% Tris-Acetate gels with Tris-Acetate running buffer (Invitrogen™). Blots were probed with antibodies against total tau (TAU5) and phosphorylated tau (Ser396), along with (3-actin as a loading control; followed by corresponding HRP-linked secondary antibodies (Cell Signaling Technology®), and SuperSignal™ West Pico Chemiluminescent Substrate (Thermo Scientific™) detection. Membranes were exposed to autoradiographic film (LabScientific) and films were scanned using an Epson® Perfection V800 Photo Scanner. Protein band intensities (pixel mean intensity) were quantified using Adobe Photoshop® CS5 Histogram function.

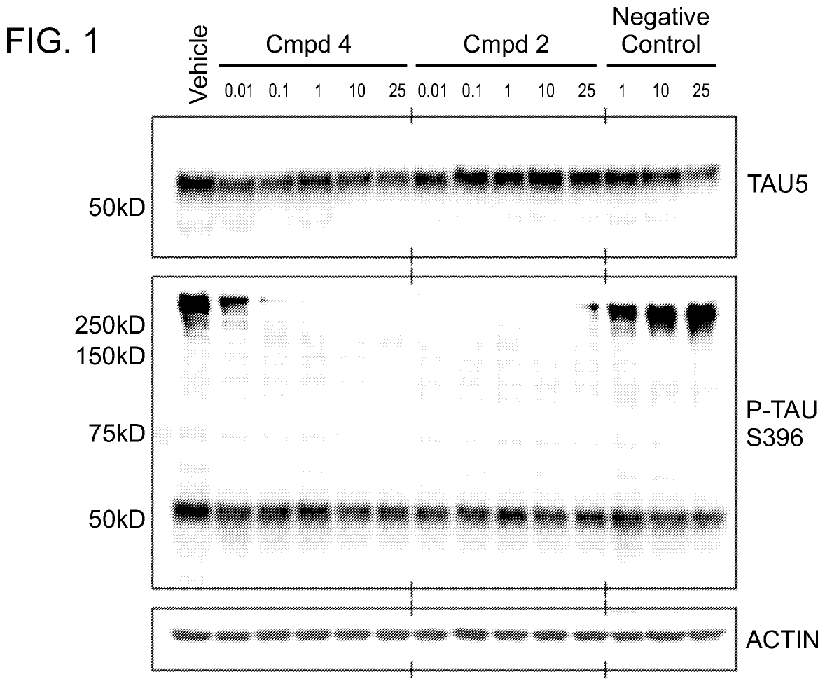

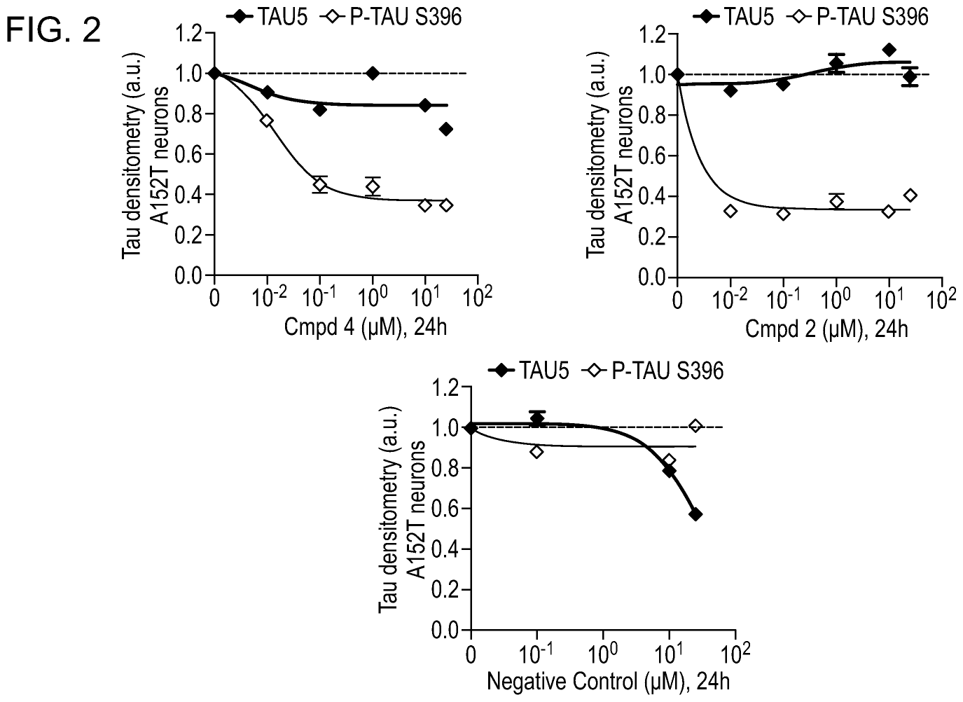

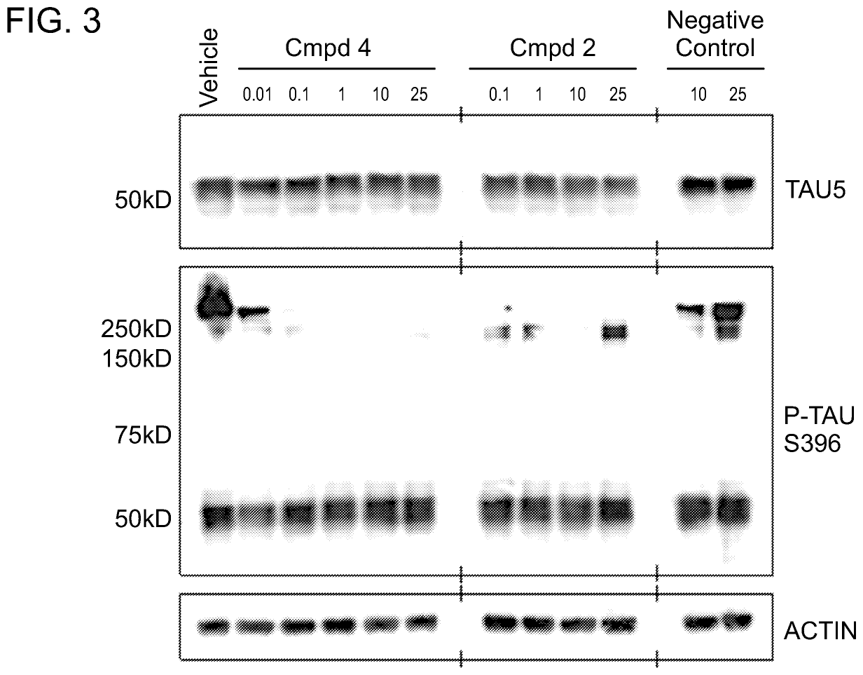

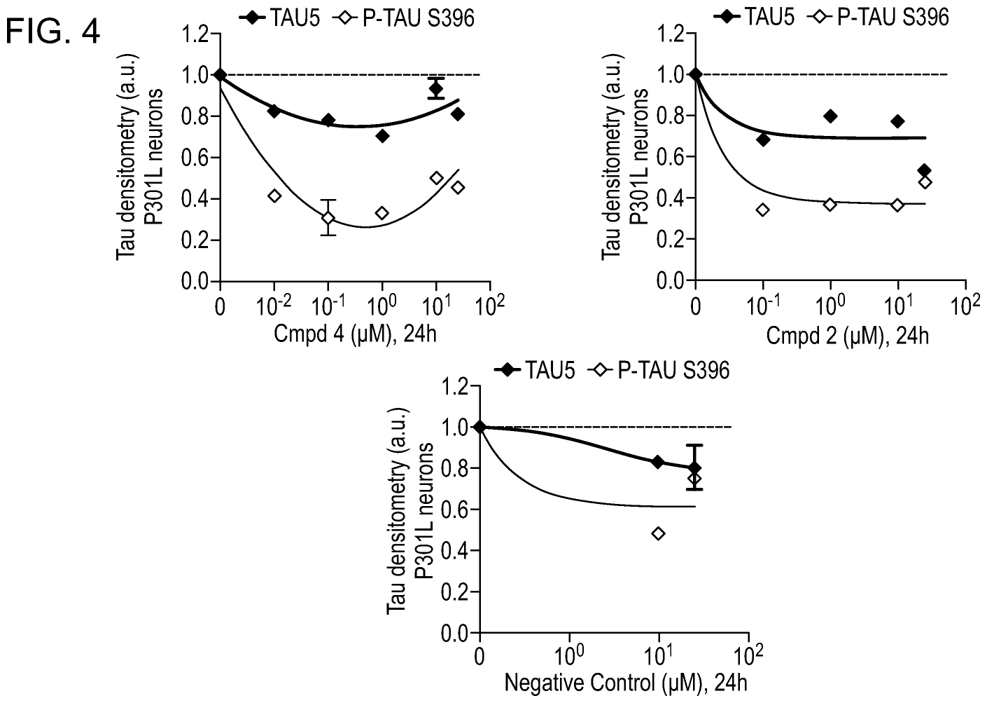

The vehicle alone (DMSO) had no effect on tau. However, varying degrading activity was observed among the specific compounds with overall similar relative levels of degrading activity between tau-A152T neurons and tau-P301L neurons. Bispecific compounds were added at the doses indicated (either 1 μM or 10 μM). Lenalidomide (1 μM or 10 μM) was also evaluated as a control since it binds CRBN but did not engage tau, as confirmed by having no effect on tau levels. Bispecific compounds 2 and 4 significantly degraded hyperphosphorylated tau and/or total tau in human tau-A152T neurons (6-week differentiated) and tau-P301L neurons (6-week differentiated) after treatment of the neurons with the bispecific compounds for 24 hours (FIG. 1-FIG. 4).

All patent publications and non-patent publications are indicative of the level of skill of those skilled in the art to which this invention pertains. All these publications are herein incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

What is claimed is:

1. A bispecific compound having a structure represented by formula (I): 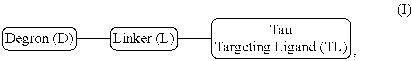
wherein the targeting ligand represents a moiety that binds tau, the degron represents a moiety that binds an E3 ubiquitin ligase or the degron is an autophagy-recruiting tag, and the linker represents a moiety that covalently connects the degron and the targeting ligand, or a pharmaceutically acceptable salt or stereoisomer thereof,
wherein 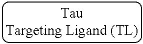
is represented by the formula TL-1 or TL-2: 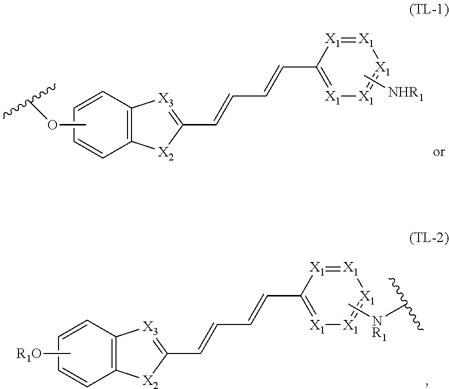
wherein
each X₁ is independently C, CH, or N;
X₂ is NH, S, or O;
X₃ is CH or N; and
each R₁ is independently hydrogen or C₁-C₃ alkyl,
provided that when X₂ is NH, X₃ is CH; and
further provided that when X₃ is N, X₂ is S or O.

2. The bispecific compound of claim 1, wherein TL-1 is represented by any one of structures TL-1a to TL-1o:

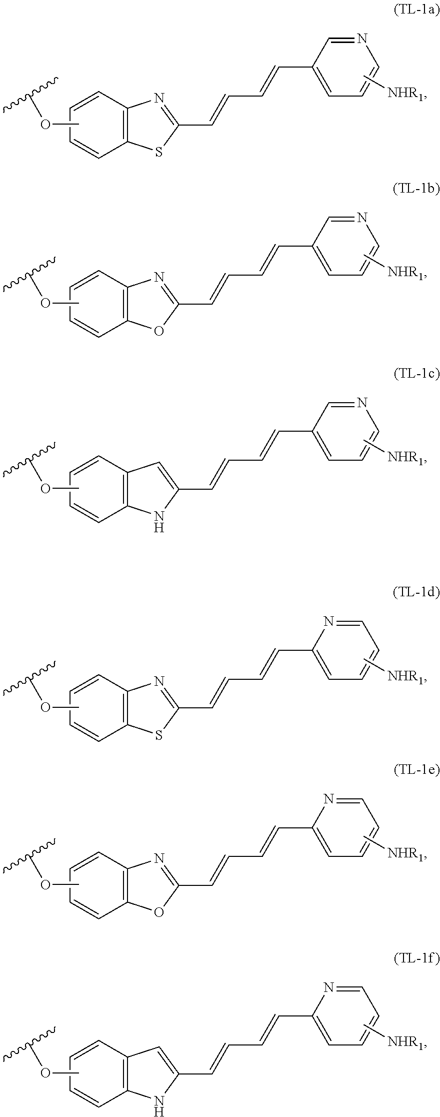

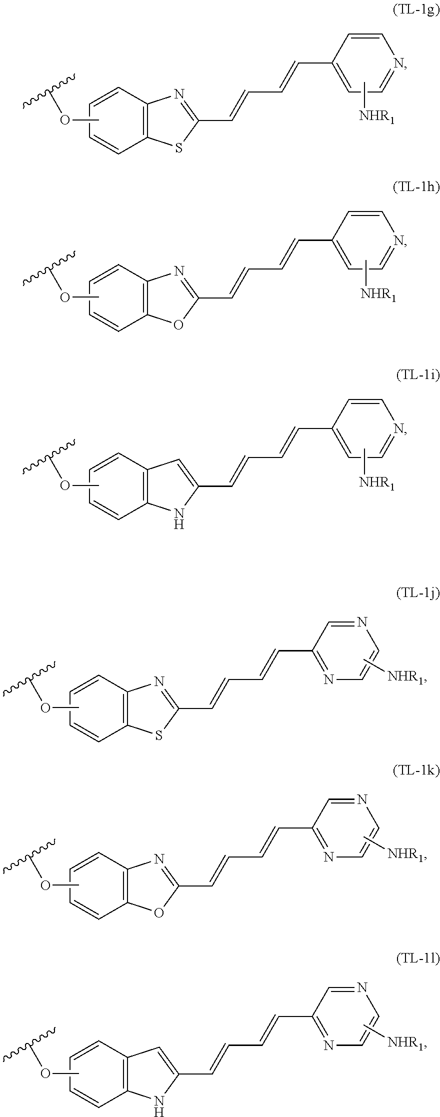

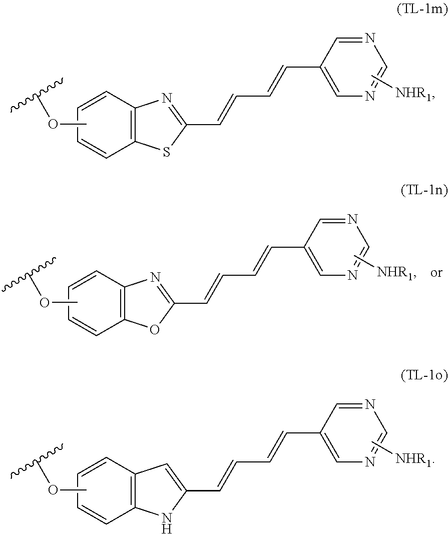

3. The bispecific compound of claim 2, wherein TL-1

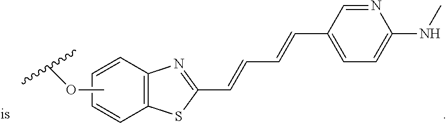

4. The bispecific compound of claim 1, wherein TL-2 is represented by any one of structures TL-2a to TL-2o:

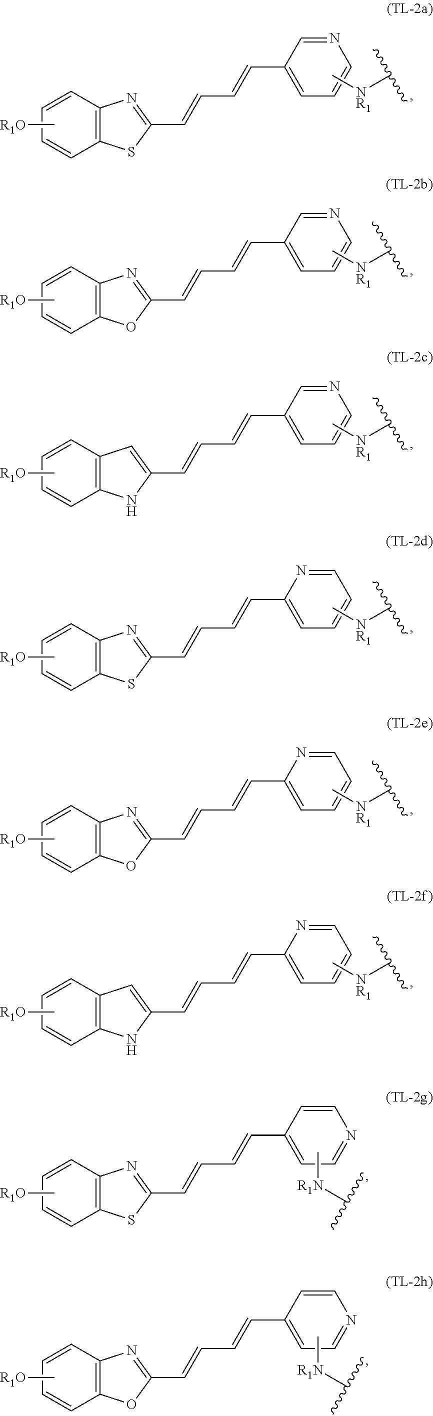

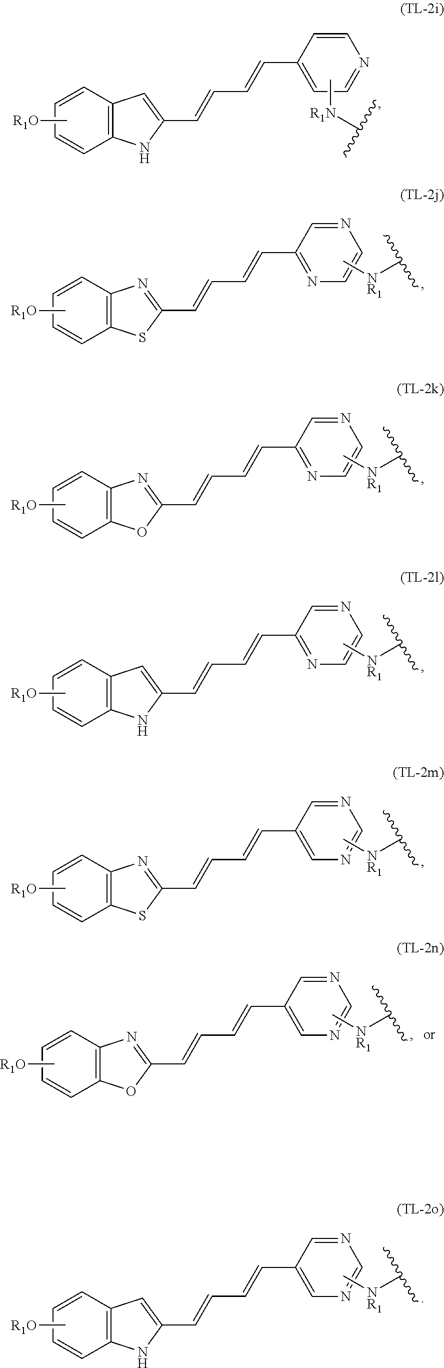

5. The bispecific compound of claim 1, wherein R₁ is hydrogen.

6. The bispecific compound of claim 1, wherein R₁ is methyl.

7. The bispecific compound of claim 1, wherein the linker comprises an alkylene chain or a bivalent alkylene chain, either of which is optionally interrupted by, and/or terminate at either or both termini in at least one of —O—, —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, C₃-C₁₂ carbocyclene, 3- to 12-membered heterocyclene, 5- to 12-membered heteroarylene or any combination thereof, wherein R′ is H or C₁-C₆ alkyl, wherein the interrupting and the one or both terminating groups may be the same or different.

8. The bispecific compound of claim 7, wherein the linker comprises an alkylene chain having 1-10 alkylene units and is interrupted by and/or terminates in

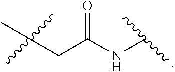 [SMILES: *CC(=O)N*]

9. The bispecific compound of claim 1, wherein the linker comprises a polyethylene glycol chain which optionally may terminate at either or both termini in at least one of —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, C₃-12 carbocyclene, 3- to 12-membered heterocyclene, 5- to 12-membered heteroarylene or any combination thereof, wherein R′ is H or C₁-C₆ alkyl, wherein the one or both terminating groups may be the same or different.

10. The bispecific compound of claim 9, wherein the linker comprises a polyethylene glycol chain having 2-8 PEG units and terminating in

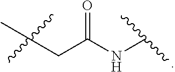 [SMILES: *CC(=O)N*]

11. The bispecific compound of claim 1, which is represented by any one of the following structures: 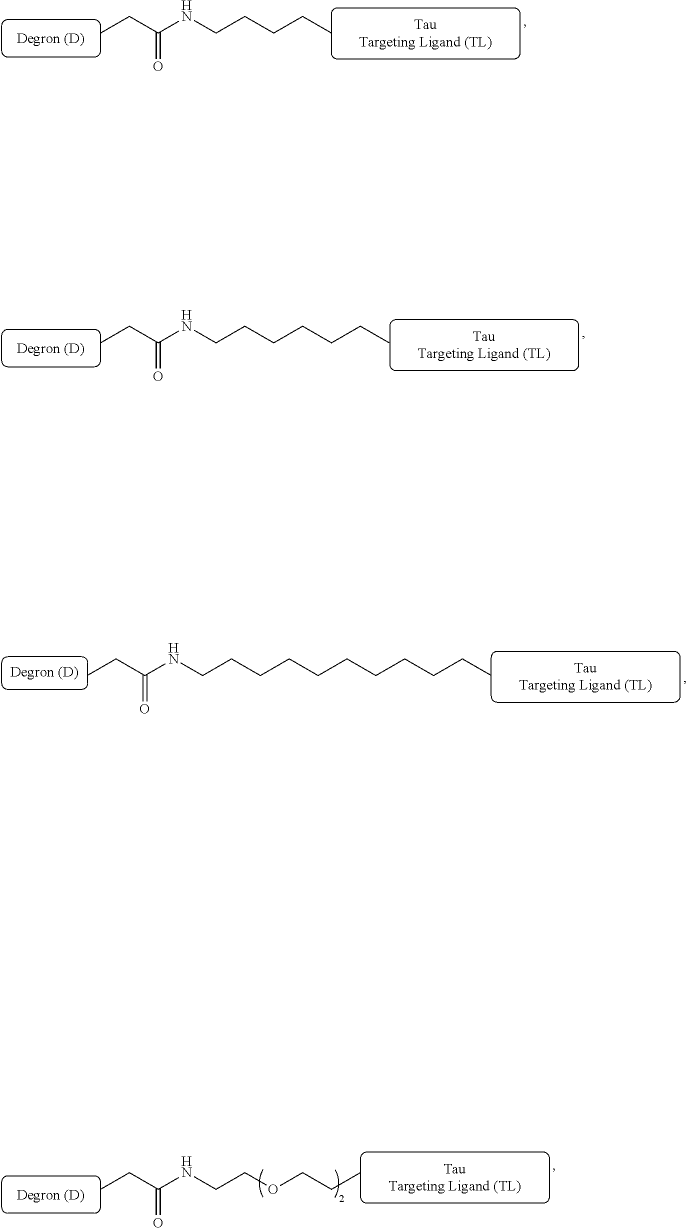
or a pharmaceutically acceptable salt or stereoisomer thereof.

12. The bispecific compound of claim 1, wherein the degron binds cereblon and the degron is represented by any one of structures (D1-a-D1-i): 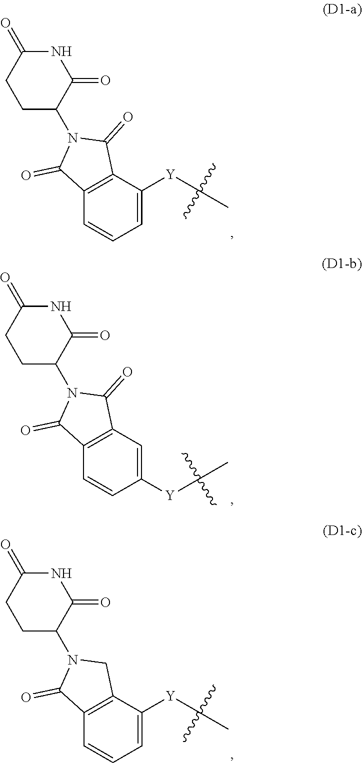 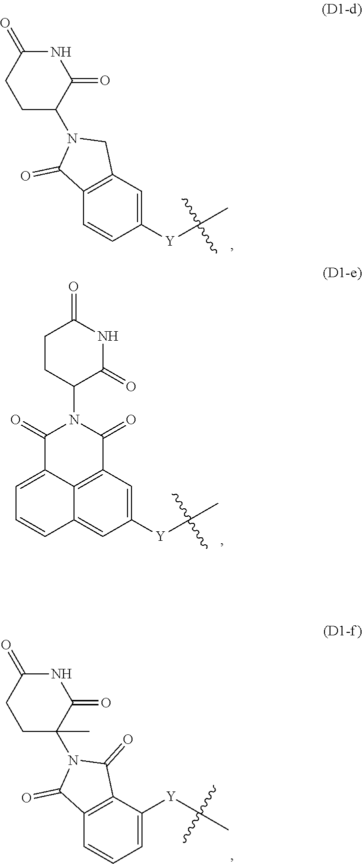 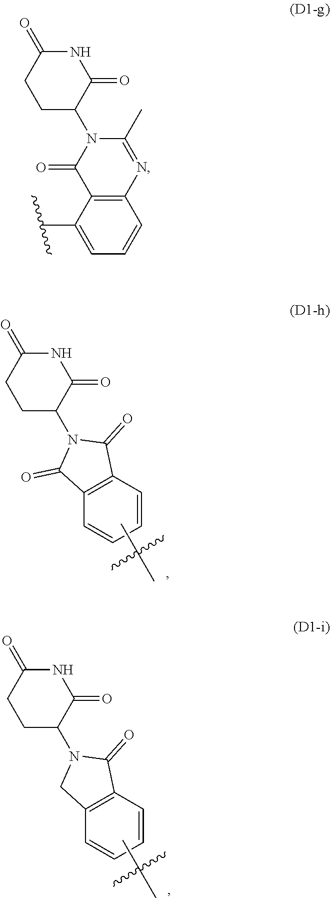
wherein
Y is NH or O.

13. (canceled)

14. The bispecific compound of claim 1, wherein the degron binds von Hippel-Landau (VHL) and the degron is represented by any one of structures D2-a-D2-d: 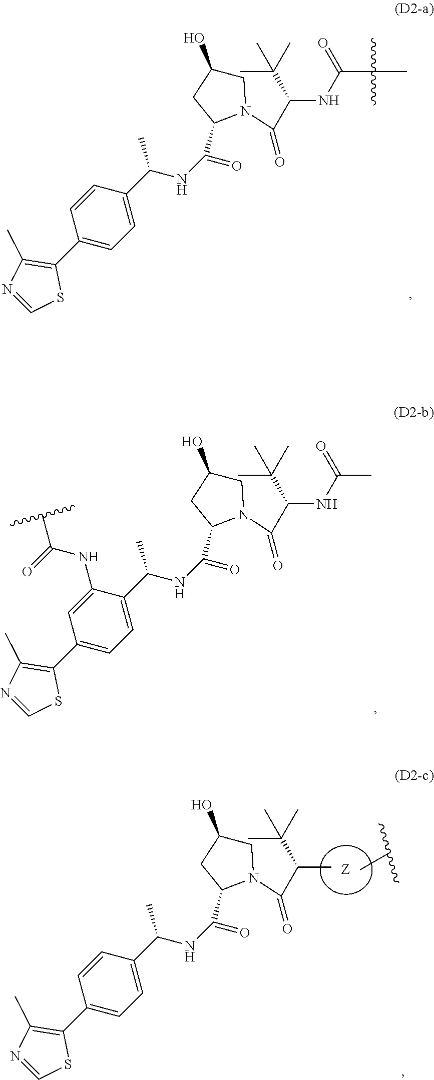
wherein Z is a cyclic group, or 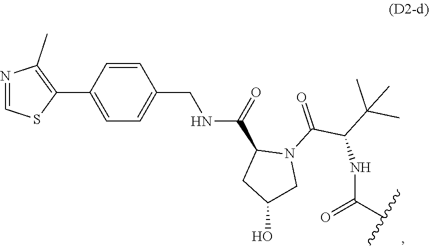 [SMILES: *C(=O)N[C@H](C(=O)N1C[C@H](O)C[C@H]1C(=O)NCc1ccc(-c2scnc2C)cc1)C(C)(C)C]
or a stereoisomer thereof.

15. (canceled)

16. The bispecific compound of claim 1, wherein the degron binds an inhibitor of apoptosis protein (TAP) and the degron is represented by any one of structures D3-a-D3-d: 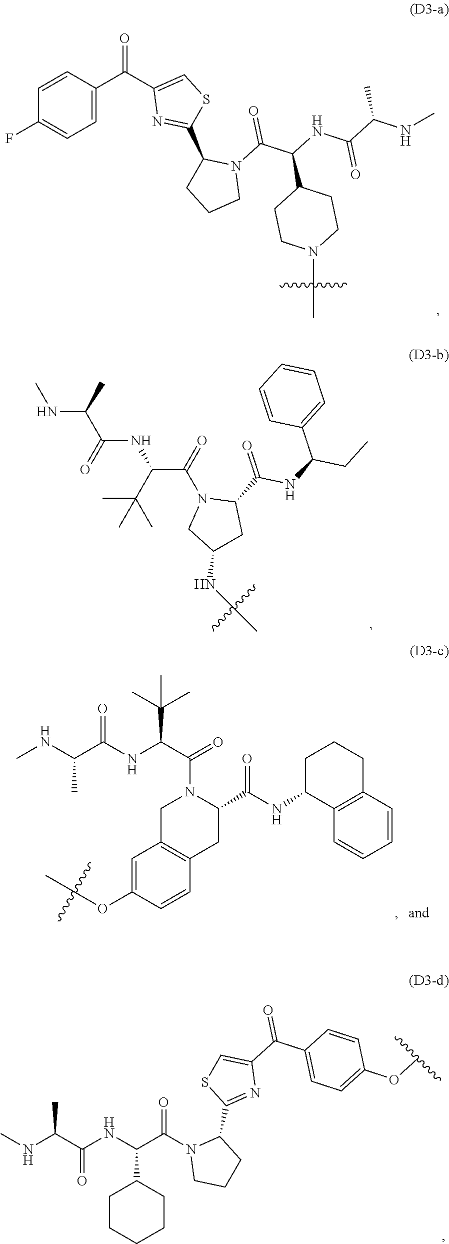
or a stereoisomer thereof.

17. (canceled)

18. The bispecific compound of claim 1, wherein the degron binds murine double minute 2 (MDM2) and the degron is represented by any one of structures D4-a-D4-b: 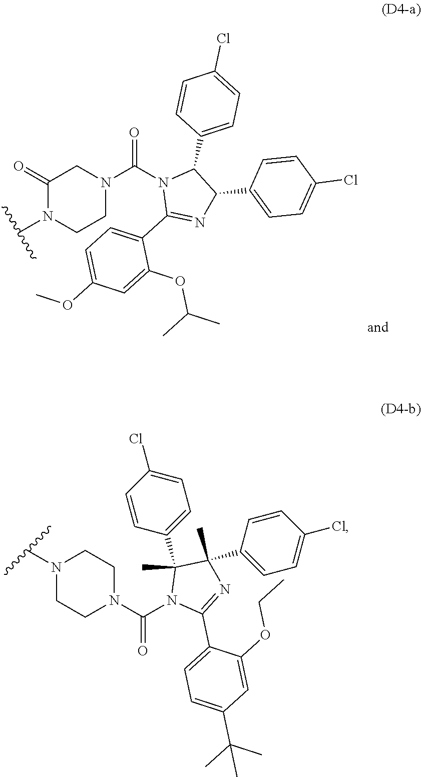 [SMILES: *N1CCN(C(=O)N2C(c3ccc(OC)cc3OC(C)C)=N[C@@H](c3ccc(Cl)cc3)[C@H]2c2ccc(Cl)cc2)CC1=O ; *N1CCN(C(=O)N2C(c3ccc(C(C)(C)C)cc3OCC)=N[C@@](C)(c3ccc(Cl)cc3)[C@@]2(C)c2ccc(Cl)cc2)CC1]
or a stereoisomer thereof.

19. (canceled)

20. The bispecific compound of claim 1, wherein the degron is an autophagy-recruiting tag and the degron is represented by either of structures D5-a-D5-b: 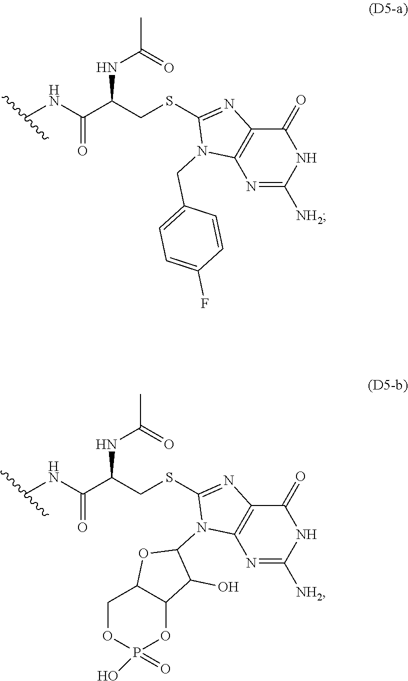 [SMILES: *NC(=O)[C@H](CSc1nc2c(=O)[nH]c(N)nc2n1Cc1ccc(F)cc1)NC(C)=O ; *NC(=O)[C@H](CSc1nc2c(=O)[nH]c(N)nc2n1C1OC2COP(=O)(O)OC2C1O)NC(C)=O]
or a stereoisomer thereof.

21. (canceled)

22. The bispecific compound of claim 1, which is: 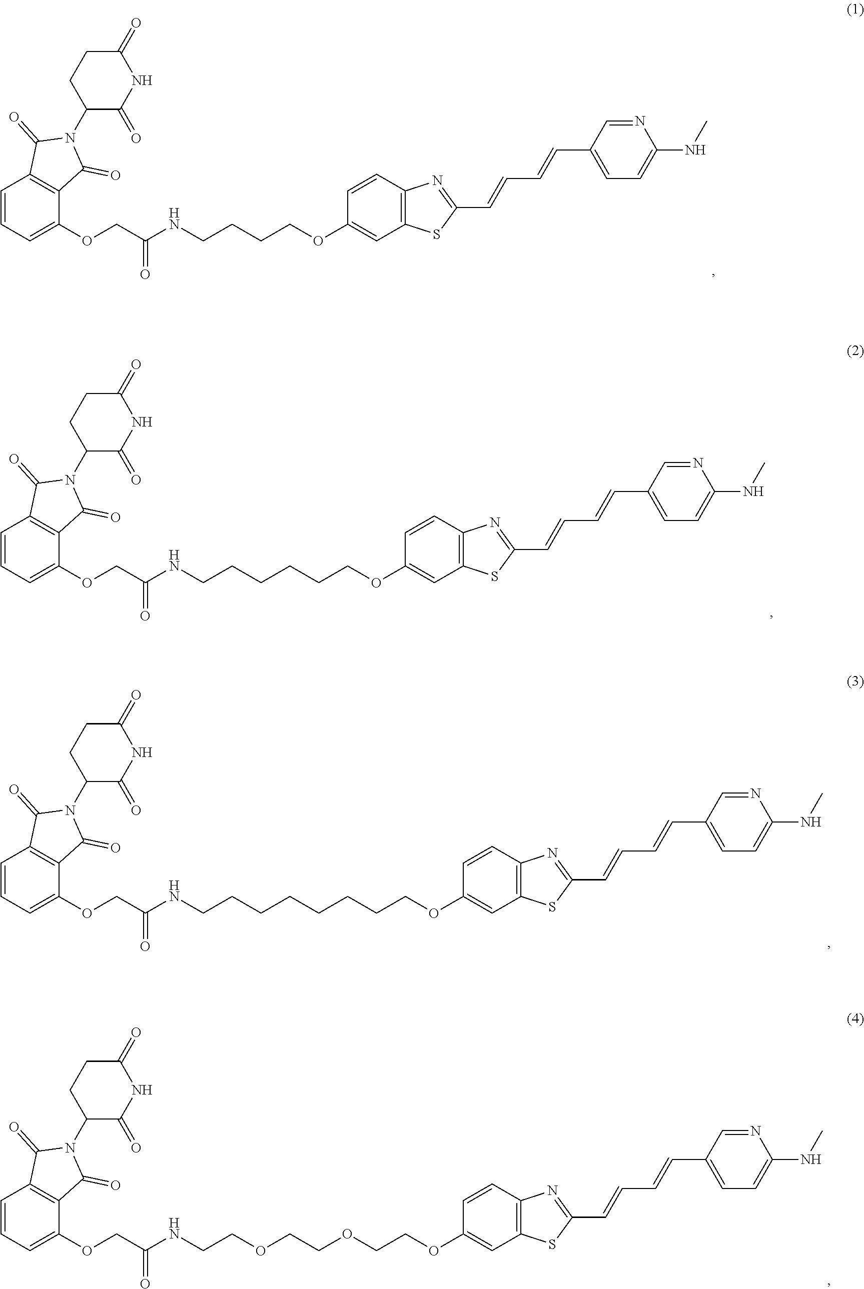 [SMILES: CNc1ccc(/C=C/C=C/c2nc3ccc(OCCCCNC(=O)COc4cccc5c4C(=O)N(C4CCC(=O)NC4=O)C5=O)cc3s2)cn1 ; CNc1ccc(/C=C/C=C/c2nc3ccc(OCCCCCCNC(=O)COc4cccc5c4C(=O)N(C4CCC(=O)NC4=O)C5=O)cc3s2)cn1 ; CNc1ccc(/C=C/C=C/c2nc3ccc(OCCCCCCCCNC(=O)COc4cccc5c4C(=O)N(C4CCC(=O)NC4=O)C5=O)cc3s2)cn1 ; CNc1ccc(/C=C/C=C/c2nc3ccc(OCCOCCOCCNC(=O)COc4cccc5c4C(=O)N(C4CCC(=O)NC4=O)C5=O)cc3s2)cn1]
or a pharmaceutically acceptable salt or stereoisomer thereof.

23. A pharmaceutical composition, comprising a therapeutically effective amount of the bispecific compound or pharmaceutically acceptable salt or stereoisomer thereof of claim 1, and a pharmaceutically acceptable carrier.

24. The pharmaceutical composition of claim 23, wherein the pharmaceutically acceptable carrier is a solid or a liquid.

25. (canceled)

26. The method of treating a disease or disorder that is characterized or mediated by aberrant activity of tau protein, comprising administering to a subject in need thereof a therapeutically effective amount of the bispecific compound or a pharmaceutically acceptable salt or stereoisomer thereof of claim 1.

27. The method of claim 26, wherein the disease or disorder is a neurodegenerative disease or disorder, a neuropsychiatric disease or disorder, a neurological disease or disorder, or a retinal disease or disorder.

28. The method of claim 27, wherein the neurodegenerative disease is Parkinson's disease, Prion disease, Huntington's disease, Alzheimer's disease, multiple system atrophy, Pick's disease, progressive supranuclear palsy (PSP), frontotemporal dementia (FTD), corticobasal degeneration (CBD), chronic traumatic encephalopathy, argyrophilic grains disease, tangle-dominant dementia, or primary age-related tauopathy (PART).

29. (canceled)

30. The method of claim 27, wherein the neuropsychiatric disease or disorder is autism, schizophrenia, bipolar disorder, an attention deficit disorder, a cognitive deficit disorder, palsy, or depression.

31. (canceled)

32. The method of claim 27, wherein the neurological disease or disorder is infantile tauopathy.

33. The method of claim 26, wherein the disorder is epilepsy or seizure.

34. (canceled)

35. The method of claim 27, wherein the retinal disease or disorder is glaucoma.
